SEQUENCE LISTING (30 sequences)

SEQ ID NO: 1 - protein, 252 aa, Escherichia coli
MNKFEDIRGVAFDLDGTLVDSAPGLAAAVDMALYALELPVAGEERVITWIGNGADVLMERALTWARQERATQRKTMGKPPVDDDIPAEEQVRILRKLFDRYYGEVAEEGTFLFPHVADTLGALQAKGLPLGLVTNKPTPFVAPLLEALDIAKYFSVVIGGDDVQNKKPHPDPLLLVAERMGIAPQQMLFVGDSRNDIQAAKAAGCPSVGLTYGYNYGEAIDLSQPDVIYQSINDLLPALGLPHSENQESKND

SEQ ID NO: 2 - protein, 411 aa, Mycobacterium avium
MNSPPKVSVLITVTGVDQPGVTATLFEVLSGHGVELLNVEQVVIRHRLTLGVLVCCPADVADGPALRHDVEAAIRKVGLDVSIERSDDVPIIREPSTHTIFVLGRPITAAAFGAVAREVAALGVNIDLIRGVSDYPVIGLELRVSVPPGADGALRTALNRVSSEEHVDVAVEDYTLERRAKRLIVFDVDSTLVQGEVIEMLAAKAGAEGQVAAITDAAMRGELDFAQSLQQRVATLAGLPATVIDEVAGQLELMPGARTTLRTLRRLGYACGVVSGGFRRIIEPLAEELMLDYVAANELEIVDGTLTGRVVGPIIDRAGKATALREFAQRAGVPMAQTVAVGDGANDIDMLAAAGLGIAFNAKPALREVADASLSHPYLDTVLFLLGVTRGEIEAADAIDGEVRRVEIPPE

SEQ ID NO: 3 - protein, 224 aa, Haemophilus sp.
MTQFKLIGFDLDGTLVNSLPDLALSINSALKDVNLPQASENLVMTWIGNGADVLSQRAVDWACKQAEKELTEDEFKYFKRQFGFYYGENLCNISRLYPNVKETLEALKAQGYILAVVTNKPTKHVQPILTAFGIDHLFSEMLGGQSLPEIKPHPAPFYYLCGKFGLYPKQILFVGDSQNDIFAAHSAGCAVVGLTYGYNYNIPIAQSKPDWIFDDFADILKITQ

SEQ ID NO: 4 - protein, 250 aa, Saccharomyces cerevisiae
MGLTTKPLSLKVNAALFDVDGTIIISQPAIAAFWRDFGKDKPYFDAEHVIQVSHGWRTFDAIAKFAPDFANEEYVNKLEAEIPVKYGEKSIEVPGAVKLCNALNALPKEKWAVATSGTRDMAQKWFEHLGIRRPKYFITANDVKQGKPHPEPYLKGRNGLGYPINEQDPSKSKVVVFEDAPAGIAAGKAAGCKIIGIATTFDLDFLKEKGCDIIVKNHESIRVGGYNAETDEVEFIFDDYLYAKDDLLKW

SEQ ID NO: 5 - protein, 321 aa, Homo sapiens
MAAAEAGGDDARCVRLSAERAQALLADVDTLLFDCDGVLWRGETAVPGAPEALRALRARGKRLGFITNNSSKTRAAYAEKLRRLGFGGPAGPGASLEVFGTAYCTALYLRQRLAGAPAPKAYVLGSPALAAELEAVGVASVGVGPEPLQGEGPGDWLHAPLEPDVRAVVVGFDPHFSYMKLTKALRYLQQPGCLLVGTNMDNRLPLENGRFIAGTGCLVRAVEMAAQRQADIIGKPSRFIFDCVSQEYGINPERTVMVGDRLDTDILLGATCGLKTILTLTGVSTLGDVKNNQESDCVSKKKMVPDFYVDSIADLLPALQG

SEQ ID NO: 6 - protein, 550 aa, Synechococcus elongatus
MKIKLLCISLAVLFCSSANAQKKQAKVQPSVFPQTVARPKLVVGMVIDQMRWDYLYRFYARYGNGGFKRLINEGFSAENTLIPYTPTLTACGHSSIYTGSVPAINGIIGNNWFDPQLGRDVYCVEDKSVKTVGSSSNEGLMSPKNLLVTTVTDELRMATNFRSKVISVSIKDRGAILPGGHTANGAYWYDDMTGSFISSTHYMQQLPTWVNDFNAQRLPNKYFEQDWNTLYPIETYTESTADAKPYERTFKGAKTSSFPHLFKQYANKNYSMMASMPQGNSFTLEFAKAAIPAEKLGQTGNTDFLAVSLSSTDYVGHQFGPNSIELEDTYLRLDKDLEDFFNYLDKTIGKGNYLLFLTADHGATHVPGFLRNKMPGGRLLLKVQTDLDSLIFNEFKVRCNFTIINNQVIFDTDAIKEAKADYAKIKQSTIDYLVKQDGVLNAVDIKNMGAVTIPQEIKNKIINGYNARRSGDVYIILDAGWYPTLTPGTGHAAWNPYDSHIPALFMGWGVKPGKTNKEYYMSDIAPTVSALLHIQQPSGSIGKVITDLLK

SEQ ID NO: 7 - protein, 321 aa, synthetic construct
MMTTTMMPIKITTKEQVQEFLDKYDTFLFDCDGVLWLGDHLLPSVKETLDLLKSLGKQLIFVTNNSTKSRAAYTKKFAKFGITGVTEDEIFGSSYASAVYVRDFLKLPPGKDKVWVFGESGIEEELKEMGYETLGGTDPRLDEPGVKFDADTSPFLVNGLDPDVGCVVAGLDTKINYHRLAITLQYLQKDNPSVPFVATNIDSTFPQKGKLLPGAGSIIESVAFASGRQPDAACGKPNQNMLNSIKAAKPGLNLDPSRCCMVGDRLNTDMKFGRDGGLGGTLLVLTGIETEERALALSDEHPAPKYYADKLGDLYELTHNN

SEQ ID NO: 8 - protein, 266 aa, Escherichia coli
MYQVVASDLDGTLLSPDHTLSPYAKETLKLLTARGINFVFATGRHHVDVGQIRDNLEIKSYMITSNGARVHDLDGNLIFAHNLDRDIASDLFGVVNDNPDIITNVYRDDEWFMNRHRPEEMRFFKEAVFQYALYEPGLLEPEGVSKVFFTCDSHEQLLPLEQAINARWGDRVNVSFSTLTCLEVMAGGVSKGHALEAVAKKLGYSLKDCIAFGDGMNDAEMLSMAGKGCIMGSAHQRLKDLHPELEVIGTNADDAVPHYLRKLYLS

SEQ ID NO: 9 - protein, 312 aa, Saccharomyces cerevisiae
MTAQQGVPIKITNKEIAQEFLDKYDTFLFDCDGVLWLGSQALPYTLEILNLLKQLGKQLIFVTNNSTKSRLAYTKKFASFGIDVKEEQIFTSGYASAVYIRDFLKLQPGKDKVWVFGESGIGEELKLMGYESLGGADSRLDTPFDAAKSPFLVNGLDKDVSCVIAGLDTKVNYHRLAVTLQYLQKDSVHFVGTNVDSTFPQKGYTFPGAGSMIESLAFSSNRRPSYCGKPNQNMLNSIISAFNLDRSKCCMVGDRLNTDMKFGVEGGLGGTLLVLSGIETEERALKISHDYPRPKFYIDKLGDIYTLTNNEL

SEQ ID NO: 10 - protein, 300 aa, Candida krusei
  source 1..300: note = Organism also commonly known as Pichia kudriavzevii
MTIKIATRGAAEKILDKYDTYLFDCDGVIWIGNELLPSVKETLELLQSKKKNLIFVSNNSTKARDQYIEKLAGLGIHGVELEQIINSCYSTAIYINEVLQLDKAKKIWVLGQEGITKEVEKFGYETVTFKDLEAKFQEEELTVDNIQSLIDPQVGCVIVGLDFQVTYLKIAITQQYLFDRKIPFIATNIDSTFPFGKTKLPGAGSIVEVASFCSGREPDAICGKPQSGMMDSILSHHSLNKETTIMVGDRLNTDMKFGSVNGIDTLLVLTGIEVEESVANGHSDVTYYADKLGDLYDLTK

SEQ ID NO: 11 - protein, 384 aa, Candida krusei
  source 1..384: note = Organism also commonly known as Pichia kudriavzevii
MTNSEIQRMSTPVEEVSQQLESKQDIGFVFDIDGVLLKGKKRIVEATEIITYLQKEKIPFILLTNGGGMTEKKRIDFINMTLQLETPIHPDQLIQAHTPMKTLIPHHKRVLICGGPKDDVREVAESYGFEDVIRPVDIIRANPTIWPYHMFTDEQIEQWGRDPNDTKLDVDGTGCKENLPIDSILCFNDSRALGAEIQIISDLLNSENGVLGTRRTVKSSKPSIPIVFSNNDFLWANEFVQPRFAMGVLKIATQTIYSKINGGEELEQLTLGKPEKVCYDYAHHILIDWRDVLLGKKKAGNVCLPQLNNEPLNSPFKKVYMVGDNPESDITGGNRYNWGTILVKTGVYQEGDFERNPEISKPSLGVFANVKEGVMHALKLNGLA

SEQ ID NO: 12 - protein, 362 aa, Candida krusei
  source 1..362: note = Organism also commonly known as Pichia kudriavzevii
MLSLQFKRLIHSIKKSPVGFAFDIDGVLMQSKNPILPYARDTLTLLQDHKIPFVLLTNGGGQTEASRVSFLNKSLSLKNPLRTSQIVLSHTPMRNLTGKYNRVLVVGGDDPQTREVAQSYGFQEVIRPIDIVREAPNIWPFIRYTREEIQALSKPVDLRQPIDAAFVFNDPRDMGTDTQIVIDALCSENGVLGTRRGIGSSKPSVPIIFSNMDLLWSTGYPIPRFGQGAFRLTIRELYKQLNNAELEDIVLGKPWPVSYTYAENVLNDEWKLLNGDRSGANEIPELGVKPKEQLVENVYMVGDNPQSDISGGNNYGWETILVETGVYKEGDFHKYPTLAKPTFGIFPNVWDGVTSALKRHQV

SEQ ID NO: 13 - protein, 352 aa, Saccharomyces cerevisiae
MIGKRFFQTTSKKIAFAFDIDGVLFRGKKPIAGASDALKLLNRNKIPYILLTNGGGFSERARTEFISSKLDVDVSPLQIIQSHTPYKSLVNKYSRILAVGTPSVRGVAEGYGFQDVVHQTDIVRYNRDIAPFSGLSDEQVMEYSRDIPDLTTKKFDAVLVFNDPHDWAADIQIISDAINSENGMLNTLRNEKSGKPSIPIYFSNQDLLWANPYKLNRFGQGAFRLLVRRLYLELNGEPLQDYTLGKPTKLTYDFAHHVLIDWEKRLSGKIGQSVKQKLPLLGTKPSTSPFHAVFMVGDNPASDIIGAQNYGWNSCLVKTGVYNEGDDLKECKPTLIVNDVFDAVTKTLEKYA

SEQ ID NO: 14 - protein, 249 aa, Candida krusei
MVHKLILVRHGQSEWNEKNLFTGWVDVKLSEKGRQEAVRAGELLKEADIKPDVLYTSLLTRAIQTANIALENADRLHIPVVRSWRLNERHYGALQGKDKAQTLETYGPEKFQQWRRSFDIPPPPIEDHSEFSQFGEERYSNVDPNVLPKTESLALVIDRLLPFWQDVIAKDLLANKTVIITAHGNSLRGLVKHLDNISDDDIAGLNIPTAIPLVYELDDNLKPIKPAYYLDPEAAAAGAAAVAAQGQKK

SEQ ID NO: 15 - protein, 469 aa, Saccharomyces cerevisiae
MTSIDINNLQNTFQQAMNMSGSPGAVCTSPTQSFMNTVPQRLNAVKHPKILKPFSTGDMKILLLENVNQTAITIFEEQGYQVEFYKSSLPEEELIEKIKDVHAIGIRSKTRLTSNVLQHAKNLVCIGCFCIGTNQVDLDYATSRGIAVFNSPFSNSRSVAELVIAEIISLARQLGDRSIELHTGTWNKVAARCWEVRGKTLGIIGYGHIGSQLSVLAEAMGLHVLYYDIVTIMALGTARQVSTLDELLNKSDFVTLHVPATPETEKMLSAPQFAAMKDGAYVINASRGTVVDIPSLIQAVKANKIAGAALDVYPHEPAKNGEGSFNDELNSWTSELVSLPNIILTPHIGGSTEEAQSSIGIEVATALSKYINEGNSVGSVNFPEVALKSLSYDQENTVRVLYIHQNVPGVLKTVNDILSNHNIEKQFSDSNGEIAYLMADISSVDQSDIKDIYEQLNQTSAKISIRLLY

SEQ ID NO: 16 - protein, 381 aa, Escherichia coli
MKIVIAPDSFKESLSAEKCCQAIKAGFSTLFPDANYICLPIADGGEGTVDAMVAATGGNIVTLEVCGPMGEKVNAFYGLTGDGKTAVIEMAAASGLMLVAPEKRNPLLASSFGTGELIRHALDNDIRHIILGIGGSATVDGGMGMAQALGVRFLDADGQALAANGGNLARVASIEMDECDPRLANCHIEVACDVDNPLVGARGAAAVFGPQKGATPEMVEELEQGLQNYARVLQQQTEINVCQMAGGGAAGGMGIAAAVFLNADIKPGIEIVLNAVNLAQAVQGAALVITGEGRIDSQTAGGKAPLGVASVAKQFNVPVIGIAGVLGDGVEVVHQYGIDAVFSILPRLAPLAEVLASGETNLFNSARNIACAIKIGQGIKN

SEQ ID NO: 17 - protein, 486 aa, synthetic construct
MSSPQDINNLQNDLLARARSFQQALNLSGSPGNAVSTSPPTSFGGSFMNQLPRRLSRTAASKPAKALKPFSTGDIKILLLENVNQTARDIFKEQGYQVEFLKSSLPEDELIEKIRDVHAIGIRSKTKLTAKVLKHAKNLIVIGCFCIGTNQVDLEYAAKHGIAVFNSPFSNSRSVAELVIAEIISLARQLGDRSIEMHTGTWNKVSAKCWEIRGKTLGIIGYGHIGSQLSVLAEAMGMNVIYYDVVTIMALGTAKQVPTLDELLNKADFVTLHVPETPETKNMISAPQFAAMKDGAYLINASRGTVVDIPALIQAMKSGKIAGAALDVYPNEPAKNGDGYFNDDLNSWASELRSLKNVILTPHIGGSTEEAQSAIGVEVATALVKYINEGNSLGAVNFPEVSLRSLTLDQPNHVRVLYIHQNVPGVLRTVNEILSNHNIEKQFSDSRGDIAYLMADISDVDQSDIKDLYEQLEQTSSKISTRLLYL

SEQ ID NO: 18 - protein, 381 aa, Escherichia coli
MKIVIAPDSYKESLSASEVAQAIEKGFREIFPDAQYVSVPVADGGEGTVEAMIAATQGAERHAWVTGPLGEKVNASWGISGDGKTAFIEMAAASGLELVPAEKRDPLVTTSRGTGELILQALESGATNIIIGIGGSATNDGGAGMVQALGAKLCDANGNEIGFGGGSLNTLNDIDISGLDPRLKDCVIRVACDVTNPLVGDNGASRIFGPQKGASEAMIVELDNNLSHYAEVIKKALHVDVKDVPGAGAAGGMGAALMAFLGAELKSGIEIVTTALNLEEHIHDCTLVITGEGRIDSQSIHGKVPIGVANVAKKYHKPVIGIAGSLTDDVGVVHQHGIDAVFSVLTSIGTLDEAFRGAYDNICRASRNIAATLAIGMRNAG

SEQ ID NO: 19 - protein, 388 aa, Candida krusei
  source 1..388: note = Organism also commonly known as Pichia kudriavzevii
MVSPAERLSTIASTIKPNRKDSTSLQPEDYPEHPFKVTVVGSGNWGCTIAKVIAENTVERPRQFQRDVNMWVYEELIEGEKLTEIINTKHENVKYLPGIKLPVNVVAVPDIVEACAGSDLIVFNIPHQFLPRILSQLKGKVNPKARAISCLKGLDVNPNGCKLLSTVITEELGIYCGALSGANLAPEVAQCKWSETTVAYTIPDDFRGKGKDIDHQILKSLFHRPYFHVRVISDVAGISIAGALKNVVAMAAGFVEGLGWGDNAKAAVMRIGLVETIQFAKTFFDGCHAATFTHESAGVADLITTCAGGRNVRVGRYMAQHSVSATEAEEKLLNGQSCQGIHTTREVYEFLSNMGRTDEFPLFTTTYRIIYENFPIEKLPECLEPVED

SEQ ID NO: 20 - protein, 563 aa, Candida krusei
  source 1..563: note = Organism also commonly known as Pichia kudriavzevii
MIPRLNPLLNISHLRGGPKFIGKAIKPSQFEFRKNNFRFNSTSTKTGSARTIKSGFLSWSFRAATFTGIAGWLYLTYLVYKETNPGSQSPQTEFSEIGNKKKNIVILGSGWGAVSVLKTLDTTKYNVTIVSPRNYFLFTPLLPSVPSGTIDIKSICDSIRTIARQTPGEVTYLEAAATDIDPVKKTIKLEHKSQRFLIGDAFTSEGDVIENELSYDYLVYAVGATVNTFGIPGIPEYASYLKEANDATAVRQKLFNQIEASRLLPKDSEDRKRLLSFVVCGGGPTGVELAAEIKDYIDQDLCKFIPGIEKEMQVTLIEAQHNVLSMFHPKLIEYTKEVFKQQNLHLQVDTMVKKVDDKNVYATYRHPDGKTEDMVIPYGTLVWAGGNAQRKLTRDLSSKIIEQKTARRGLLVDEYLKLDGDDSIYAIGDCTFTPNPPTAQVAHQQGEYLGEHFNKLAKIDELNYLITNSTDDSTKYSKRLERAEKAIKPFEYDHQGALAYVGSERAVADLHWGSWSTVALGGTMTFFFWRTAYVSMLLSIRNKILVVTDWVKVAIFGRDCSQE

SEQ ID NO: 21 - protein, 446 aa, Lactococcus lactis
MKIVVIGTNHAGIATANTLIDRYPGHEIVMIDRNSNMSYLGCGTAIWVGRQIEKPDELFYAKAEDFEKKGVKILTETEVSEIDFTNKMIYAKSKTGEKITESYDKLVLATGSRPIIPNLPGKDLKGIHFLKLFQEGQAIDEEFAKNDVKRIAVIGAGYIGTEIAEAAKRRGKEVLLFDAESTSLASYYDEEFAKGMDENLAQHGIELHFGELAQEFKANEKGHVSQIVTNKSTYDVDLVINCIGFTANSALAGEHLETFKNGAIKVDKHQQSSDPDVSAVGDVATIYSNALQDFTYIALASNAVRSGIVAGHNIGGKSIESVGVQGSNGISIFGYNMTSTGLSVKAAKKIGLEVSFSDFEDKQKAWFLHENNDSVKIRIVYETKNRRIIGAQLASKSEIIAGNINMFSLAIQEKKTIDELALLDLFFLPHFNSPYNYMTVAALNAK

SEQ ID NO: 22 - protein, 1511 aa, Saccharomyces cerevisiae
MSSTDEHIEKDISSRSNHDDDYANSVQSYAASEGQVDNEDLAATSQLSRHLSNILSNEEGIERLESMARVISHKTKKEMDSFEINDLDFDLRSLLHYLRSRQLEQGIEPGDSGIAFKNLTAVGVDASAAYGPSVEEMFRNIASIPAHLISKFTKKSDVPLRNIIQNCTGVVESGEMLFVVGRPGAGCSTFLKCLSGETSELVDVQGEFSYDGLDQSEMMSKYKGYVIYCPELDFHFPKITVKETIDFALKCKTPRVRIDKMTRKQYVDNIRDMWCTVFGLRHTYATKVGNDFVRGVSGGERKRVSLVEAQAMNASIYSWDNATRGLDASTALEFAQAIRTATNMVNNSAIVAIYQAGENIYELFDKTTVLYNGRQIYFGPADKAVGYFQRMGWVKPNRMTSAEFLTSVTVDFENRTLDIKPGYEDKVPKSSSEFEEYWLNSEDYQELLRTYDDYQSRHPVNETRDRLDVAKKQRLQQGQRENSQYVVNYWTQVYYCMIRGFQRVKGDSTYTKVYLSSFLIKALIIGSMFHKIDDKSQSTTAGAYSRGGMLFYVLLFASVTSLAEIGNSFSSRPVIVKHKSYSMYHLSAESLQEIITEFPTKFVAIVILCLITYWIPFMKYEAGAFFQYILYLLTVQQCTSFIFKFVATMSKSGVDAHAVGGLWVLMLCVYAGFVLPIGEMHHWIRWLHFINPLTYAFESLVSTEFHHREMLCSALVPSGPGYEGISIANQVCDAAGAVKGNLYVSGDSYILHQYHFAYKHAWRNWGVNIVWTFGYIVFNVILSEYLKPVEGGGDLLLYKRGHMPELGTENADARTASREEMMEALNGPNVDLEKVIAEKDVFTWNHLDYTIPYDGATRKLLSDVFGYVKPGKMTALMGESGAGKTTLLNVLAQRINMGVITGDMLVNAKPLPASFNRSCGYVAQADNHMAELSVRESLRFAAELRQQSSVPLEEKYEYVEKIITLLGMQNYAEALVGKTGRGLNVEQRKKLSIGVELVAKPSLLLFLDEPTSGLDSQSAWSIVQFMRALADSGQSILCTIHQPSATLFEQFDRLLLLKKGGKMVYFGDIGPNSETLLKYFERQSGMKCGVSENPAEYILNCIGAGATASVNSDWHDLWLASPECAAARAEVEELHRTLPGRAVNDDPELATRFAASYMTQIKCVLRRTALQFWRSPVYIRAKFFECVACALFVGLSYVGVNHSVGGAIEAFSSIFMLLLIALAMINQLHVFAYDSRELYEVREAASNTFHWSVLLLCHAAVENFWSTLCQFMCFICYYWPAQFSGRASHAGFFFFFYVLIFPLYFVTYGLWILYMSPDVPSASMINSNLFAAMLLFCGILQPREKMPAFWRRLMYNVSPFTYVVQALVTPLVHNKKVVCNPHEYNIMDPPSGKTCGEFLSTYMDNNTGYLVNPTATENCQYCPYTVQDQVVAKYNVKWDHRWRNFGFMWAYICFNIAAMLICYYVVRVKVWSLKSVLNFKKWFNGPRKERHEKDTNIFQTVPGDENKITKK

SEQ ID NO: 23 - protein, 944 aa, Saccharomyces cerevisiae
MDTQIAITGVAVGKEINNDNSKTDQKVSLPKADVPCIDKATQTIIEGCSKDDPRLSYPTKLETTEKGKTKRNSFACVCCHSLKQKCEPSDVNDIYRKPCRRCLKHKKLCKFDLSKRTRKRKPRSRSPTPFESPMVNVSTKSKGPTDSEESSLKDGTSYLASFPSDPNAKQFPNSRTVLPGLQQSLSDLWSTLSQPPSYGAREAETTSTGEITTNNHTKSNGSVPTNPAVLASNDEHTNISDAPVIYSTYNSPVPISSAPTSINSEALFKHRPKIVGDEETQNVKVKRQKKSYSRHMTRSFRKQLQSLIISQKGKIRDISMKLDTWSKQWNDLVEKSMFLPTIADPVSVGIISHEEATLRLHLYKTEISYLSKLPFIKVEENVSVDELRKKKPILFSVIMSCVSIVLTPKQTTRGTIMKLDSFVLNLITNQIFKANNKSIEIIESLSTLCLWYNFFEWSSKTRYHIFNYICCCLTRDLGPTYVNRSFGMFSDEDPKRFKSPLELYSNGASLTLLVYISALNISIFLRQSIQARWSHVTEKACEDLVKETKKSRHYDNDKLLLDSADDPILVQFAKMNHVLENIHTHLHERDLNDDEFDDPIFTKKYLNKLMEKYHKQLQEIFTKLDRNRPRVIAFYYSVEAYLYQYKLAVFIGEMSHTINEKVELPREIMDDFVKCYHCCKSALEEFSKLEPILITSLPLFHTSRIIYTVGMLLLKLRYSVVAIPSFHDLMPLTDDAIALVIGVNNLLEKTSELYPFNNSLYKFRYVIALFCQTYANKVIDVADRYNAEREKLKEKQVIDEVSNGHDGTKPINAYVTESQKMPTEEDPIIDNNTNQNITAVPDEMLPVYSRVRDDTAAMNLNINSTSYMNESPHEHRESMTGTTLLPPPFISNDVTNSADSTNIKPSPSSSVDNLNDYLTDINSLAWGVNSLNDEFWTDLFMNDI

SEQ ID NO: 24 - protein, 1517 aa, Kluyveromyces sp.
  source 1..1517: note = Organism commonly known as Kluyveromyces marxianus or Candida kefyr
MSNSSSSEGKTNEDGRNSVHSSDSFAQSVASFHLDDNESQNVTAQLSQQITNVLSNSNGAERIESLARVISTKTKKQMESFEVNQLDFDLKALLNYLRSSQLEQGIEPGDSGIAFHDLTAVGIDASAAFGPSVEEMVRSWIHFPVRLWKKICRQKSETPLRNIIQHCTGVVESGEMLFVVGRPGAGCSTLLKCLSGETGELVEVTGDISYDGLSQEEMMQKFKGYVIYCPELDFHFPKITVKETIDFALKCKTPRSRIDHLTRAQYVDNMRDLWCTVFGLTHTYATNVGNDVVRGVSGGERKRVSLVEALAMNASIYSWDNATRGLDASTALEFAQAIRTATNMMNNSAIVAIYQAGENIYQLFDKTTVLYNGKQVYFGPADEAVGYFERMGYIKPNRMTSAEFLTSATVDFENRTLEVREGYEEKIPKSSTEMEAYWHNSPEYAKATELFNEYCQSHPEEETRQRLETAKKQRLQKGQREKSQFVVTFWAQVWYCMIRGFQRVKGDSTYTKVYLSSFLTKGLIVGSMFHKIDPKSQSTTEGAYSRGGLLFYVLLFAALTSLAEISNSFQNRAIIVKQKTYSMYHTSAESLQEIFTEIPTKFVAILTLSLVSYWIPVLKYDAGSFFQYLLYLFTTQQCTSFIFKLVATLTKDGGTAHAIGGLWVLMLTVYAGFVLPIGNMHHWIRWFHYLNPLTYAYESLMSTEFHGRKMLCSRLLPSGPGYENVSIAHKICDAAGAVAGQLYVSGDAYVLKKYHFRYKHAWRDWGINIVWTFGYIVMNVVMSEYLKPLEGGGDLLLYKRGHMPELGSESVDSKVASREEMMESLNGPGVDLEKVIASKDVFTWNHLNYTIPYDGATRQLLSDVFGYVKPGKMTALMGESGAGKTTLLNVLAQRINVGVITGDMLVNAKPLPPSFNRSCGYVAQADNHMGELSVRESLRFAAELRQPKSVPLQEKYDYVEKIISLLGMEKYAEAIIGKTGRGLNVEQRKKLSIGVELVAKPSLLLFLDEPTSGLDSQSAWSIVQFMRALADSGQSILCTIHQPSATLFEQFDRLLLLKKGGKMVYFGDIGENSSTLLNYFERQSGVKCGKSENPAEYMLNCIGAGATASADADWHDLWLQSPECAAAREEVEELHRTLASRPVTDDKELAGRYAASYLTQMKCVFRRTNIQFWRSPVYIRAKFLECVLCALFVGLSYVGVDHSIAGASQSFSSIFMMLLIALAMVNQLHVFALDSRELYEVREAASNTFHWSVLLLNHTFVEIIWSTLCEFICWICYYWPAQYSGRASHAGYFFLIYVIMFPAYFVSYGCWVFYMSPDVPSASMINSNLFAGMLLFCGILQPKDKMPGFWKRFMYNVSPFTYVVQSLVTPLVQGKKVRCTKNEFAVVNPPEGQTCSQYFARFIKDNTGYLKNPNDTESCHYCPYSYQQEVVEQYNVRWVYRWRNFGFLWAYIGFNFFAMLACYWVLRVKNYSITSIFGVFKIGNWKKAIHHDSRHEKDHTIFQEKPGDAANVQKTKA

SEQ ID NO: 25 - protein, 261 aa, synthetic construct
MSSLKPKIKAISMMPKLVLVRHGQSEWNEKNLFTGWVDVKLSAVGEKEAARAGELLKEANIKPDVLYTSKLSRAIQTANIALEKADRLWIPVKRSWRLNERHYGALQGKDKAETLEQYGEEKFQTWRRSFDVPPPPIEDDSEFSQKGDERYADVDPAVLPKTESLALVIDRLLPYWQDVIAKDLLAGKTVLIAAHGNSLRALVKHLDNISDADIAGLNIPTGIPLVYELDENLKPTKPAYYLDPEAAAAGAAAVAAQGKKK

SEQ ID NO: 26 - protein, 441 aa, synthetic construct
MAISKIHARSVYDSRGNPTVEVDLTTEKGLFRAIVPSGASTGIHEALELRDGDKSKWLGKGVLKAVANVNDVIAPALIKANIDVKDQSKVDEFLIKLDGTPNKSKLGANAILGVSLAAAKAAAAEKGVPLYAHIADLAGTKTKPYVLPVPFQNVLNGGSHAGGALAFQEFMIVPTGAPSFSEALRIGSEVYHNLKSLAKKKYGQSAGNVGDEGGVAPDIQTAEEALDLIVDAIEAAGYTGKVKIALDVASSEFYKDDAGKYDLDFKNPESDPSKWLTGEQLADLYHSLAKKYPIVSIEDPFAEDDWEAWSHFYKTAGSDIQIVGDDLTVTNPIRIKTAIEKKAANALLLKVNQIGTLTESIQAAKDSYAAGWGVMVSHRSGETEDTFIADLVVGLRTGQIKTGAPARSERLAKLNQILRIEEELGDKAIYAGENFHTAVNL

SEQ ID NO: 27 - protein, 438 aa, Schizosaccharomyces pombe Tf1 virus
MGELKEILKQRYHELLDWNVKAPHVPLSQRLKHFTWSWFACTMATGGVGLIIGSFPFRFYGLNTIGKIVYILQIFLFSLFGSCMLFRFIKYPSTIKDSWNHHLEKLFIATCLLSISTFIDMLAIYAYPDTGEWMVWVIRILYYIYVAVSFIYCVMAFFTIFNNHVYTIETASPAWILPIFPPMICGVIAGAVNSTQPAHQLKNMVIFGILFQGLGFWVYLLLFAVNVLRFFTVGLAKPQDRPGMFMFVGPPAFSGLALINIARGAMGSRPYIFVGANSSEYLGFVSTFMAIFIWGLAAWCYCLAMVSFLAGFFTRAPLKFACGWFAFIFPNVGFVNCTIEIGKMIDSKAFQMFGHIIGVILCIQWILLMYLMVRAFLVNDLCYPGKDEDAHPPPKPNTGVLNPTFPPEKAPASLEKVDTHVTSTGGESDPPSSEHESV

SEQ ID NO: 28 - protein, 437 aa, Saccharomyces cerevisiae
MAVSKVYARSVYDSRGNPTVEVELTTEKGVFRSIVPSGASTGVHEALEMRDGDKSKWMGKGVLHAVKNVNDVIAPAFVKANIDVKDQKAVDDFLISLDGTANKSKLGANAILGVSLAASRAAAAEKNVPLYKHLADLSKSKTSPYVLPVPFLNVLNGGSHAGGALALQEFMIAPTGAKTFAEALRIGSEVYHNLKSLTKKRYGASAGNVGDEGGVAPNIQTAEEALDLIVDAIKAAGHDGKIKIGLDCASSEFFKDGKYDLDFKNPNSDKSKWLTGPQLADLYHSLMKRYPIVSIEDPFAEDDWEAWSHFFKTAGIQIVADDLTVTNPKRIATAIEKKAADALLLKVNQIGTLSESIKAAQDSFAAGWGVMVSHRSGETEDTFIADLVVGLRTGQIKTGAPARSERLAKLNQLLRIEEELGDNAVFAGENFHHGDKL

SEQ ID NO: 29 - protein, 437 aa, Saccharomyces cerevisiae
MAVSKVYARSVYDSRGNPTVEVELTTEKGVFRSIVPSGASTGVHEALEMRDEDKSKWMGKGVMNAVNNVNNVIAAAFVKANLDVKDQKAVDDFLLSLDGTANKSKLGANAILGVSMAAARAAAAEKNVPLYQHLADLSKSKTSPYVLPVPFLNVLNGGSHAGGALALQEFMIAPTGAKTFAEAMRIGSEVYHNLKSLTKKRYGASAGNVGDEGGVAPNIQTAEEALDLIVDAIKAAGHDGKVKIGLDCASSEFFKDGKYDLDFKNPESDKSKWLTGVELADMYHSLMKRYPIVSIEDPFAEDDWEAWSHFFKTAGIQIVADDLTVTNPARIATAIEKKAADALLLKVNQIGTLSESIKAAQDSFAANWGVMVSHRSGETEDTFIADLVVGLRTGQIKTGAPARSERLAKLNQLLRIEEELGDKAVYAGENFHHGDKL

SEQ ID NO: 30 - protein, 479 aa, Escherichia coli
MSVPVQHPMYIDGQFVTWRGDAWIDVVNPATEAVISRIPDGQAEDARKAIDAAERAQPEWEALPAIERASWLRKISAGIRERASEISALIVEEGGKIQQLAEVEVAFTADYIDYMAEWARRYEGEIIQSDRPGENILLFKRALGVTTGILPWNFPFFLIARKMAPALLTGNTIVIKPSEFTPNNAIAFAKIVDEIGLPRGVFNLVLGRGETVGQELAGNPKVAMVSMTGSVSAGEKIMATAAKNITKVCLELGGKAPAIVMDDADLELAVKAIVDSRVINSGQVCNCAERVYVQKGIYDQFVNRLGEAMQAVQFGNPAERNDIAMGPLINAAALERVEQKVARAVEEGARVAFGGKAVEGKGYYYPPTLLLDVRQEMSIMHEETFGPVLPVVAFDTLEDAISMANDSDYGLTSSIYTQNLNVAMKAIKGLKFGETYINRENFEAMQGFHAGWRKSGIGGADGKHGLHEYLQTQVVYLQS

(19) United States
(12) Patent Application Publication
(10) Pub. No.: US 2025/0333686 A1
(43) Pub. Date: Oct. 30, 2025

(54) RECOMBINANT HOST CELLS AND METHODS FOR THE PRODUCTION OF GLYCERIC ACID AND DOWNSTREAM PRODUCTS

(71) Applicant: Lygos, Inc., Hayward, CA (US)

(72) Inventors: Jeffrey A. Dietrich, Hayward, CA (US); Mario Ouellet, Hayward, CA (US); Johan van Walsem, Hayward, CA (US)

(73) Assignee: Lygos, Inc., Hayward, CA (US)

(21) Appl. No.: 18/961,371

(22) Filed: Nov. 26, 2024

Related U.S. Application Data
(63) Continuation of application No. 16/969,866, filed on Aug. 13, 2020, now Pat. No. 12,188,008.
(60) Provisional application No. 62/632,237, filed on Feb. 19, 2018.

(51) Int. Cl. C12N 1/16; C12N 1/20; C12N 9/02; C12N 9/12; C12N 9/16; C12N 9/88; C12N 9/90; C12P 7/42; C12R 1/72; C12R 1/84; C12R 1/865

(52) U.S. Cl. CPC C12N 1/16; C12N 1/20; C12N 9/0036; C12N 9/1205; C12N 9/16; C12N 9/88; C12N 9/90; C12P 7/42; C12N 2523/00; C12R 2001/72; C12R 2001/84; C12R 2001/865; C12Y 101/01008; C12Y 106/03001; C12Y 106/99003; C12Y 207/01031; C12Y 207/01165; C12Y 301/0302; C12Y 301/03038; C12Y 402/01011; C12Y 504/02001

(57) ABSTRACT

Methods and materials related to producing glyceric acid and downstream products are disclosed. Specifically, isolated nucleic acids. polypeptides, host cells, methods and materials for producing glycolic acid by direct fermentation from sugars are disclosed.

### CROSS-REFERENCE TO RELATED APPLICATIONS

This application is a continuation application of prior U.S. patent application Ser. No. 16/969,866, filed Aug. 13, 2020, now U.S. Pat. No. 12,188,008, issued Jan. 7, 2025, which is a National Stage of International Application No. PCT/US2019/018422, filed Feb. 18, 2019, and claims the benefit of priority under 35 U.S.C. 119(e) and Article 2 of the Paris Convention for the Protection of Industrial Property (1883) to U.S. provisional application Ser. No. 62/632,237, filed Feb. 19, 2018, the entire contents of which are incorporated herein in their entirety by this reference.

### REFERENCE TO SEQUENCE LISTING

This application contains a Sequence Listing submitted via EFS-web in computer readable form, and is hereby incorporated by reference in its entirety. The xml file, created on Jan. 31, 2025, is named LYGOS_0014_01_WO_ST26-Amended.xml and is 41.3 KB in size.

### BACKGROUND

The long-term economic and environmental concerns associated with the petrochemical industry have provided the impetus for increased research, development, and commercialization of processes for conversion of carbon feedstocks into chemicals that can replace those petroleum feedstocks. One approach is the development of biorefining processes to convert renewable feedstocks into products that can replace petroleum-derived chemicals. Two common goals in improving a biorefining process include achieving a lower cost of production and reducing detrimental effects on the environment. The present disclosure provides a safer and cheaper alternative to incumbent production methods that comprise hazardous petrochemicals and extreme process conditions.

Glyceric acid is an important precursor molecule that can be converted to high-value, biodegradable polymers which are in high demand in the food packaging, medical device and personal care industries. As glyceric acid has three functional groups (i.e., primary alcohol, secondary alcohol, and carboxylic acid) it is a valuable chemical building block for a range of applications, including polymers, superabsorbent polymers, and solvents.

Currently, the synthesis of glyceric acid and its derivatives requires a petroleum feedstock, extreme run temperatures and/or pressures, and hazardous materials that pose health and safety risks. Glyceric acid may be prepared by catalytic oxidation of glycerol with run temperatures as high as 350° C., and precious metal catalysts that comprise palladium and platinum. Likewise, glycolic acid may be prepared by hydrative carbonylation of formaldehyde with carbon monoxide and sulfuric acid, wherein run conditions range from 210° C. to 240° C. and ˜900 atm, or saponification of chloroacetic acid with alkali metal hydroxide, wherein run temperatures range from 100° C. to 160° C. Both formaldehyde and chloroacetic acid are recognized as toxic air contaminants by the U.S. Environmental Protection Agency and the California Air Toxics Program and commercial production of acrylic acid via oxidation of propylene requires run temperatures in the range of 250° C. to 450° C. Thus, there is a need for new low-cost, energy efficient, high yielding manufacturing methods for the synthesis of glyceric acid and its derivatives.

### SUMMARY

The present disclosure provides recombinant host cells and methods of their production and use to produce glyceric acid and downstream products and derivatives. These recombinant host cells utilize microbial fermentation from renewable feedstocks (for example, glucose) to produce glyceric acid. This glyceric acid production utilizes an efficient carbon conversion route; in cases where glucose is used as the raw material, the stoichiometric theoretical yield is 2-mols of glyceric acid for every mol of glucose, equating to one of the highest yielding products from glucose at 100% carbon conversion.

The microbial fermentation process disclosed herein for the production of glyceric acid is run at both ambient atmospheric pressure and temperature, reducing the cost and environmental impact of manufacturing relative to the incumbent petrochemical processes. No organism in nature, including yeast, is known to produce glyceric acid from glucose in more than trace amounts. The materials and methods described herein comprise a renewable and cheaper starting material and an environmentally-benign biosynthetic process. The present disclosure provides a safer and cheaper alternative to incumbent methods that comprise hazardous petrochemicals and extreme process conditions. The materials and methods described herein enable higher fermentation yields and productivities in the production of glyceric acid and its downstream products.

Thus, in various embodiments, the present disclosure provides recombinant host cells capable of producing glyceric acid comprising one or more heterologous nucleic acids that encode the glyceric acid biosynthetic pathway, wherein the pathway enzymes comprise a 3-phosphoglycerate phosphatase and/or a 2-phosphoglycerate phosphatase.

In other embodiments, this disclosure provides recombinant host cells that further comprise one or more heterologous nucleic acids encoding one or more ancillary proteins that function in redox cofactor recycling, redox cofactor biogenesis, or organic acid transport. In some embodiments, the one or more ancillary proteins comprise mitochondrial external NADH dehydrogenase, water-forming NADH oxidase, glyceric acid transporter, or combination thereof. In other embodiments, this disclosure provides recombinant host cells that further comprise a genetic disruption of one or more genes wherein the one or more genes encodes phosphoglycerate mutase, phosphoglycerate dehydrogenase, enolase, glycerate 3-kinase, glycerate 2-kinase, or glycerol-3-phosphate dehydrogenase, or combination thereof. In other embodiments, this disclosure provides recombinant host cells that further comprise one or more heterologous nucleic acids that encode a glycolic acid biosynthetic pathway, wherein the pathway enzymes comprise a hydroxypyruvate reductase, a hydroxypyruvate decarboxylase, and a glycoaldehyde dehydrogenase.

In other embodiments, this disclosure provides the invention provides a method for the production of glyceric acid that comprises culturing the recombinant host cells of the invention for a sufficient period of time to produce glyceric acid. In other embodiments, this disclosure provides a method for the production of glycolic acid that comprises culturing the recombinant host cells of the invention for a sufficient period of time to produce glycolic acid. In other embodiments, this disclosure provides a process to produce glycerate esters comprising the steps of recovering glyceric acid or glycerate salt from fermentation broth, forming the glycerate ester by esterification with supercritical CO₂, and purifying said glycerate ester. In an eighth aspect, the invention provides a deoxydehydration process to produce acrylate esters comprising the steps of converting a glycerate ester to an acrylate ester, and purifying said acrylate ester. In various embodiments, the deoxydehydration alcohol is methanol, ethanol, isopropyl alcohol, butanol, 3-pentanol, or isobutanol.

### DETAILED DESCRIPTION

The present disclosure provides recombinant host cells, materials and methods for the biological production, purification and synthesis of glyceric acid and downstream products.

While the present disclosure describes aspects and specific embodiments, those skilled in the art will recognize that various changes may be made and equivalents may be substituted without departing from this disclosure. The present disclosure is not limited to particular nucleic acids, expression vectors, enzymes, biosynthetic pathways, host microorganisms, or processes, as such may vary. The terminology used herein is for the purposes of describing particular aspects and embodiments only, and is not to be construed as limiting. In addition, many modifications may be made to adapt a particular situation, material, composition of matter, process, process steps or process flows, in accordance with this disclosure. All such modifications are within the scope of the claims appended hereto.

### Section 1: Definitions

In this specification and in the claims that follow, reference will be made to a number of terms that shall be defined to have the following meanings.

The term “accession number” and similar terms such as “protein accession number”, “UniProt ID”, “gene ID” and “gene accession number” refer to designations given to specific proteins or genes. These identifiers described a gene or protein sequence in publicly accessible databases such as the National Center for Biotechnology Information (NCBI).

The term “heterologous” as used herein refers to a material that is non-native to a cell. For example, a nucleic acid is heterologous to a cell, and so is a “heterologous nucleic acid” with respect to that cell, if at least one of the following is true: 1) the nucleic acid is not naturally found in that cell (that is, it is an “exogenous” nucleic acid); 2) the nucleic acid is naturally found in a given host cell (that is, “endogenous to”), but the nucleic acid or the RNA or protein resulting from transcription and translation of this nucleic acid is produced or present in the host cell in an unnatural (for example, greater or lesser than naturally present) amount; 3) the nucleic acid comprises a nucleotide sequence that encodes a protein endogenous to a host cell but differs in sequence from the endogenous nucleotide sequence that encodes that same protein (having the same or substantially the same amino acid sequence), typically resulting in the protein being produced in a greater amount in the cell, or in the case of an enzyme, producing a mutant version possessing altered (for example, higher or lower or different) activity; and/or 4) the nucleic acid comprises two or more nucleotide sequences that are not found in the same relationship to each other in the cell. As another example, a protein is heterologous to a host cell if it is produced by translation of RNA or the corresponding RNA is produced by transcription of a heterologous nucleic acid. Further, a protein is also heterologous to a host cell if it is a mutated version of an endogenous protein, and the mutation was introduced by genetic engineering.

The term “homologous”, as well as variations thereof, such as “homology”, refers to the similarity of a nucleic acid or amino acid sequence, typically in the context of a coding sequence for a gene or the amino acid sequence of a protein. Homology searches can be employed using a known amino acid or coding sequence (the “reference sequence”) for a useful protein to identify homologous coding sequences or proteins that have similar sequences and thus are likely to perform the same useful function as the protein defined by the reference sequence. A protein having homology to a reference protein is determined, for example and without limitation, by a BLAST (https://blast.ncbi.nlm.nih.gov) search. A protein with high percent homology is highly likely to carry out the identical biochemical reaction as the reference protein. In some cases, two enzymes having greater than 40% identity will carry out identical biochemical reactions, and the higher the identity, i.e., 40, 50%, 60%, 70%, 80%, 90% or greater than 95% identity, the more likely the two proteins have the same or similar function. A protein with at least 60% homology, and in some cases, at least 40% homology, to its reference protein is defined as substantially homologous. Any protein substantially homologous to a reference sequence can be used in a host cell according to the present disclosure.

Generally, homologous proteins share substantial sequence identity. Sets of homologous proteins generally possess one or more specific amino acids that are conserved across all members of the consensus sequence protein class. The percent sequence identity of a protein relative to a consensus sequence is determined by aligning the protein sequence against the consensus sequence. Practitioners in the art will recognized that various sequence alignment algorithms are suitable for aligning a protein with a consensus sequence. See, for example, Needleman, SB, et al., “A general method applicable to the search for similarities in the amino acid sequence of two proteins.” Journal of Molecular Biology 48 (3): 443-53 (1970). Following alignment of the protein sequence relative to the consensus sequence, the percentage of positions where the protein possesses an amino acid described by the same position in the consensus sequence determines the percent sequence identity. When a degenerate amino acid is present (i.e., B, Z, X, J or “+”) in a consensus sequence, any of the amino acids described by the degenerate amino acid may be present in the protein at the aligned position for the protein to be identical to the consensus sequence at the aligned position. When it is not possible to distinguish between two closely related amino acids, the following one-letter symbol is used-“B” refers to aspartic acid or asparagine; “Z” refers to glutamine or glutamic acid; “J” refers to leucine or isoleucine; and “X” or “+” refers to any amino acid.

A dash (−) in a consensus sequence indicates that there is no amino acid at the specified position. A plus (+) in a consensus sequence indicates any amino acid may be present at the specified position. Thus, a plus in a consensus sequence herein indicates a position at which the amino acid is generally non-conserved; a homologous enzyme sequence, when aligned with the consensus sequence, can have any amino acid at the indicated “+” position.

The terms “bio-based” or “non-petrochemically derived” or “renewable” as used herein refer to an organic compound that is synthesized from biologically produced organic components by fermenting a microorganism. These compounds are distinguished from wholly petroleum-derived compounds or those entirely of fossil origin. A compound of renewable or non-petrochemical origin include carbon atoms that have a non-petrochemical origin. Such non-petrochemical (or bio based or renewable) compounds have a 14C amount substantially higher than zero, such as about 1 parts per trillion or more, because they are derived from photosynthesis based starting material, such as for example, glucose or another feedstock used in producing such a compound, such as glyceric acid. In certain embodiments, such non-petrochemical based compositions provided herein, contain, for example, and without limitation: glyceric acid and other compounds of formula (I) which are non-petrochemical based.

In addition to identification of useful enzymes by percent sequence identity with a given consensus sequence, enzymes useful in the compositions and methods provided herein can also be identified by the occurrence of highly conserved amino acid residues in the query protein sequence relative to a consensus sequence. For each consensus sequence provided herein, a number of highly conserved amino acid residues are described. Enzymes useful in the compositions and methods provided herein include those that comprise a substantial number, and sometimes all, of the highly conserved amino acids at positions aligning with the indicated residues in the consensus sequence. Those skilled in the art will recognize that, as with percent identity, the presence or absence of these highly conserved amino acids in a query protein sequence can be determined following alignment of the query protein sequence relative to a given consensus sequence and comparing the amino acid found in the query protein sequence that aligns with each highly conserved amino acid specified in the consensus sequence.

Proteins that share a specific function are not always defined or limited by percent sequence identity. In some cases, a protein with low percent sequence identity with a reference protein is able to carry out the identical biochemical reaction as the reference protein. Such proteins may share three-dimensional structure which enables shared specific functionality, but not necessarily sequence similarity. Such proteins may share an insufficient amount of sequence similarity to indicate that they are homologous via evolution from a common ancestor and would not be identified by a BLAST search or other sequence-based searches. Thus, in some embodiments of the present disclosure, homologous proteins comprise proteins that lack substantial sequence similarity but share substantial functional similarity and/or substantial structural similarity.

As used herein, the term “express”, when used in connection with a nucleic acid encoding an enzyme or an enzyme itself in a cell, means that the enzyme, which may be an endogenous or exogenous (heterologous) enzyme, is produced in the cell. The term “overexpress”, in these contexts, means that the enzyme is produced at a higher level, i.e., enzyme levels are increased, as compared to the wild-type, in the case of an endogenous enzyme. Overexpression of an enzyme can be achieved by increasing the strength or changing the type of the promoter used to drive expression of a coding sequence, increasing the strength of the ribosome binding site or Kozak sequence, increasing the stability of the mRNA transcript, altering the codon usage, increasing the stability of the enzyme, and the like.

The terms “expression vector” or “vector” refer to a nucleic acid and/or a composition comprising a nucleic acid that can be introduced into a host cell, for example, by transduction, transformation, or infection, such that the cell then produces (i.e., expresses) nucleic acids and/or proteins other than those native to the cell, or in a manner not native to the cell, that are contained in or encoded by the nucleic acid so introduced. Thus, an “expression vector” contains nucleic acids (ordinarily DNA) to be expressed by the host cell. Optionally, the expression vector can be contained in materials to aid in achieving entry of the nucleic acids into the host cell, such as the materials associated with a virus, liposome, protein coating, or the like. Expression vectors suitable for use in various aspects and embodiments of the present disclosure include those into which a nucleic acid sequence can be, or has been, inserted, along with any operational elements. Thus, an expression vector can be transferred into a host cell and, typically, replicated therein (although, on can also employ, in some embodiments, non-replicable vectors that provide for “transient” expression). In some embodiments, an expression vector that integrates into chromosomal, mitochondrial, or plastid DNA is employed. In other embodiments, an expression vector that replicates extrachromasomally is employed. Typical expression vectors include plasmids, and expression vectors typically contain the operational elements for transcription of a nucleic acid in the vector. Such plasmids, as well as other expression vectors, are described herein or are well known to those of ordinary skill in the art.

The terms “ferment”, “fermentative”, and “fermentation” are used herein to describe culturing microbes under conditions to produce useful chemicals, including but not limited to conditions under which microbial growth, be it aerobic or anaerobic, occurs.

The terms “recombinant host cell”, “recombinant host microorganism”, and “strain” are used interchangeably herein to refer to a living cell that can be (or has been) transformed via insertion of an expression vector. A recombinant host cell or microorganism as described herein may be a prokaryotic cell (for example, a microorganism of the kingdom Eubacteria) or a eukaryotic cell. A prokaryotic cell lacks a membrane-bound nucleus, while a eukaryotic cell has a membrane-bound nucleus.

The terms “isolated” or “pure” refer to material that is substantially, for example, greater than 50%, 75%, 90%, 95%, 98% or 99%, free of components that normally accompany it in its native state, for example, the state in which it is naturally found or the state in which it exists when it is first produced. Additionally, any reference to a “purified” material is intended to refer to an isolated or pure material.

As used herein, the term “nucleic acid” and variations thereof shall be generic to polydeoxyribonucleotides (containing 2-deoxy-D-ribose), polyribonucleotides (containing D-ribose), segments of polydeoxyribonucleotides, and segments of polyribonucleotides. “Nucleic acid” can also refer to any other type of polynucleotide that is an N-glycoside of a purine or pyrimidine base, and to other polymers containing nonnucleotidic backbones, provided that the polymers contain nucleobases in a configuration that allows for base pairing and base stacking, as found in DNA and RNA. As used herein, the symbols for nucleotides and polynucleotides are those recommended by the IUPAC-IUB Commission of Biochemical Nomenclature (Biochem. 9:4022, 1970). A “nucleic acid” may also be referred to herein with respect to its sequence, the order in which different nucleotides occur in the nucleic acid, as the sequence of nucleotides in a nucleic acid typically defines its biological activity, for example, as in the sequence of a coding region, the nucleic acid in a gene composed of a promoter and coding region, which encodes the product of a gene, which may be an RNA, for example, a rRNA, tRNA, or mRNA, or a protein (where a gene encodes a protein, both the mRNA and the protein are “gene products” of that gene).

In the present disclosure, the term “genetic disruption” refers to several ways of altering genomic, chromosomal or plasmid-based gene expression. Non-limiting examples of genetic disruptions comprise CRISPR, RNAi, nucleic acid deletions, nucleic acid insertions, nucleic acid substitutions, nucleic acid mutations, knockouts, premature stop codons and transcriptional promoter modifications. In the present disclosure, “genetic disruption” is used interchangeably with “genetic modification”, “genetic mutation” and “genetic alteration.” Genetic disruptions give rise to altered gene expression and or altered protein activity. Altered gene expression encompasses decreased, eliminated and increased gene expression levels. In some examples, gene expression results in protein expression, in which case the term “gene expression” is synonymous with “protein expression”.

As used herein, “recombinant” refers to the alteration of genetic material by human intervention. Typically, recombinant refers to the manipulation of DNA or RNA in a cell or virus or expression vector by molecular biology (recombinant DNA technology) methods, including cloning and recombination. Recombinant can also refer to manipulation of DNA or RNA in a cell or virus by random or directed mutagenesis. A “recombinant” cell or nucleic acid can typically be described with reference to how it differs from a naturally occurring counterpart (the “wild-type”). In addition, any reference to a cell or nucleic acid that has been “engineered” or “modified” and variations of those terms, is intended to refer to a recombinant cell or nucleic acid.

The terms “transduce”, “transform”, “transfect”, and variations thereof as used herein refers to the introduction of one or more nucleic acids into a cell. For practical purposes, the nucleic acid is stably maintained or replicated by the cell for a sufficient period of time to enable the function(s) or product(s) it encodes to be expressed for the cell to be referred to as “transduced”, “transformed”, or “transfected”. Stable maintenance or replication of a nucleic acid may take place either by incorporation of the sequence of nucleic acids into the cellular chromosomal DNA, for example, the genome, as occurs by chromosomal integration, or by replication extrachromosomally, as occurs with a freely-replicating plasmid. A virus can be stably maintained or replicated when it is “infective”: when it transduces a host microorganism, replicates, and (without the benefit of any complementary virus or vector) spreads progeny expression vectors, for example, viruses, of the same type as the original transducing expression vector to other microorganisms, wherein the progeny expression vectors possess the same ability to reproduce.

As used herein, “glyceric acid” is intended to mean the molecule having the chemical formula C₃H₆O₄ and a molecular mass of 106.077 g/mol (CAS No. 473-81-4). The terms “glyceric acid” and “2,3-dihydroxypropanoic acid” are used interchangeably in the present disclosure, and are synonyms.

In conditions with pH values higher than the pKa of glyceric acid (for example, pH>3.52), glyceric acid is deprotonated to the glycerate anion C₃H₅O₄. Herein, “glycerate” is also used interchangeably with “glycerate anion”, and “2,3-dihydroxypropanoate”, and are synonyms.

Further, the glycerate anion is capable of forming an ionic bond with a cation to produce an glycerate salt. The term “glycerate” is intended to mean a variety of glycerate salt forms, and is used interchangeably with “glycerate salt”. Non-limiting examples of glycerates comprise sodium glycerate (CAS No. 50976-28-8), potassium glycerate (CAS No. 43110-90-3), and calcium glycerate (CAS No. 6057-35-8).

Glycerate salts can crystallize in various states of hydration and glycerate salts of the present disclosure are no exception. For example, calcium glycerate can form monohydrate crystals, wherein a single molecule of water crystallizes with a single molecule of calcium glycerate. Similarly, dihydrate crystals comprise two molecules of water for every molecule of calcium glycerate.

In conditions with pH values lower than the pKa of glyceric acid (for example, pH<3.52), the glycerate anion is protonated to form glyceric acid. Herein, “glycerate” is also used interchangeably with “glyceric acid” and are synonyms.

As used herein, “glycerate ester” is intended to mean an ester derived from glyceric acid, and it is synonymous with “alkyl glycerate”. Non-limiting examples of glycerate esters comprise methyl glycerate (molecular formula C₄H₈O₄ and average mass 120.104 Da), and ethyl glycerate (molecular formula (HO)₂C₂H₃COOC₂H₅).

The glyceric acid, glycerate salts, and glycerate esters of the present disclosure are synthesized from biologically produced organic components by a fermenting microorganism. For example, glyceric acid, glycerate salts, and glycerate esters are synthesized from the fermentation of sugars by recombinant host cells of the present disclosure. The prefix “bio-” or the adjective “bio-based” may be used to distinguish these biologically-produced glyceric acid, glycerate salts, and glycerate esters from those that are derived from petroleum feedstocks. As used herein, “glyceric acid” is defined as “bio-based glyceric acid”, “glycerate salt” is defined as “bio-based glycerate salt”, and “glycerate ester” is defined as “bio-based glycerate ester”.

As used herein, “glycolic acid” is intended to mean the molecule having the chemical formula C₂H₄O₃ and a molecular mass of 76.05 g/mol (CAS No. 79-14-1). The terms “glycolic acid”, “hydroxyacetic acid”, and “2-hydroxyacetic acid” are used interchangeably in the present disclosure, and are synonyms.

In conditions with pH values higher than the pKa of glycolic acid (for example, about pH>3.83 when using a sodium base, such as sodium hydroxide), glycolic acid is deprotonated to the glycolate/glycollate anion C₂H₃O₃⁻. Herein, “glycolate” is also used interchangeably with “glycollate”, “glycolate anion,” “glycollate anion”, “hydroxyacetate”, and “2-hydroxyacetate”, and are synonyms.

Further, the glycolate anion is capable of forming an ionic bond with a cation to produce an glycolate salt. The term “glycolate” is intended to mean a variety of glycolate salt forms, and is used interchangeably with “glycolate salt”. Non-limiting examples of glycolates comprise sodium glycolate (CAS No. 2836-32-0), calcium glycolate (CAS No. 996-23-6), potassium glycolate (CAS No. 25904-89-6), ethyl glycolate (CAS No. 623-50-7), and methyl glycolate (CAS No. 96-35-5).

In conditions with pH values lower than the pKa of glycolate acid (for example, pH<3.83), the glycolate anion is protonated to form glycolic acid. Herein, “glycolate” is also used interchangeably with “glycolic acid” and are synonyms.

As used herein, “glycolate ester” is intended to mean an ester derived from glycolic acid, and is synonymous with “alkyl glycolate”. Non-limiting examples of glycolate esters comprise ethyl glycolate (CAS No. 623-50-7), methyl glycolate (CAS No. 96-35-5) and benzyl glycolate (CAS No. 30379-58-9).

The glycolic acid, glycolate salts and glycolate esters of the present disclosure are synthesized from biologically produced organic components by a fermenting microorganism. For example, glycolic acid, glycolate salts, glycolate esters, or their precursor(s) are synthesized from the fermentation of sugars by recombinant host cells of the present disclosure. The prefix “bio-” or the adjective “bio-based” may be used to distinguish these biologically-produced glycolic acid and glycolates from those that are derived from petroleum feedstocks. As used herein, “glycolic acid” is defined as “bio-based glycolic acid”, “glycolate salt” is defined as “bio-based glycolate salt”, and “glycolate ester” is defined as “bio-based glycolate ester”.

As used herein, “acrylic acid” is intended to mean the molecule having the chemical formula C₃H₄O₂ and a molecular mass of 72.06 g/mol (CAS No. 79-10-7). The terms “acrylic acid”, “ethylene carboxylic acid”, “propenoic acid”, and “acroleic acid” are used interchangeably in the present disclosure, and are synonyms.

In conditions with pH values higher than the pKa of acrylic acid (for example, about pH>4.25 when using a sodium base, such as sodium hydroxide), acrylic acid is deprotonated to the acrylate anion C₃H₃O₂. Herein, “acrylate” is also used interchangeably with “propenoate”, and are synonyms.

Further, the acrylate anion is capable of forming an ionic bond with a cation to produce an acrylate salt. The term “acrylate” is intended to mean a variety of acrylate salt forms, and is used interchangeably with “acrylate salt”. Non-limiting examples of acrylates comprise sodium acrylate (CAS No. 7446-81-3), calcium acrylate (CAS No.6292-01-9), calcium diacrylate (CAS No. 6292-01-9), potassium acrylate (CAS No. 10192-85-5), ammonium acrylate (CAS No. 9003-03-6), sodium methacrylate (CAS No. 5536-61-8), and zinc acrylate (CAS No. 14643-87-9).

In conditions with pH values lower than the pKa of acrylic acid (for example, pH <4.25), the acrylate anion is protonated to form acrylic acid. Herein, “acrylate” is also used interchangeably with “acrylic acid” and are synonyms.

As used herein, “acrylate ester” is intended to mean an ester derived from acrylic acid, and is synonymous with “alkyl acrylate”. Non-limiting examples of acrylate esters comprise methyl acrylate (CAS No. 96-33-3), ethyl acrylate (CAS No. 140-88-5), butyl acrylate (CAS No. 141-32-2) and 2-ethylhexyl acrylate (2EHA; CAS No. 103-11-7).

The term “byproduct” or “by-product” means an undesired product related to the production of a target molecule. In the present disclosure, “byproduct” is intended to mean any amino acid, amino acid precursor, chemical, chemical precursor, organic acid, organic acid precursor, ester, ester precursor, biofuel, biofuel precursor, metabolite, or small molecule, that may accumulate during biosynthesis or chemical synthesis of glyceric acid, glycolic acid, acrylic acid, glycerate, glycolate, acrylate, glycerate ester, glycolate ester, acrylate ester, or other downstream product of the present disclosure. In some cases, “byproduct” accumulation may decrease the yields, titers or productivities of the target product (i.e., glyceric acid, glycolic acid, acrylic acid, glycerate, glycolate, acrylate, glycerate ester, glycolate ester, acrylate ester, or other downstream product) in a fermentation or in synthesis.

The redox cofactor nicotinamide adenine dinucleotide, NAD, comes in two forms—phosphorylated and un-phosphorylated. The term NAD (P) refers to both phosphorylated (NADP) and un-phosphorylated (NAD) forms, and encompasses oxidized versions (NAD⁺ and NADP) and reduced versions (NADH and NADPH) of both forms. The term “NAD(P)⁺” refers to the oxidized versions of phosphorylated and un-phosphorylated NAD, i.e., NAD⁺ and NADP⁺. Similarly, the term “NAD(P)H” refers to the reduced versions of phosphorylated and un-phosphorylated NAD, i.e., NADH and NADPH. When NAD(P)H is used to describe the redox cofactor in an enzyme catalyzed reaction, it indicates that NADH and/or NADPH is used. Similarly, when NAD(P)⁺ is the notation used, it indicates that NAD⁺ and/or NADP⁺ is used. While many proteins may bind either a phosphorylated or un-phosphorylated cofactor, there are redox cofactor promiscuous proteins, natural or engineered, that are indiscriminate; in these cases, the protein may use either NADH and/or NADPH. In some embodiments, enzymes that preferentially utilize either NAD (P) or NAD may carry out the same catalytic reaction when bound to either form.

Various values for temperatures, titers, yields, oxygen uptake rate (OUR), and pH are recited in the description and in the claims. It should be understood that these values are not exact. However, the values can be approximated to the rightmost/last/least significant figure, except where otherwise indicated. For example, a temperature range of from about 30° C. to about 42° C. covers the range 25° C. to 44° C. It should be understood that numerical ranges recited can also include the recited minimum value and the recited maximum value when the values are approximated to the rightmost/last/least significant figure. For example, a temperature range of from about 25° C. to about 50° C. covers the range of 25° C. to 50° C.

### Section 2: Recombinant Host Cells for Production of Glyceric Acid and Downstream Products 2.1 Host Cells

The present disclosure provides recombinant host cells engineered to produce glyceric acid, wherein the recombinant host cells comprise one or more heterologous nucleic acids encoding one or more glyceric acid pathway enzymes. In certain embodiments, the recombinant host cells further comprise one or more heterologous nucleic acids encoding one or more ancillary gene products (i.e., gene products other than the glyceric acid pathway enzymes) that improve yields, titers and/or productivities of glyceric acid. In particular embodiments, the recombinant host cells further comprise disruptions or deletions of endogenous nucleic acids that improve yields, titers and/or productivities of glyceric acid. In some embodiments, the recombinant host cells are capable of producing glyceric acid under aerobic conditions. In some embodiments, the recombinant host cells are capable of producing glyceric acid under substantially anaerobic conditions. In some embodiments, the recombinant host cells produce glyceric acid at increased titers, yields and productivities as compared to a parental host cell that does not comprise said heterologous nucleic acids.

In a first aspect, this disclosure provides recombinant host cells capable of producing glyceric acid comprising one or more heterologous nucleic acids that encode the glyceric acid biosynthetic pathway, wherein the pathway enzymes comprise a 3-phosphoglycerate phosphatase and/or a 2-phosphoglycerate phosphatase. In some embodiments, the recombinant host cells comprise heterologous nucleic acids encoding a 3-phosphoglycerate phosphatase with at least 60% amino acid identity with SEQ ID NO: 7 or SEQ ID NO: 9.

In a second aspect, this disclosure provides recombinant host cells that further comprise one or more heterologous nucleic acids encoding one or more ancillary proteins that function in redox cofactor recycling, redox cofactor biogenesis, or organic acid transport. In some embodiments, the one or more ancillary proteins comprise mitochondrial external NADH dehydrogenase, water-forming NADH oxidase, glyceric acid transporter, or combination thereof. In some embodiments, the ancillary protein has at least 60% amino acid identity with SEQ ID NO: 20, SEQ ID NO: 21, SEQ ID NO: 22, SEQ ID NO: 23, SEQ ID NO: 24, or SEQ ID NO: 2.

In a third aspect, this disclosure provides recombinant host cells that further comprise a genetic disruption of one or more genes wherein the one or more genes encodes phosphoglycerate mutase, phosphoglycerate dehydrogenase, enolase, glycerate 3-kinase, glycerate 2-kinase, or glycerol-3-phosphate dehydrogenase, or combination thereof. In some embodiments, the one or more genes has at least 60% amino acid identity with SEQ ID NO: 14, SEQ ID NO: 15, SEQ ID NO: 16, SEQ ID NO: 17, SEQ ID NO: 19, or SEQ ID NO: 25. In some of these embodiments, the recombinant host cells produce less than 5 g/l serine. In some of these embodiments, the recombinant host cells produce less than 5 g/l glycerol. In some of these embodiments, the recombinant host cells produce less than 5 g/l ethanol. In some of these embodiments, the recombinant host cells produce less than 5 g/l acetate.

In a fourth aspect, this disclosure provides recombinant host cells that further comprise one or more heterologous nucleic acids that encode a glycolic acid biosynthetic pathway, wherein the pathway enzymes comprise a hydroxypyruvate reductase, a hydroxypyruvate decarboxylase, and a glycoaldehyde dehydrogenase.

In a fifth aspect, this disclosure provides a method for the production of glyceric acid that comprises culturing the recombinant host cells of this disclosure for a sufficient period of time to produce glyceric acid. In some embodiments, the method further comprises an oxygen transfer rate greater than 10 mmol/l/hr. In some embodiments, the method further comprises an operational temperature of between about 25° C. and about 45° C. In some embodiments, the method further comprises a final fermentation broth pH of between about 2.5 and about pH 6. In some embodiments, the method produces a solution containing at least 50 g/l glyceric acid. In some embodiments, the method further comprises providing at least 100 g/l glucose to the recombinant host cell and producing an glyceric acid yield of at least 25%.

In a sixth aspect, this disclosure provides a method for the production of glycolic acid that comprises culturing the recombinant host cells of this disclosure for a sufficient period of time to produce glycolic acid. In some embodiments, the method further comprises an oxygen transfer rate greater than 10 mmol/l/hr. In some embodiments, the method further comprises an operational temperature of between about 25° C. and about 45° C. In some embodiments, the method further comprises a final fermentation broth pH of between about 2.5 and about pH 6. In some embodiments, the method produces a solution containing at least 50 g/l glycolic acid. In some embodiments, the method further comprises providing at least 100 g/l glucose to the recombinant host cell and producing a glycolic acid yield of at least 25%.

In a seventh aspect, this disclosure provides a method to produce glycerate esters comprising the steps of recovering glyceric acid or glycerate salt from fermentation broth, forming the glycerate ester by esterification with supercritical CO₂, and purifying said glycerate ester. In various embodiments, the esterification alcohol is methanol, ethanol, isopropyl alcohol, butanol, 3-pentanol, or isobutanol. In various embodiments, the esterification catalyst is H₂SO₄. In various embodiments, the glycerate ester is methyl glycerate, ethyl glycerate, butyl glycerate, and 2-ethylhexyl glycerate.

In an eighth aspect, this disclosure provides a deoxydehydration method to produce acrylate esters comprising the steps of converting a glycerate ester to an acrylate ester, and purifying said acrylate ester. In various embodiments, the deoxydehydration alcohol is methanol, ethanol, isopropyl alcohol, butanol, 3-pentanol, or isobutanol. In various embodiments, the deoxydehydration catalyst is selected from the non-limiting group comprising (NH₄)₆Mo₇O₂₄·4H₂O, MoO₂Cl₂, MoO₂(CH₃)₂, MoO₂Cl₂, MoO₂Br₂, (Bu₄N)₂Mo₆O₁₉, HReO₄, Re₂(CO)₁₀, BrRe(CO)₅, (Cp*Ru(CO)₂)₂, or CH₃ReO₃. In various embodiments, the deoxyhydration reducing agent is selected from the non-limiting group comprising PPh₃, H₂, Na₂SO₃, benzene, toluene, 5-nonanol, 3-octanol, 2-octanol, 1-butanol, 3-pentanol, 2-methyl-1-butanol, 2-ethylhaxanol, or isopropanol. In various embodiments, the acrylate ester is methyl acrylate, ethyl acrylate, butyl acrylate, and 2-ethylhexyl acrylate.

In a ninth aspect, provided herein is a poly (glycerate carbonate) compound comprising repeat units of formula (I) or a salt thereof:

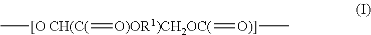

wherein each R¹ is independently hydrogen, optionally substituted C1-C5 alkyl, and optionally substituted aryl.

In another aspect, provided herein is a composition comprising a compound prepared comprising the method provided herein.

In another embodiment, the composition provided herein contains non-petrochemical based components, or substantially contains non-petrochemical based components, such as about 25% or more, about 50% or more, about 75% or more, or about 90% or more non-petrochemical based components.

In another aspect, provided herein is an article of manufacture comprising a compound prepared comprising the method provided herein. In another aspect, provided herein is an article of manufacture comprising a compound provided herein. In one embodiment, the article of manufacture is a film. In one embodiment, the article of manufacture is a fiber. In one embodiment, the article of manufacture is a hydrogel. In another aspect, the article is degradable through hydrolytic and biological processes allow stimuli-responsive behavior.

In some embodiments, the recombinant host cells comprise one or more heterologous nucleic acids encoding one or more downstream pathway enzymes that use glycerate as a precursor, substrate, metabolic intermediate, or building block in the production of a downstream product. Non-limiting examples of a downstream product comprise glyceric acid, glycolic acid, acrylic acid, glycerate, glycolate, acrylate, glycerate ester, glycolate ester, acrylate ester, and any molecule that uses glycerate as a precursor, substrate, metabolic intermediate or building block in its synthesis. In some embodiments, the downstream product is an organic acid. In some embodiments, the recombinant host cells produce a downstream product at increased titers, yields and productivities as compared to a parental host cell that does not comprise said heterologous nucleic acids.

In some embodiments, the recombinant host cells further comprise one or more heterologous nucleic acids encoding one or more ancillary gene products (i.e., gene products other than the downstream product pathway enzymes) that improve yields, titers and/or productivities of a downstream product. In particular embodiments, the recombinant host cells further comprise disruptions or deletions of endogenous nucleic acids that improve yields, titers and/or productivities of a downstream product. In some embodiments, the recombinant host cells are capable of producing a downstream product under aerobic conditions. In some embodiments, the recombinant host cells are capable of producing a downstream product under substantially anaerobic conditions.

Any suitable host cell may be used in practice of the methods of the present disclosure, and examples of host cells useful in the compositions and methods provided herein include archaeal, prokaryotic, or eukaryotic cells. In an embodiment of the present disclosure, the recombinant host cell is a prokaryotic cell. In an embodiment of the present disclosure, the recombinant host cell is a eukaryotic cell. In an embodiment of the present disclosure, the recombinant host cell is a Pichia kudriavzevii (P. kudriavzevii) strain. Methods of construction and genotypes of these recombinant host cells are described herein.

In some embodiments, the recombinant host cell is a yeast cell. In certain embodiments, the yeast cell belongs to the Issatchenkia orientalis/Pichia fermentans clade. In some embodiments, the yeast cell belongs to the genus Pichia, Issatchenkia or Candida. In some embodiments, the yeast cell is Pichia kudriavzevii. In some embodiments, the yeast cell belongs to the Saccharomyces clade. In some embodiments, the yeast cell is Saccharomyces cerevisiae. In other embodiments, the recombinant host cell is a prokaryotic cell. In some embodiments, the prokaryotic cell belongs to the genus Escherichia, Corynebacterium, Bacillus, or Lactococcus. In some embodiments, the prokaryotic cell is Escherichia coli, Corynebacterium glutamicum, Bacillus subtilis, or Lactococcus lactis.

### 2.1.1 Yeast Cells

In an embodiment of the present disclosure, the recombinant host cell is a yeast cell. Yeast cells are excellent host cells for construction of recombinant metabolic pathways comprising heterologous enzymes catalyzing production of small-molecule products. There are established molecular biology techniques and nucleic acids encoding genetic elements necessary for construction of yeast expression vectors, including, but not limited to, promoters, origins of replication, antibiotic resistance markers, auxotrophic markers, terminators, and the like. Second, techniques for integration/insertion of nucleic acids into the yeast chromosome by homologous recombination are well established. Yeast also offers a number of advantages as an industrial fermentation host. Yeast cells can generally tolerate high concentrations of organic acids and maintain cell viability at low pH and can grow under both aerobic and anaerobic culture conditions, and there are established fermentation broths and fermentation protocols. This characteristic results in efficient product biosynthesis when the host cell is supplied with a carbohydrate carbon source. Also, from a process standpoint, the ability to run fermentations under substantially anaerobic conditions can decrease production cost.

In various embodiments, yeast cells useful in the methods of the present disclosure include yeasts of the genera Aciculoconidium, Ambrosiozyma, Arthroascus, Arxiozyma, Ashbya, Babjevia, Bensingtonia, Botryoascus, Botryozyma, Brettanomyces, Bullera, Bulleromyces, Candida, Citeromyces, Clavispora, Cryptococcus, Cystofilobasidium, Debaryomyces, Dekkara, Dipodascopsis, Dipodascus, Eeniella, Endomycopsella, Eremascus, Eremothecium, Erythrobasidium, Fellomyces, Filobasidium, Galactomyces, Geotrichum, Guilliermondella, Hanseniaspora, Hansenula, Hasegawaea, Holtermannia, Hormoascus, Hyphopichia, Issatchenkia, Kloeckera, Kloeckeraspora, Kluyveromyces, Kondoa, Kuraishia, Kurtzmanomyces, Leucosporidium, Lipomyces, Lodderomyces, Malassezia, Metschnikowia, Mrakia, Myxozyma, Nadsonia, Nakazawaea, Nematospora, Ogataea, Oosporidium, Pachysolen, Phachytichospora, Phaffia, Pichia, Rhodosporidium, Rhodotorula, Saccharomyces, Saccharomycodes, Saccharomycopsis, Saitoella, Sakaguchia, Saturnospora, Schizoblastosporion, Schizosaccharomyces, Schwanniomyces, Sporidiobolus, Sporobolomyces, Sporopachydermia, Stephanoascus, Sterigmatomyces, Sterigmatosporidium, Symbiotaphrina, Sympodiomyces, Sympodiomycopsis, Torulaspora, Trichosporiella, Trichosporon, Trigonopsis, Tsuchiyaea, Udeniomyces, Waltomyces, Wickerhamia, Wickerhamiella, Williopsis, Yamadazyma, Yarrowia, Zygoascus, Zygosaccharomyces, Zygowilliopsis, and Zygozyma, among others.

In various embodiments, the yeast cell is of a species selected from the non-limiting group comprising Candida albicans, Candida ethanolica, Candida guilliermondii, Candida krusei, Candida lipolytica, Candida methanosorbosa, Candida sonorensis, Candida tropicalis, Candida utilis, Cryptococcus curvatus, Hansemila polymorpha, Issatchenkia orientalis, Kluyveromyces lactis, Kluyveromyces marxianus, Kluyveromyces thermotolerans, Komagataella pastoris, Lipomyces starkeyi, Pichia angusta, Pichia deserticola, Pichia galeiformis, Pichia kodamae, Pichia kudriavzevii (P. kudriavzevii), Pichia membranaefaciens, Pichia methanolica, Pichia pastoris, Pichia salicaria, Pichia stipitis, Pichia thermotolerans, Pichia trehalophila, Rhodosporidium toruloides, Rhodotorula glutinis, Rhodotorula graminis, Saccharomyces bayanus, Saccharomyces boulardi, Saccharomyces cerevisiae (S. cerevisiae), Saccharomyces kluyveri, Schizosaccharomyces pombe (S. pombe) and Yarrowia lipolytica. This list encompasses yeast in the broadest sense.

The Crabtree phenomenon refers to the capability of yeast cells to convert glucose to alcohol in the presence of high sugar concentrations and oxygen instead of producing biomass via the tricarboxylic acid (TCA) cycle. Yeast cells produce alcohol to prevent growth of competing microorganisms in high sugar environments, which yeast cells can utilize later on when the sugars are depleted. Many yeast can typically use two pathways to produce ATP from sugars: the first involves the conversion of a sugars (via pyruvate) to carbon dioxide via the TCA cycle, and the second involves the conversion of sugars (via pyruvate) to ethanol. Yeast cells that display a Crabtree effect (known as Crabtree-positive yeast cells) are able to simultaneously use both pathways. Yeast cells that do not display a Crabtree effect (known as Crabtree-negative yeast cells) only convert pyruvate to ethanol when oxygen is absent. In some embodiments of the present disclosure, the host cell is a Crabtree-positive yeast cell. In other embodiments, the host cell is a Crabtree-negative yeast cell. In certain embodiments, the host cell displays a phenotype along a continuum of traits between Crabtree-positive and Crabtree-negative and is thus neither exclusively a Crabtree-positive yeast cell nor Crabtree negative yeast cell. It is advantageous to use a Crabtree-negative yeast or a yeast with perceptible Crabtree-negative tendencies or traits to produce glyceric acid because high glucose concentrations can be maintained during product biosynthesis without ethanol accumulation; ethanol is an undesired byproduct in glyceric acid production. P. kudriavzevii does not produce appreciable amounts of ethanol from pyruvate at high glucose concentrations in the presence of oxygen, and as such is a Crabtree-negative yeast. In some embodiments, the host cell is P. kudriavzevii.

In certain embodiments, the recombinant yeast cells provided herein are engineered by the introduction of one or more genetic modifications (including, for example, heterologous nucleic acids encoding enzymes and/or the disruption of native nucleic acids encoding enzymes) into a Crabtree-negative yeast cell. In certain of these embodiments, the host cell belongs to the Pichia/Issatchenkia/Saturnispora/Dekkera clade. In certain of these embodiments, the host cell belongs to the genus selected from the group comprising Pichia, Issatchenkia, or Candida. In certain embodiments, the host cell belongs to the genus Pichia, and in some of these embodiments the host cell is P. kudriavzevii. Members of the Pichia/Issatchenkia/Saturnispora/Dekkera or the Saccharomyces clade are identified by analysis of their 26S ribosomal DNA using the methods described by Kurtzman C. P., and Robnett C. J., (“Identification and Phylogeny of Ascomycetous Yeasts from Analysis of Nuclear Large Subunit (26S) Ribosomal DNA Partial Sequences”, Atonie van Leeuwenhoek 73 (4): 331-371; 1998). Kurtzman and Robnett report analysis of approximately 500 ascomycetous yeasts were analyzed for the extent of divergence in the variable D1/D2 domain of the large subunit (26S) ribosomal DNA. Host cells encompassed by a clade exhibit greater sequence identity in the D1/D2 domain of the 26S ribosomal subunit DNA to other host cells within the clade as compared to host cells outside the clade. Therefore, host cells that are members of a clade (for example, the Pichia/Issatchenkia/Saturnispora/Dekkera or Saccharomyces clades) can be identified using the methods of Kurtzman and Robnett.

In certain embodiments of the present disclosure, the recombinant host cells are engineered by introduction of one or more genetic modifications into a Crabtree-positive yeast cell. In certain of these embodiments, the host cell belongs to the Saccharomyces clad. In certain of these embodiments, the host cell belongs to a genus selected from the group comprising Saccharomyces, Schizosaccharomyces, Brettanomyces, Torulopsis, Nematospora and Nadsonia. In certain embodiments, the host cell belongs to the genus Saccharomyces, and in one of these embodiments the host cell is S. cerevisiae.

### 2.1.2 Eukaryotic Cells

In addition to yeast cells, other eukaryotic cells are also suitable for use in accordance with methods of the present disclosure, so long as the engineered host cell is capable of growth and/or product formation. Illustrative examples of eukaryotic host cells provided by the present disclosure include, but are not limited to cells belonging to the genera Aspergillus, Crypthecodinium, Cunninghamella, Entomophthora, Mortierella, Mucor, Neurospora, Pythium, Schizochytrium, Thraustochytrium, Trichoderma, and Xanthophyllomyces. Examples of eukaryotic strains include, but are not limited to: Aspergillus niger, Aspergillus oryzae, Crypthecodinium cohnii, Cunninghamella japonica, Entomophthora coronata, Mortierella alpina, Mucor circinelloides, Neurospora crassa, Pythium ultimum, Schizochytrium limacinum, Thraustochytrium aureum, Trichoderma reesei and Xanthophyllomyces dendrorhous.

### 2.1.3 Archaeal Cells

Archaeal cells are also suitable for use in accordance with methods of the present disclosure, and in an embodiment of the present disclosure, the recombinant host cell is an archaeal cell. Illustrative examples of recombinant archaea host cells provided by the present disclosure include, but are not limited to, cells belonging to the genera: Aeropyrum, Archaeglobus, Halobacterium, Methanococcus, Methanobacterium, Pyrococcus, Sulfolobus, and Thermoplasma. Examples of archaea strains include, but are not limited to Archaeoglobus fulgidus, Halobacterium sp., Methanococcus jannaschii, Methanobacterium thermoautotrophicum, Thermoplasma acidophilum, Thermoplasma volcanium, Pyrococcus horikoshii, Pyrococcus abyssi, and Aeropyrum pernix.

### 2.1.4 Prokaryotic Cells

In an embodiment of the present disclosure, the recombinant host cell is a prokaryotic cell. Prokaryotic cells are suitable host cells for construction of recombinant metabolic pathways comprising heterologous enzymes catalyzing production of small-molecule products. Illustrative examples of recombinant prokaryotic host cells include, but are not limited to, cells belonging to the genera Agrobacterium, Alicyclobacillus, Anabaena, Anacystis, Arthrobacter, Azobacter, Bacillus, Brevibacterium, Chromatium, Clostridium, Corynebacterium, Enterobacter, Erwinia, Escherichia, Lactobacillus, Lactococcus, Mesorhizobium, Methylobacterium, Microbacterium, Pantoea, Phormidium, Pseudomonas, Rhodobacter, Rhodopseudomonas, Rhodospirillum, Rhodococcus, Salmonella, Scenedesmun, Serratia, Shigella, Staphlococcus, Strepromyces, Synnecoccus, and Zymomonas. Examples of prokaryotic strains include, but are not limited to, Bacillus subtilis (B. subtilis), Brevibacterium ammoniagenes, Bacillus amyloliquefacines, Brevibacterium ammoniagenes, Brevibacterium immariophilum, Clostridium acetobutylicum, Clostridium beigerinckii, Corynebacterium glutamicum (C. glutamicum), Enterobacter sakazakii, Escherichia coli (E. coli), Lactobacillus acidophilus, Lactococcus lactis, Mesorhizobium loti, Pantoea ananatis (P. ananatis), Pseudomonas aeruginosa, Pseudomonas mevalonii, Pseudomonas pudita, Rhodobacter capsulatus, Rhodobacter sphaeroides, Rhodospirillum rubrum, Salmonella enterica, Salmonella typhi, Salmonella typhimurium, Shigella dysenteriae, Shigella flexneri, Shigella sonnei, and Staphylococcus aureus.

### 2.2 Glyceric Acid Pathway

Provided herein in certain embodiments are recombinant host cells having at least one active glyceric acid pathway from a glycolytic intermediate to glycerate. Recombinant host cells having an active glyceric acid pathway are used as described herein to produce one or more active enzymes necessary to catalyze each metabolic reaction in a glyceric acid pathway, and therefore are capable of producing glyceric acid in measurable yields and/or titers when cultured under suitable conditions. Recombinant host cells having a glyceric acid pathway comprise one or more heterologous nucleic acids encoding glyceric acid pathway enzyme(s) and are capable of producing glycerate. In some embodiments, the recombinant host cell is Pichia kudriavzevii.

Recombinant host cells may employ combinations of metabolic reactions for biosynthetically producing the compounds of the present disclosure. The biosynthesized compounds produced by the recombinant host cells comprise glycerate, glyceric acid, and the intermediates, products and derivatives of the glyceric acid pathway. The biosynthesized compounds can be produced intracellularly and/or secreted into the fermentation medium.

### 2.2.1 Glyceric Acid Pathway Enzymes

The glyceric acid pathway described herein produces glycerate from glucose with the following balanced, stoichiometric equation:

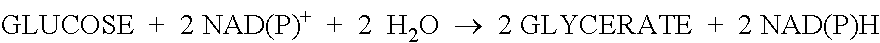

In many embodiments, recombinant host cells of the present disclosure comprise a glyceric acid pathway that proceeds via (1) 3-phosphoglycerate (3-PG; synonymous with glycerate-3-phosphate and 3-phosphoglyceric acid); or, (2) 2-phosphoglycerate (2-PG; synonymous with glycerate-2-phosphate and 2-phosphoglyceric acid); or, (3) 3-PG and 2-PG (Table 1). In some embodiments, the recombinant host cells comprise a glyceric acid pathway that proceeds via 3-PG, wherein a 3-PG phosphatase (EC #3.1.3.38) converts one molecule of 3-PG and one molecule of water into one molecule of glycerate and one molecule of orthophosphate. In other embodiments, the recombinant host cells comprise a glyceric acid pathway that proceeds via 2-PG, wherein a 2-PG phosphatase (EC #3.1.3.20) converts one molecule of 2-PG and one molecule of water into one molecule of glycerate and one molecule of orthophosphate. In some embodiments, the recombinant host cells comprise a glyceric acid pathway that proceeds via 2-PG. In some embodiments, the recombinant host cells comprise a glyceric acid pathway that proceeds via both 3-PG and 2-PG. In some embodiments, the recombinant host cells comprise a glyceric acid pathway with one or more enzymes that are derived from a phosphoglycerate monoester hydrolase (EC #3.1.3.X).

The advantaged thermodynamics of the pathway will help to achieve high glyceric acid yields, titers and/or productivities, and/or high downstream product yields, titers and/or productivities. The conversion of glucose to glycerate via the glyceric acid pathways described herein has a calculated change in Gibbs free energy of −73.8+/−3.4 KJ/mol (i.e., ΔᵣG^{m} calculated at 1 mM metabolite concentrations, 25° C., pH 7.0, and 0.1 M ionic strength; conditions typically observed in yeast), a negative value indicative of a strong driving force that pushes the reaction to completion, favoring product formation. Due to its mechanism of action, the 3-PG/2-PG phosphatase-catalyzed reaction should also be irreversible, although this disclosure is not bound by any particular method or mechanism. Additionally, net formation of NAD (P) H via said reaction can be used by recombinant host cells for ATP generation in the electron transport chain, thereby minimizing the amount of additional glucose used to support product export and cellular maintenance.

TABLE 1
Enzymes in a glycerate-producing reaction

| EC # | Enzyme name | Reaction catalyzed |
|---|---|---|
| 3.1.3.38 | 3-Phosphoglycerate | 3-Phosphoglycerate + H₂O → Glycerate + |
| | phosphatase | orthophosphate |
| 3.1.3.20 | 2-Phosphoglycerate | 2-Phosphoglycerate + H₂O → Glycerate + |
| | phosphatase | orthophosphate |
| 3.1.3.X | A phosphoglycerate | A phosphoglycerate monoester substrate |
| | monoester hydrolase | (with or without other reactants) → a |
| | | glycerate monoester + orthophosphate |
| | | (with or without other products) |

### 2.2.1.1 3-Phosphoglycerate Phosphatase

The 3-PG phosphatase (EC #3.1.3.38) described herein catalyzes a one-step reaction of the glyceric acid pathway, wherein one molecule of 3-PG and one molecule of water are converted to one molecule of glycerate and one molecule of orthophosphate (Table 1).

Any enzyme is suitable for use in accordance with this disclosure so long as the enzyme is capable of catalyzing said 3-PG phosphatase reaction described above. In an embodiment of the present disclosure, a glyceric acid pathway comprising a 3-PG phosphatase is calculated to thermodynamically favor the conversion of glucose to glycerate. In some embodiments, recombinant host cells comprise one or more heterologous nucleic acids encoding a 3-PG phosphatase wherein said recombinant host cells are capable of producing glycerate. In certain embodiments, recombinant host cells comprise one or more heterologous nucleic acids encoding a 3-PG phosphatase, wherein the heterologous nucleic acid is expressed in sufficient amounts to produce glycerate. In various embodiments, recombinant host cells may comprise multiple copies of a single heterologous nucleic acid and/or multiple copies of two or more heterologous nucleic acids. Recombinant host cells comprising multiple heterologous nucleic acids may comprise any number of heterologous nucleic acids. Although phosphatase enzymes are ubiquitous in nature, no enzyme is known to specifically catalyze this reaction. Some substrate-promiscuous phosphatases have been reported with activity towards 3-PG, but these enzymes also dephosphorylate a broad range of glycolytic intermediates and other phosphorylated compounds, and are either not specific enough or not active enough toward 3-PG. Further, not all phosphatases are substrate-promiscuous, and many substrate-specific phosphatases have also been characterized with good activity toward compounds structurally similar to 3-PG, including phosphoserine (EC #3.1.3.3), glycerol-3-phosphate (EC #3.1.3.21) and phosphoglycolate (EC #3.1.3.18). Thus, although any enzyme, particularly, any phosphoglycerate monoester hydrolase (EC #3.1.3.X), is suitable for use in accordance with this disclosure so long as the enzyme is capable of catalyzing said 3-PG phosphatase reaction, not any such natural enzyme is able to catalyze said reaction with sufficient specificity or efficiency to produce reasonable product yields and/or titers. In many embodiments, the 3-PG phosphatase has one or more active site mutations. In some embodiments, the one or more active site mutations lies in the active site, catalytic site, or substrate binding site.

Methods for identifying and improving enzymes towards desired 3-PG phosphatase activity are disclosed below in section 2.2.2 (on methods to identify and/or improve enzymes in the glyceric acid pathway). The methods described below for protein mutagenesis, identification, expression, purification, and characterization are methods widely-practiced by practitioners skilled in the art, who will appreciate that a wide variety of commercial solutions are available for such endeavors. Briefly, methods that may be used (as described in section 2.2.2) comprise random and rational mutagenesis methods such as site-specific mutagenesis, error-prone PCR, and directed evolution. Libraries of mutated genes or strains are generated, and mutagenized proteins are characterized, and integrated into the genome of recombinant host cells for further strain characterization. The methods disclosed may be adapted as needed depending on the target enzyme properties desired.

In some embodiments, the 3-PG phosphatase enables improved pathway flux. In some embodiments, the 3-PG phosphatase enables increased yield, titer and/or productivity. In some embodiments, the 3-PG phosphatase enables increased substrate specificity. In some embodiments, the 3-PG phosphatase displays improved kinetic properties, such as Kₘ and k_{cat}. In some embodiments, the 3-PG phosphatase is a product of one or more protein engineering cycles. In some embodiments, the 3-PG phosphatase comprises one or more point mutations. In some embodiments, the 3-PG phosphatase has improved Kₘ and/or k_{cat} for the substrate 3-PG. In some embodiments, the 3-PG phosphatase has Kₘ≤3 mM with 3-PG as substrate. In some embodiments, the 3-PG phosphatase has k_{cat}≥10 per second with 3-PG as substrate.

In some embodiments, the 3-PG phosphatase is derived from a phosphoserine phosphatase (EC #3.1.3.3), glycerol-1-phosphatase (EC #3.1.3.21), or phosphoglycolate phosphatase (EC #3.1.3.18).

In some embodiments, the 3-PG phosphatase is derived from a bacterial source. In many of these embodiments, the 3-PG phosphatase is derived from a cell belonging to a genus selected from the group comprising Escherichia, Mycobacterium, Haemophilus and Synechococcus. Non-limiting examples of bacterial enzymes comprise Escherichia coli UniProt ID: P32662, Mycobacterium avium UniProt ID: AOQJI1, Haemophilus somnus UniProt ID: Q011W8, Synechococcus elongatus UniProt ID: Q55320, and Escherichia coli UniProt ID: P27848.

In some embodiments, the 3-PG phosphatase is derived from the Escherichia coli phosphoglycolate phosphatase (abbv. EcGPH; UniProt ID: P32662; SEQ ID NO: 1). EcGPH catalyzes the conversion of one molecule of 2-phosphoglycolate (2P-Gly) and one molecule of water to one molecule of glycolate and one molecule of phosphate. 2P-Gly and 3-PG are structurally similar, making EcGPH a good starting point for engineering a 3-PG phosphatase. In various embodiments, the 3-PG phosphatase derived from a 2P-Gly phosphatase has one or more active site mutations resulting in increased enzyme activity and/or specificity toward 3-PG.

In some embodiments, the 3-PG phosphatase is derived from the Mycobacterium avium phosphoserine phosphatase (abbv. MaSERB; UniProt ID: AOQJI1; SEQ ID NO: 2; PDB: AOQJI1). MaSERB catalyzes the conversion of one molecule of O-phospho-L (or D)-serine and one molecule of water to one molecule of L (or D)-serine and one molecule of phosphate. O-phospho-L(or D)-serine and 3-PG are structurally similar, making MaSERB a good starting point for engineering a 3-PG phosphatase. In various embodiments, the 3-PG phosphatase derived from MaSERB has one or more active site mutations comprising E196S, T, N, Q, A, V, I or L; D189S, T, N, Q, A, V, I or L; or any combination thereof.

In some embodiments, the 3-PG phosphatase is derived from the Haemophilus somnus phosphoglycolate phosphatase (abbv. HsGPH; UniProt ID: Q011W8; SEQ ID NO: 3; PDB: 2HSZ). HsGPH catalyzes the conversion of one molecule of 2P-Gly and one molecule of water to one molecule of glycolate and one molecule of phosphate. In various embodiments, the 3-PG phosphatase derived from HsGPH has one or more active site mutations at the amino acid positions comprising A49, Q178, N179, N199, Y200, N201, or any combination thereof.

In some embodiments, the 3-PG phosphatase is derived from the Synechococcus elongates alkaline phosphatase (abbv. SePHOV; UniProt ID: Q55320, SEQ ID NO: 6). SePHOV catalyzes the conversion of phosphate monoester and water to alcohol and phosphate. In various embodiments, the 3-PG phosphatase is without an N-terminal signal sequence. In some embodiments, the 3-PG phosphatase is without its first 20 amino acids. In various embodiments, the 3-PG phosphatase has one or more active site mutations

In some embodiments, the 3-PG phosphatase is derived from the Escherichia coli phosphosugar phosphatase (abbv. EcYIGL; UniProt ID: P27848; SEQ ID NO: 8). EcYIGL catalyzes the conversion of one molecule of pyridoxal 5′-phosphate and one molecule of water to one molecule of pyridoxal and one molecule of phosphate. In various embodiments, the 3-PG phosphatase derived from EcYIGL has one or more active site mutations.

In some embodiments, the 3-PG phosphatase is derived from a eukaryotic source. In many of these embodiments, the 3-PG phosphatase is derived form a cell belonging to a genus selected from the group comprising Saccharomyces, Pichia, and Homo. In some embodiments, the recombinant host cell is Pichia kudriavzevii.

Non-limiting examples of eukaryotic enzymes comprise Saccharomyces cerevisiae UniProt ID: P40106, Saccharomyces cerevisiae UniProt ID: P19881, Saccharomyces cerevisiae UniProt ID: A6ZX98, Saccharomyces cerevisiae UniProt ID: G2WBT3, Saccharomyces cerevisiae UniProt ID: C7GJQ5, Saccharomyces cerevisiae UniProt ID: P36151, Pichia kudriavzevii UniProt ID: AOAIZ8JUP5, Pichia kudriavzevii UniProt ID: A0A099NZ38, Pichia kudriavzevii UniProt ID: A0A1Z8JN52, and Homo sapiens UniProt ID: A6NDG6.

In some embodiments, the 3-PG phosphatase is derived from the Saccharomyces cerevisiae glycerol-1-phosphate phosphatase (abbv. ScGPP2; UniProt ID: P40106; SEQ ID NO:

4). ScGPP2 catalyzes the conversion of one molecule of glycerol-1-phosphate and one molecule of water to one molecule of glycerol and one molecule of phosphate. In various embodiments, the 3-PG phosphatase derived from ScGPP2 has one or more active site mutations comprising N204R, H, K, Q, A or Y.

In some embodiments, the 3-PG phosphatase is derived from the Homo sapiens glycerol-3-phosphate phosphatase (abbv. HsPGP; UniProt ID: A6NDG6; SEQ ID NO: 5). HsPGP catalyzes the conversion of glycerol-3-phosphate and water to glycerol and phosphate. In various embodiments, the 3-PG phosphatase has one or more active site mutations.

In some embodiments, the 3-PG phosphatase is derived from Saccharomyces cerevisiae 4-nitrophenylphosphatase (abbv. ScPHO13; UniProt ID: P19881; SEQ ID NO: 9). ScPHO13 catalyzes the conversion of one molecule of 4-nitrophenyl phosphate and one molecule of water to one molecule of 4-nitrophenol and one molecule of phosphate. In some embodiments, the 3-PG phosphatase derived from ScPHO13 has one or more active site mutations.

In some embodiments, the 3-PG phosphatase is derived from Pichia kudriavzevii Pho13 protein (abbv. PkPHO13; UniProt ID: A0A1Z8JUP5; SEQ ID NO: 10). PKPHO13 catalyzes the conversion of one molecule of 4-nitrophenyl phosphate and one molecule of water to one molecule of 4-nitrophenol and one molecule of phosphate. In some embodiments, the 3-PG phosphatase derived from PkPHO13 has one or more active site mutations.

In some embodiments, the 3-PG phosphatase is derived from Pichia kudriavzevii ORF64 protein (abbv. PKORF64; UniProt ID: A0A099NZ38; SEQ ID NO: 11). In some embodiments, the 3-PG phosphatase derived from PKORF64 has one or more active site mutations.

In some embodiments, the 3-PG phosphatase is derived from Pichia kudriavzevii ORF423 protein (abbv. PKORF423; UniProt ID: A0A1Z8JN52; SEQ ID NO: 12). In some embodiments, the 3-PG phosphatase derived from PKORF423 has one or more active site mutations.

In some embodiments, the 3-PG phosphatase is derived from Saccharomyces cerevisiae uncharacterized protein YKR070W (abbv. ScYKR070W; UniProt ID: P36151; SEQ ID NO: 13). In some embodiments, the 3-PG phosphatase derived from PkORF423 has one or more active site mutations.

In some embodiments, the 3-PG phosphatase has a Km of less than about 5 mM with 3-PG as the substrate. In some embodiments, the 3-PG phosphatase has a Km for 3-PG that is less than Kₘ for other glycolytic intermediates. In some embodiments, the 3-PG phosphatase has a k_{cat} of greater than about 10 turnovers per second with 3-PG as the substrate. In some embodiments, the 3-PG phosphatase has a k_{cat} for 3-PG that is greater than the k_{cat} for other glycolytic intermediates. In some embodiments, the 3-PG phosphatase has an improved k_{cat}/Kₘ compared to the protein that it was derived from.

In many embodiments, recombinant host cells comprise one or more heterologous nucleic acids encoding a 3-PG phosphatase wherein said recombinant host cells are capable of producing glycerate. In various embodiments, proteins suitable for use in accordance with methods of the present disclosure have 3-PG phosphatase activity and comprise an amino acid sequence with at least 40%, at least 50%, at least 60%, at least 70%, at least 80%, at least 90%, or at least 95% sequence identity with SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, or SEQ ID NO: 13. In many embodiments, the recombinant host cell is a P. kudriavzevii strain.

The present disclosure also provides consensus sequences (defined above) useful in identifying and/or constructing glyceric acid pathway enzymes suitable for use in accordance with the methods of the present disclosure. In various embodiments, these consensus sequences comprise active site amino acid residues involved in substrate recognition and reaction catalysis, as described herein. Thus, an enzyme encompassed by a consensus sequence provided herein has an enzymatic activity that is identical, or essentially identical, or at least substantially similar with respect to ability to catalyze the reaction performed by one of the enzymes exemplified herein. For example, a 3-PG phosphatase encompassed by a 3-PG phosphatase consensus sequence provided herein has an enzymatic activity that is identical, or essentially identical, or at least substantially similar with respect to ability to convert one molecule of 3-PG and one molecule of water to one molecule of glycerate and one molecule of orthophosphate. As noted above, any protein substantially homologous to 3-PG phosphatase as described herein can be used in a recombinant host cell of the present disclosure.

The 3-PG phosphatase consensus sequence #1 (SEQ ID NO: 7) provides the sequence of amino acids in which each position identifies the amino acid (if a specific amino acid is identified) or a subset of amino acids (if a position is identified as variable) most likely to be found at a specific position in a 3-PG phosphatase. Many amino acids in SEQ ID NO: 7 are highly conserved and 3-PG phosphatases suitable for use in accordance with the methods of the present disclosure will comprise a substantial number, and sometimes all, of these highly conserved amino acids at positions aligning with the location of the indicated amino acid in SEQ ID NO: 7. In various embodiments, proteins suitable for use in accordance with the methods of the present disclosure have 3-PG phosphatase activity and comprise an amino acid sequence with at least 40%, at least 50%, at least 60%, or at least 70% sequence identity with SEQ ID NO: 7. For example, the Ashbya aceri yeast 4-nitrophenylphosphatase UniProt ID: R9XA12 sequence is 62% identical to consensus sequence #1 SEQ ID NO: 7, and is therefore encompassed by consensus sequence #1 SEQ ID NO: 7. The highly conserved amino acids in SEQ ID NO: 7 are D25, F27, F29, D30, C31, D32, G33, V34, W36, P43, E47, L52, K57, F61, V62, T63, N64, N65, K68, S69, R70, Y73, K76, F77, G81, F91, S93, A98, G118, G121, E125, L126, G130, G135, D161, V167, G170, L171, Y177, L187, F196, T199, N200, D202, T204, P206, G209, G214, A215, G216, G235, K236, P237, M241, 1245, M261, G263, D264, R265, T268, D269, F272, G273, L278, T281, V284, L285, and G287. Example 5 demonstrates how recombinant host cells comprising many different heterologous nucleic acids encoding 3-PG phosphatase enzymes homologous SEQ ID NO: 7 increase glyceric acid production as compared to parental host cells that did not comprise said genetic modifications.

In other embodiments, any protein that shares the specific function of 3-PG phosphatase as described herein can be used in a recombinant host cell of the disclosure despite comprising insufficient sequence identity (i.e., less than 40% identity) with the 3-PG phosphatase consensus sequence.

### 2.2.1.2 2-Phosphoglycerate Phosphatase

In addition to 3-PG phosphatase enzymes, other enzymes can be used to convert glycolytic intermediates into glycerate. In some embodiments, the recombinant host cells comprise a 2-PG phosphatase; in these embodiments, the 2-PG phosphatase (EC #3.1.3.20) catalyzes another step in the glyceric acid pathway, wherein one molecule of 2-PG and one molecule of water are converted to one molecule of glycerate and one molecule of orthophosphate (Table 1). In some embodiments where recombinant host cells comprise a 3-PG phosphatase, said recombinant host cells further comprise a 2-PG phosphatase.

Any enzyme is suitable for use in accordance with this disclosure so long as the enzyme is capable of catalyzing said 2-PG phosphatase reaction described above. In an embodiment of the present disclosure, a glyceric acid pathway comprising a 2-PG phosphatase is calculated to thermodynamically favor the conversion of glucose to glycerate. In certain embodiments, recombinant host cells comprise one or more heterologous nucleic acids encoding a 2-PG phosphatase, wherein said recombinant host cells are capable of producing glycerate. In various embodiments, recombinant host cells may comprise multiple copies of a single heterologous nucleic acid and/or multiple copies of two or more heterologous nucleic acids. Recombinant host cells comprising multiple heterologous nucleic acids may comprise any number of heterologous nucleic acids.

Although phosphatase enzymes are ubiquitous in nature, no enzyme is known to specifically catalyze this reaction. Similar to 3-PG phosphatase, the 2-PG phosphatase of the present disclosure may be derived from a variety of phosphatases. Some substrate-promiscuous phosphatases have been reported with activity towards 2-PG, but these enzymes also dephosphorylate a broad range of glycolytic intermediates and other phosphorylated compounds, and are either not specific enough or not active enough toward 2-PG. Further, not all phosphatases are substrate-promiscuous, and many substrate-specific phosphatases have also been characterized with good activity toward compounds structurally similar to 2-PG, including phosphoserine (EC #3.1.3.3), glycerol-3-phosphate (EC #3.1.3.21) and phosphoglycolate (EC #3.1.3.18). Thus, although any enzyme, particularly, any phosphoglycerate monoester hydrolase (EC #3.1.3.X), is suitable for use in accordance with this disclosure so long as the enzyme is capable of catalyzing said 2-PG phosphatase reaction, not any such natural enzyme is able to catalyze said reaction with sufficient specificity or efficiency to produce reasonable product yields and/or titers. In many embodiments, the 2-PG phosphatase has one or more active site mutations. In some embodiments, the one or more active site mutations lies in the active site, catalytic site, or substrate binding site.

Methods for identifying and improving enzymes towards desired 2-PG phosphatase activity are disclosed below in section 2.2.2. The methods described herein for protein mutagenesis, expression, purification and characterization are methods widely-practiced by practitioners skilled in the art, who will appreciate that a wide variety of commercial solutions are available for such endeavors. Briefly, methods that may be used (as described in section 2.2.2) comprise random and rational mutagenesis methods such as site-specific mutagenesis, error-prone PCR, and directed evolution. Libraries of mutated genes or strains are generated, and mutagenized proteins are characterized, and integrated into the genome of recombinant host cells for further strain characterization. The methods disclosed may be adapted as needed depending on the target enzyme properties desired.

In some embodiments, the 2-PG phosphatase enables improved pathway flux. In some embodiments, the 2-PG phosphatase enables increased yield, titer and/or productivity. In some embodiments, the 2-PG phosphatase enables increased substrate specificity. In some embodiments, the 2-PG phosphatase displays improved kinetic properties, such as Kₘ and k_{cat}. In some embodiments, the 2-PG phosphatase is a product of one or more protein engineering cycles. In some embodiments, the 2-PG phosphatase comprises one or more point mutations. In some embodiments, the 2-PG phosphatase has improved Kₘ and/or k_{cat} for the substrate 2-PG. In some embodiments, the 2-PG phosphatase has Kₘ≤3 mM with 2-PG as substrate. In some embodiments, the 2-PG phosphatase has k_{cat}≥10 per second with 2-PG as substrate. In some embodiments, the 2-PG phosphatase has an improved k_{cat}/Kₘ compared to the protein that it was derived from.

In some embodiments, the 3-PG phosphatase is derived from a phosphoserine phosphatase (EC #3.1.3.3), glycerol-1-phosphatase (EC #3.1.3.21), or phosphoglycolate phosphatase (EC #3.1.3.18).

In some embodiments, the 2-PG phosphatase is derived from a bacterial source. In many of these embodiments, the 2-PG phosphatase is derived from a cell belonging to a genus selected from the group comprising Escherichia, Mycobacterium, Haemophilus and Synechococcus. Non-limiting examples of bacterial enzymes comprise Escherichia coli UniProt ID: P32662, Mycobacterium avium UniProt ID: AOQJI1, Haemophilus somnus UniProt ID: Q011W8, Synechococcus elongatus UniProt ID: Q55320, and Escherichia coli UniProt ID: P27848.

In some embodiments, the 2-PG phosphatase is derived from the Escherichia coli phosphoglycolate phosphatase (abbv. EcGPH; UniProt ID: P32662; SEQ ID NO: 1). EcGPH catalyzes the conversion of one molecule of 2-phosphoglycolate (2P-Gly) and one molecule of water to one molecule of glycolate and one molecule of phosphate. 2P-Gly and 2-PG are structurally similar, making EcGPH a good candidate for mutagenesis. In various embodiments, the 2-PG phosphatase has one or more active site mutations.

In some embodiments, the 2-PG phosphatase is derived from the Mycobacterium avium phosphoserine phosphatase (abbv. MaSERB; UniProt ID: AOQJI1; SEQ ID NO: 2; PDB: AOQJI1). MaSERB catalyzes the conversion of one molecule of O-phospho-L(or D)-serine and one molecule of water to one molecule of L (or D)-serine and one molecule of phosphate. O-phospho-L(or D)-serine and 2-PG are structurally similar, making MaSERB a good candidate for mutagenesis. In various embodiments, the 3-PG phosphatase has one or more active site mutations comprising E196S, T, N, Q, A, V, I or L; D189S, T, N, Q, A, V, I or L; or any combination thereof.

In some embodiments, the 2-PG phosphatase is derived from the Haemophilus somnus phosphoglycolate phosphatase (abbv. HsGPH; UniProt ID: Q011W8; SEQ ID NO: 3; PDB: 2HSZ). HsGPH catalyzes the conversion of one molecule of 2P-Gly and one molecule of water to one molecule of glycolate and one molecule of phosphate. In various embodiments, the 3-PG phosphatase derived from HsGPH has one or more active site mutations at the amino acid positions comprising A49, Q178, N179, N199, Y200, N201, or any combination thereof.

In some embodiments, the 2-PG phosphatase is derived from the Synechococcus elongates alkaline phosphatase (abbv. SePHOV; UniProt ID: Q55320, SEQ ID NO: 6). SePHOV catalyzes the conversion of phosphate monoester and water to alcohol and phosphate. In various embodiments, the 2-PG phosphatase is without an N-terminal signal sequence. In some embodiments, the 2-PG phosphatase is without its first 20 amino acids. In various embodiments, the 2-PG phosphatase has one or more active site mutations.

In some embodiments, the 2-PG phosphatase is derived from the Escherichia coli phosphosugar phosphatase (abbv. EcYIGL; UniProt ID: P27848; SEQ ID NO: 8). EcYIGL catalyzes the conversion of one molecule of pyridoxal 5′-phosphate and one molecule of water to one molecule of pyridoxal and one molecule of phosphate. In various embodiments, the 2-PG phosphatase derived from EcYIGL has one or more active site mutations.

In some embodiments, the 2-PG phosphatase is derived from a eukaryotic source. In many of these embodiments, the 2-PG phosphatase is derived form a cell belonging to a genus selected from the group comprising Saccharomyces, Pichia, and Homo.

Non-limiting examples of eukaryotic enzymes comprise Saccharomyces cerevisiae UniProt ID: P40106, Saccharomyces cerevisiae UniProt ID: P19881, Saccharomyces cerevisiae UniProt ID: A6ZX98, Saccharomyces cerevisiae UniProt ID: G2WBT3, Saccharomyces cerevisiae UniProt ID: C7GJQ5, Saccharomyces cerevisiae UniProt ID: P36151, Pichia kudriavzevii UniProt ID: AOAIZ8JUP5, Pichia kudriavzevii UniProt ID: A0A099NZ38, Pichia kudriavzevii UniProt ID: A0A1Z8JN52, and Homo sapiens UniProt ID: A6NDG6.

In some embodiments, the 2-PG phosphatase is derived from the Saccharomyces cerevisiae glycerol-1-phosphate phosphatase (abbv. ScGPP2; UniProt ID: P40106; SEQ ID NO: 4). ScGPP2 catalyzes the conversion of one molecule of glycerol-1-phosphate and one molecule of water to one molecule of glycerol and one molecule of phosphate. In various embodiments, the 2-PG phosphatase derived from ScGPP2 has one or more active site mutations comprising N204R, H, K, Q, A or Y.

In some embodiments, the 2-PG phosphatase is derived from the Homo sapiens glycerol-3-phosphate phosphatase (abbv. HsPGP; UniProt ID: A6NDG6; SEQ ID NO: 5). HsPGP catalyzes the conversion of glycerol-3-phosphate and water to glycerol and phosphate. In various embodiments, the 2-PG phosphatase has one or more active site mutations.

In some embodiments, the 2-PG phosphatase is derived from Saccharomyces cerevisiae 4-nitrophenylphosphatase (abbv. ScPHO13; UniProt ID: P19881; SEQ ID NO: 9). ScPHO13 catalyzes the conversion of one molecule of 4-nitrophenyl phosphate and one molecule of water to one molecule of 4-nitrophenol and one molecule of phosphate. In some embodiments, the 2-PG phosphatase derived from ScPHO13 has one or more active site mutations.

In some embodiments, the 2-PG phosphatase is derived from Pichia kudriavzevii Pho13 protein (abbv. PkPHO13; UniProt ID: A0A1Z8JUP5; SEQ ID NO: 10). PKPHO13 catalyzes the conversion of one molecule of 4-nitrophenyl phosphate and one molecule of water to one molecule of 4-nitrophenol and one molecule of phosphate. In some embodiments, the 3-PG phosphatase derived from ScPHO13 has one or more active site mutations.

In some embodiments, the 2-PG phosphatase is derived from Pichia kudriavzevii ORF64 protein (abbv. PKORF64; UniProt ID: A0A099NZ38; SEQ ID NO: 11). In some embodiments, the 3-PG phosphatase derived from PKORF64 has one or more active site mutations.

In some embodiments, the 2-PG phosphatase is derived from Pichia kudriavzevii ORF423 protein (abbv. PkORF423; UniProt ID: A0A1Z8JN52; SEQ ID NO: 12). In some embodiments, the 3-PG phosphatase derived from PKORF423 has one or more active site mutations.

In some embodiments, the 2-PG phosphatase is derived from Saccharomyces cerevisiae uncharacterized protein YKR070W (abbv. ScYKR070W; UniProt ID: P36151; SEQ ID NO: 13). In some embodiments, the 2-PG phosphatase derived from PKORF423 has one or more active site mutations.

In some embodiments, the 2-PG phosphatase has a Km of less than about 5 mM with 2-PG as the substrate. In some embodiments, the 2-PG phosphatase has a Kₘ for 2-PG that is less than Kₘ for other glycolytic intermediates. In some embodiments, the 2-PG phosphatase has a k_{cat} of greater than about 10 turnovers per second with 2-PG as the substrate. In some embodiments, the 2-PG phosphatase has a k_{cat} for 2-PG that is greater than the k_{cat} for other glycolytic intermediates.

In many embodiments, recombinant host cells comprise one or more heterologous nucleic acids encoding a 2-PG phosphatase wherein said recombinant host cells are capable of producing glycerate. In various embodiments, proteins suitable for use in accordance with methods of the present disclosure have 2-PG phosphatase activity and comprise an amino acid sequence with at least 40%, at least 50%, at least 60%, at least 70%, at least 80%, at least 90%, or at least 95% sequence identity with SEQ ID NO: 1, SEQ ID NO: 2, SEQ ID NO: 3, SEQ ID NO: 4, SEQ ID NO: 5, SEQ ID NO: 6, SEQ ID NO: 8, SEQ ID NO: 9, SEQ ID NO: 10, SEQ ID NO: 11, SEQ ID NO: 12, or SEQ ID NO: 13. In many embodiments, the recombinant host cell is a P. kudriavzevii strain.

A consensus sequence is useful in identifying 2-PG phosphatases suitable for use in accordance with the methods of the present disclosure. A 2-PG phosphatase encompassed by a 2-PG phosphatase consensus sequence provided herein has an enzymatic activity that is identical, or essentially identical, or at least substantially similar with respect to ability to convert one molecule of 2-PG and one molecule of water to one molecule of glycerate and one molecule of orthophosphate. Thus, any protein substantially homologous to 2-PG phosphatase as described herein can be used in a recombinant host cell of the present disclosure.

Consensus sequence #1 (SEQ ID NO: 7) provides the sequence of amino acids in which each position identifies the amino acid (if a specific amino acid is identified) or a subset of amino acids (if a position is identified as variable) most likely to be found at a specific position in a 2-PG phosphatase. Many amino acids in SEQ ID NO: 7 are highly conserved and 2-PG phosphatases suitable for use in accordance with the methods of the present disclosure will comprise a substantial number, and sometimes all, of these highly conserved amino acids at positions aligning with the location of the indicated amino acid in SEQ ID NO: 7. In various embodiments, proteins suitable for use in accordance with the methods of the present disclosure have 2-PG phosphatase activity and comprise an amino acid sequence with at least 40%, at least 50%, at least 60%, or at least 70% sequence identity with SEQ ID NO: 7. For example, the Ashbya aceri yeast 4-nitrophenylphosphatase UniProt ID: R9XA12 sequence is 62% identical to consensus sequence #1 SEQ ID NO: 7, and is therefore encompassed by consensus sequence #1 SEQ ID NO: 7. The highly conserved amino acids in SEQ ID NO: 7 are D25, F27, F29, D30, C31, D32, G33, V34, W36, P43, E47, L52, K57, F61, V62, T63, N64, N65, K68, S69, R70, Y73, K76, F77, G81, F91, S93, A98, G118, G121, E125, L126, G130, G135, D161, V167, G170, L171, Y177, L187, F196, T199, N200, D202, T204, P206, G209, G214, A215, G216, G235, K236, P237, M241, 1245, M261, G263, D264, R265, T268, D269, F272, G273, L278, T281, V284, L285, and G287.

In other embodiments, any protein that shares the specific function of 2-PG phosphatase as described herein can be used in a recombinant host cell of the disclosure despite comprising insufficient sequence identity (i.e., less than 40% identity) with the 2-PG phosphatase consensus sequence.

2.2.2 Methods to Identify and/or Improve Enzymes in the Glyceric Acid Pathway

The present disclosure provides the construction and characterization of 3-PG phosphatases and 2-PG phosphatases in accordance with the present disclosure. The following methods have been developed for mutagenesis and diversification of genes for engineering specific or enhanced properties of targeted enzymes. The methods disclosed may be adapted as needed depending on the target enzyme properties desired. In some instances, the disclosed methods are suitable for use in engineering enzymes towards improved 3-PG phosphatase activity or 2-PG phosphatase activity of the glyceric acid pathway. In some embodiments, the 3-PG phosphatase is derived from an enzyme with native activity towards a substrate that is structurally similar to 3-PG. In many embodiments, the 3-PG phosphatase is derived from a phosphoric monoester hydrolase (EC #3.1.3.X), which comprises 3-PG phosphatase (EC #3.1.3.38), 2-PG phosphatase (EC #3.1.3.20), phosphoserine phosphatase (EC #3.1.3.3), glycerol-3-phosphate phosphatase (EC #3.1.3.21) and phosphoglycolate phosphatase (EC #3.1.3.18). Similarly, in embodiments that comprise a 2-PG phosphatase, the 2-PG phosphatase is derived from an enzyme with native activity towards a substrate that is structurally similar to 2-PG. In many embodiments, the 2-PG phosphatase is derived from a phosphoric monoester hydrolase (EC #3.1.3.X), which comprises 3-PG phosphatase (EC #3.1.3.38), 2-PG phosphatase (EC #3.1.3.20), phosphoserine phosphatase (EC #3.1.3.3), glycerol-3-phosphate phosphatase (EC #3.1.3.21) and phosphoglycolate phosphatase (EC #3.1.3.18).

Methods described herein for protein mutagenesis, identification, expression, purification, and characterization are methods widely-practiced by practitioners skilled in the art, who will appreciate that a wide variety of commercial solutions are available for such endeavors. Practitioners will understand that identification of mutated proteins comprise activity screens and phenotypic selections.

### 2.2.2.1 Generating Protein Libraries Via Mutagenesis

Enzymes that are identified as good mutagenesis starting points enter the protein engineering cycle, which comprises protein mutagenesis, protein identification, protein expression, protein characterization, recombinant host cell characterization, and any combination thereof. Iterative rounds of protein engineering are typically performed to produce an enzyme variant with properties that are different from the template/original protein. The enzyme variants of the present disclosure comprise 3-PG phosphatase and 2-PG phosphatase. Examples of enzyme characteristics that are improved and/or altered by protein engineering comprise, for example, substrate binding (Kₘ; i.e., a measure of enzyme binding affinity for a particular substrate) that includes non-natural substrate selectivity/specificity; enzymatic reaction rates (k_{cat}; the turnover rate of a particular enzyme-substrate complex into product and enzyme), to achieve desired pathway flux; temperature stability, for high temperature processing; pH stability, for processing in extreme pH ranges; substrate or product tolerance, to enable high product titers; removal of inhibition by products, substrates or intermediates; expression levels, to increase protein yields and overall pathway flux; oxygen stability, for operation of air sensitive enzymes under aerobic conditions; and anaerobic activity, for operation of an aerobic enzyme in the absence of oxygen. In some embodiments, the enzyme variant enables improved glyceric acid pathway flux. In some embodiments, the enzyme variant enables increased glycerate yield, titer and/or productivity. In some embodiments, the enzyme variant enables increased substrate specificity. In some embodiments, the enzyme variant displays improved kinetic properties, such as decreased Kₘ and/or increased k_{cat}. In some embodiments, the enzyme variant has increased Kₘ and/or decreased k_{cat} for the substrate 3-PG or 2-PG. In some embodiments, the enzyme variant has Kₘ≤3 mM with 3-PG or 2-PG as substrate. In some embodiments, the enzyme variant has k_{cat}≥10 turnovers per second with 3-PG or 2-PG as substrate. In some embodiments, the enzyme variant is a product of one or more protein engineering cycles. In some embodiments, the enzyme variant comprises one or more point mutations.

In general, random and rational mutagenesis approaches are acceptable methods for generating DNA libraries of mutant proteins. Error-prone PCR is a random mutagenesis method widely used for generating diversity in protein engineering, is not only fast and easy, but it is also a method that has successfully produced mutated enzymes with altered activity from a wild type DNA template. (Wilson, D. S. & Keefe, A. D. Random mutagenesis by PCR. Curr. Protoc. Mol. Biol. Chapter 8, Unit 8.3 (2001.) To help increase the odds of identifying an enzyme with 3-PG/2-PG phosphatase activity, rational mutagenesis of a small number of active site mutations is also useful. For example, in embodiments wherein the starting protein for 3-PG phosphatase engineering is the Escherichia coli (E. coli) phosphoglycolate (EcGPH), atomic structures of EcGPH homologs are available to guide structural modeling of EcGPH with 3-PG bound in the active site. Structural modeling allows one to identify amino acids in the active site involved with substrate recognition. Other mutagenesis approaches that could be used comprise DNA shuffling and combinatorial mutagenesis. In some embodiments, the mutagenesis step is carried out more than once, resulting in iterative rounds of engineering.

Following library generation, mutated genes are typically cloned for expression in a host organism and in many cases the proteins are subsequently purified for in vitro phosphatase screening. In some embodiments, the host organism is E. coli. Mutated genes are cloned in a suitable expression plasmid comprising an auto-inducible promoter upstream of the gene and a His-tag sequence downstream of the gene. The proteins expressed from such a plasmid are isolated from whole cell lysate with Ni-NTA affinity purification methods, such as the Takara Capturem His-tagged Purification 96. Purified proteins then enter an in vitro phosphatase screen for characterization. The Ni-NTA affinity purification step helps to reduce background noise in the in vitro phosphatase screen by removing both native phosphatases as well as any free orthophosphate present in the lysate.

### 2.2.2.2 Generating Strain Libraries Via Directed Evolution

In another aspect of this disclosure, directed evolution methods are used to identify enzymes with phosphatase activity and/or improved the kinetic parameters (for example, decreasing the enzyme Kₘ and/or increasing the enzyme k_{cat} when using 3-PG as the substrate) of enzymes exhibiting suboptimal activity toward 3-PG or 2-PG. Directed evolution approaches are useful in generating strain libraries with a wide diversity of mutations wherein the mutations are driven by the process of natural selection given the constraints provided to the organism in its growth environment. Evolution approaches provide an effective and impartial way of introducing sequence mutations that give rise to functional change at an organism scale, enabling practitioners to explore non-intuitive mutations in the universe of possibilities that lie beyond the confines of one's understanding about structure-function specificity.

In some embodiments, a screen is designed to monitor the progress of evolution over time. In some of these embodiments, it is useful to link desired mutagenesis with a measurable phenotype so that the rate of evolution can be monitored over an extended period of time. In some of these embodiments, the measurable phenotype comprises cell growth, glucose consumption, and metabolite production. In some embodiments, the directed evolution experiment is designed so that mutations acquired in the target gene(s) is a measurable phenotype that is advantageous to the organism. In some of these embodiments, the advantageous measurable phenotype comprises cellular fitness, energy production, growth rate, tolerance to toxicity, and tolerance to extreme culture conditions (such as high or low pH, high or low temperature, high or low osmotic pressure, drought, and nutrient limitation). In various embodiments, one or more synthetic metabolic pathways are constructed by introducing exogenous nucleic acids to recombinant host cells. In these embodiments, the one or more synthetic metabolic pathways provide a method of applying selective pressure or a method of selecting strain variants that result from directed evolution.

Besides a well-crafted selection assay before the evolution experiment begins, starting nucleic acid templates for proteins of interest (i.e., target gene(s) or parent gene(s)) are also identified. In embodiments of the present disclosure, enzymes that serve as a good starting point for 3-PG phosphatase or 2-PG phosphatase engineering are identified. In these embodiments, 3-PG phosphatase/2-PG phosphatase-encoding nucleic acids are integrated into the genome of recombinant host cells. In some embodiments, the 3-PG phosphatase is derived from an enzyme with native activity towards a substrate that is structurally similar to 3-PG. In some embodiments, the 3-PG phosphatase is derived from a phosphoric monoester hydrolase (EC #3.1.3.X). Similarly, in embodiments comprising a 2-PG phosphatase, the 2-PG phosphatase is derived from an enzyme with native activity towards a substrate that is structurally similar to 2-PG. In some embodiments, the 2-PG phosphatase is derived from a phosphoric monoester hydrolase (EC #3.1.3.X).

Once a selection is established and target genes (i.e., for 3-PG phosphatases/2-PG phosphatases, according to embodiments of the present disclosure) are identified and integrated into the genome of recombinant host cells, recombinant host cells enter the directed evolution cycle, wherein the directed evolution cycle comprises: (1) mutagenesis in response to selective pressure; (2) analysis of recombinant host cells in the generated library for measurable phenotypic differences that arise due to selective pressure; and (3) isolation and characterization of evolved variants.

In some embodiments, acquisition of a mutation in the target gene enables the recombinant host cell to overcome the selective pressure. In some embodiments, recombinant host cells are passaged throughout the course of mutagenesis with selective pressure. In various embodiments, the selective pressure comprises nutrient limitation, cellular toxicity, and extreme culture conditions that further comprise high or low pH, high or low temperature, and high or low osmotic pressure. In some embodiments, the recombinant host cells are initially propagated without selective pressure prior to mutagenesis.

After exposure to selective pressure for some period of time, the evolved or evolving strains are screened for a change in phenotype in response to selective pressure. Non-limiting examples of phenotypic change comprise faster glucose consumption, faster cell growth, higher flux through a metabolic pathway or pathways, improved product yield/titer/productivity, decreased byproduct yield/titer, increased tolerance to toxicity, or increased tolerance to extreme culture conditions.

### 2.2.2.3 Enzyme Characterization

Following protein library generation, protein variants can be screened for

phosphatase activity with any of a variety of published assays and commercial kits available for the measurement of free phosphate which can be used to screen 3-PG phosphatase/2-PG phosphatase activity. An example of a commercial in vitro assay is the Sigma-Aldrich assay kit catalog #MAK307 that produces a colorimetric output upon detection of free phosphate. With 3-PG as the substrate, for example, kinetic parameters Kₘ (i.e., binding affinity for a substrate) and k_{cat} (i.e., turnover rate of an enzyme-substrate complex into product and enzyme) are calculated from the assay. After identifying 3-PG phosphatases with this initial screen, the same assay kits can be used to test 3-PG phosphatase substrate specificity. In addition to 3-PG, a variety of glycolytic intermediates and other cellular metabolites can be provided as substrates in the assay to determine if the engineered 3-PG phosphatase can dephosphorylate other substrates, leading to byproducts that could be problematic in a commercial process. Non-limiting examples of intracellular metabolites that can serve as substrates comprise phosphoglycolate, glucose-6-phosphate, frustose-6-phosphate and glyceraldehyde-3-phosphate. 3-PG phosphatase Kₘ and k_{cat} are calculated for these substrates. In some embodiments, protein isolation is carried out prior to in vitro characterization, with commercially available kits such as the Takara Capturem His-tagged Purification 96.

Following strain library generation and screening, strain variants with desired evolved phenotype(s) are typically isolated and characterized. In some embodiments, mutations are acquired by the nucleic acids encoding target proteins. In these embodiments, nucleic acids encoding target proteins are sequenced so that acquired mutations are identified. In other embodiments, mutations are acquired by nucleic acids native to the recombinant host cells. In some embodiments, target proteins are analyzed for 3-PG phosphatase activity.

In some embodiments, iterative rounds of protein engineering are performed to produce enzyme variants with optimized properties, wherein the iterative rounds of protein engineering comprise rational mutagenesis, random mutagenesis, and directed evolution. In these embodiments, select variants from preceding rounds of protein engineering are identified for further protein engineering. Non-limiting examples of such properties comprise improved enzyme kinetics for specificity and/or turnover, improved pathway flux, increased metabolite yield, decreased byproduct yield. In some embodiments, culture medium or fermentation broth is analyzed for the presence of metabolites such as glyceric acid and/or byproducts, wherein the method of analysis is HPLC (high-performance liquid chromatography).

### 2.3 Glycolic Acid Pathway

Provided herein in certain embodiments are recombinant host cells having at least one active glycolic acid pathway from glycerate to glycolate. Recombinant host cells having an active glycolic acid pathway as used herein produce active enzymes useful to catalyze each metabolic reaction in a glycolic acid pathway, and therefore are capable of producing glycolic acid in measurable yields and/or titers when cultured under suitable conditions. Recombinant host cells having an active glycolic acid pathway comprise one or more heterologous nucleic acids encoding glycolic acid pathway enzymes and are capable of producing glycolate.

Recombinant host cells may employ combinations of metabolic reactions for biosynthetically producing the compounds of the present disclosure. The biosynthesized compounds produced by the recombinant host cells comprise glycolate, glycolic acid, and the intermediates, products and/or derivatives of the glycolic acid pathway. The biosynthesized compounds can be produced intracellularly and/or secreted into the fermentation medium. In certain embodiments, recombinant host cells of the present disclosure comprise a glycolic acid pathway that proceeds via glycerate.

### 2.3.1 Glycolic Acid Pathway Enzymes

The glycolic acid pathway described herein produces glycolate from glucose via the 3-PG phosphatase of the glyceric acid pathway with the following balanced, stoichiometric equation:

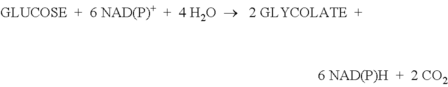

In a particular embodiment of the present disclosure, the glycolic acid pathway comprises three enzymes: hydroxypyruvate reductase (EC #1.1.1.81), hydroxypyruvate decarboxylase (EC #4.1.1.40), and glycoaldehyde reductase (EC #1.2.1.21). In some embodiments, all three glycolic acid pathway enzymes are required to convert glycerate to glycolate via the glycolic acid pathway of the present disclosure.

TABLE 2
Enzymes in the glycolic acid pathway

| EC # | Enzyme name | Reaction catalyzed |
|---|---|---|
| 1.1.1.81 | Hydroxypyruvate | Glycerate + oxidized cofactor → |
| | reductase | Hydroxypyruvate + reduced cofactor |
| 4.1.1.40 | Hydroxypyruvate | Hydroxypyruvate + H₂O → |
| | decarboxylase | Glycoaldehyde + CO₂ |
| 1.2.1.21 | Glycoaldehyde | Glycoaldehyde + oxidized cofactor → |
| | reductase | Glycolate + reduced cofactor |

The glycolic acid pathway of the present disclosure is calculated to thermodynamically favor the conversion of glucose to glycolate. The advantaged thermodynamics of the pathway will help to achieve high glycolic acid yields, titers and/or productivities. The conversion of glucose to glycolate using the glyceric acid and glycolic acid pathways of the present disclosure as described herein has a calculated ΔᵣG^{m} of −209.2+/−9.2 KJ/mol, indicative of a strong driving force that pushes the reaction to completion. Further, the pathway has net accumulation of 6 mol of NAD(P)H for every mol of glycolate produced from glucose, which can be used to help the cell generate ATP in aerobic fermentation, which is useful for product export, cellular maintenance, and other vital activities.

Specifically, two of the three reactions in the glycolic acid pathway are either thermodynamically irreversible (i.e., have a negative ΔᵣG^{m} as described above), or are catalyzed by unidirectional enzymes (i.e., where the enzyme mechanism does not permit the reverse reaction); enzymes catalyzing these reactions are referred to as irreversible enzymes. The irreversible glycolic acid pathway enzymes are hydroxypyruvate decarboxylase (in the second step) and glycoaldehyde reductase (in the last step). As the pathway intermediates pass through these steps, they become locked into the portion of the glycolic acid pathway downstream of each irreversible enzyme. Generally speaking, when multiple enzymes in a metabolic pathway are irreversible this helps to increase product yields, titers, and productivities. Because the last two glycolic acid pathway steps are irreversible, a strong driving force pushes carbon out of the pathway to make the glycolate final product.

In certain embodiments, recombinant host cells comprise one or more heterologous nucleic acids encoding one, two, or all three enzymes of the glycolic acid pathway, or any combination thereof, wherein the heterologous nucleic acids are expressed in sufficient amounts to produce glycolate. In various embodiments, recombinant host cells may comprise multiple copies of a single heterologous nucleic acid and/or multiple copies of two or more heterologous nucleic acids. Recombinant host cells comprising multiple heterologous nucleic acids may comprise any number of heterologous nucleic acids.

The biosynthesized compounds produced by the recombinant host cells comprise glycolic acid, and the products and intermediates of the glycolic acid pathway, namely hydroxypyruvate, glycoaldehyde and glycolate. These products and intermediates can be produced intracellularly and/or are secreted into the fermentation medium.

The present disclosure also provides consensus sequences useful in identifying and/or constructing glycolic acid pathway enzymes suitable for use in accordance with the methods of the present disclosure. For example, a hydroxypyruvate reductase encompassed by hydroxypyruvate reductase consensus sequence provided herein has an enzymatic activity that is identical, or essentially identical, or at least substantially similar with respect to ability to convert one molecule of glycerate and one molecule of NAD(P)⁺ to one molecule of hydroxypyruvate and one molecule of NAD(P)H.

### 2.3.1.1 Hydroxypyruvate Reductase

Hydroxypyruvate reductase (EC #1.1.1.81) described herein catalyzes the conversion of one molecule of glycerate and one molecule of NAD(P)⁺ to one molecule of hydroxypyruvate and one molecule of NAD(P)H. Any enzyme is suitable for use in accordance with this disclosure so long as the enzyme is capable of catalyzing said hydroxypyruvate reductase reaction.

In some embodiments, the hydroxypyruvate reductase is derived from a bacterial source. In some embodiments, the hydroxypyruvate reductase is derived from a cell belonging to a genus selected from the group comprising Delftia, Hyphomicrobiium, Methylobacterium, Paracoccus, and Pyrococcus. Non-limiting examples of comprise Pyrococcus furiosus UniProt ID: Q8U3Y2, and Pyrococcus yayanosii UniProt ID: F8AEA4.

In some embodiments, the hydroxypyruvate reductase is derived from a eukaryotic source. In many embodiments, the hydroxypyruvate reductase is derived from a cell belonging to a genus selected from the group comprising Arabidopsis, Bos, Brassica, Chlorella, Citrullus, Cucumis, Cucurbita, Homo, Hordeum, Mus, Nicotiana, Pisum, Rattus, Spinacia, Sus, and Zea. Non-limiting examples comprise Arabidopsis thaliana UniProt ID: Q9C9W5, Arabidopsis thaliana UniProt ID: Q9LE33, Arabidopsis thaliana UniProt ID: Q9CA90, Arabidopsis thaliana UniProt ID: A0A119LPQ6, and Homo sapiens UniProt ID: Q9UBQ7.

In many embodiments, recombinant host cells comprise one or more heterologous nucleic acids encoding a hydroxypyruvate reductase wherein said recombinant host cells are capable of producing hydroxypyruvate. In various embodiments, proteins suitable for use in accordance with methods of the present disclosure have hydroxypyruvate reductase activity and comprise an amino acid sequence with at least 40%, at least 50%, at least 60%, at least 70%, at least 80%, at least 90%, or at least 95% sequence identity with UniProt ID: Q8U3Y2, UniProt ID: F8AEA4, UniProt ID: Q9C9W5, UniProt ID: Q9LE33, UniProt ID: Q9CA90, UniProt ID: A0A119LPQ6, and UniProt ID: Q9UBQ7. In many embodiments, the recombinant host cell is a P. kudriavzevii strain.

In other embodiments, any protein that shares the specific function of hydroxypyruvate reductase as described herein can be used in a recombinant host cell of the disclosure despite comprising insufficient sequence identity (i.e., less than 40% identity) with UniProt ID: Q8U3Y2, UniProt ID: F8AEA4, UniProt ID: Q9C9W5, UniProt ID: Q9LE33, UniProt ID: Q9CA90, UniProt ID: A0A119LPQ6, or UniProt ID: Q9UBQ7.

### 2.3.1.2 Hydroxypyruvate Decarboxylase

Hydroxypyruvate decarboxylase (EC #4.1.1.40) described herein catalyzes the conversion of one molecule of hydroxypyruvate and one molecule of water to one molecule of glycoaldehyde and one molecule of carbon dioxide. Any enzyme is suitable for use in accordance with this disclosure so long as the enzyme is capable of catalyzing said hydroxypyruvate decarboxylase reaction.

In some embodiments, the hydroxypyruvate decarboxylase is derived from a eukaryotic source. In many embodiments, the hydroxypyruvate decarboxylase is derived from a cell belonging to a genus selected from the group comprising Bos, Canis, Oryctolagus, Rattus and Sus.

### 2.3.1.3 Glycoaldehyde Reductase

Glycoaldehyde reductase (EC #1.2.1.21) described herein catalyzes the conversion of one molecule of glycoaldehyde and one molecule of NAD (P) to one molecule of glycolate and one molecule of NAD (P) H. Any enzyme is suitable for use in accordance with this disclosure so long as the enzyme is capable of catalyzing said glycoaldehyde reductase reaction.

In many embodiments, the glycoaldehyde reductase is derived from a bacterial source. In many embodiments, the glycoaldehyde reductase is derived from a cell belonging to a genus selected from the group comprising Agrobacterium, Alcaligenes, Bacillus, Corynebacterium, Enterobacter, Escherichia, Flavobacterium, Klebsiella, Micrococcus, Pimelobacter, Protaminobacter, Proteus, Pseudomonas, Rhodococcus, Salmonella, and Staphylococcus.

In some embodiments, the glycoaldehyde reductase is the Escherichia coli ALDA protein (abbr. EcALDA; UniProt ID: P25553; SEQ ID NO: 30). In many embodiments, recombinant host cells comprise one or more heterologous nucleic acids encoding a glycoaldehyde reductase wherein said recombinant host cells are capable of producing glycolate. In various embodiments, proteins suitable for use in accordance with methods of the present disclosure have glycoaldehyde reductase activity and comprise an amino acid sequence with at least 40%, at least 50%, at least 60%, at least 70%, at least 80%, at least 90%, or at least 95% sequence identity with SEQ ID NO: 30. In many embodiments, the recombinant host cell is a P. kudriavzevii strain.

In other embodiments, any protein that shares the specific function of glycoaldehyde reductase as described herein can be used in a recombinant host cell of the disclosure despite comprising insufficient sequence identity (i.e., less than 40% identity) with SEQ ID NO: 30.

### 2.4 Ancillary Proteins

In addition to the glyceric acid pathway enzymes and/or downstream product pathway enzymes, ancillary proteins are other proteins that are overexpressed in recombinant host cells of the present disclosure whose overexpression results in an increase in glyceric acid and/or downstream product yields, productivities, and/or titers as compared to control, or cells that do not overexpress said proteins. Ancillary proteins function outside the glyceric acid pathway and/or the downstream product pathway, wherein each ancillary protein may play a role that indirectly boosts the recombinant host cell's ability to produce glyceric acid and/or downstream product. Ancillary proteins comprise any protein (excluding glyceric acid pathway enzymes and downstream product pathway enzymes) of any structure or function that can increase glyceric acid and/or downstream product yields, titers, or productivities when overexpressed. Non-limiting examples of classes of proteins comprise transcription factors, transporters, scaffold proteins, proteins that decrease byproduct accumulation, and proteins that regenerate or synthesize redox cofactors.

Provided herein in certain embodiments are recombinant host cells comprising one or more heterologous nucleic acids encoding one or more ancillary proteins wherein said recombinant host cell is capable of producing higher glyceric acid and/or downstream product yields, titers, or productivities as compared to control cells, or host cells that do not comprise said heterologous nucleic acid(s). In some embodiments, that host recombinant cell naturally produces glyceric acid and/or downstream product, and in these cases, the glyceric acid and/or downstream product yields, titers, and/or productivities are increased. In other embodiments, the recombinant host cell comprises one or more heterologous nucleic acids encoding one or more glycolic acid pathway enzymes and/or downstream product pathway enzymes.

In certain embodiments of the present disclosure, the recombinant host cells comprise one or more heterologous nucleic acids encoding one or more glyceric acid pathway enzymes and/or one or more downstream product pathway enzymes, and one or more heterologous nucleic acids encoding one or more ancillary proteins. In certain of these embodiments, the recombinant host cells may be engineered to express more of these ancillary proteins. In these particular embodiments, the ancillary proteins are expressed at a higher level (i.e., produced at a higher amount as compared to cells that do not express said ancillary proteins) and may be operatively linked to one or more exogenous promoters or other regulatory elements.

In certain embodiments, recombinant host cells comprise both endogenous and heterologous nucleic acids encoding one or more glyceric acid pathway enzymes and/or one or more downstream product pathway enzymes, and one or more ancillary proteins. In certain embodiments, the recombinant host cells comprise one or more heterologous nucleic acids encoding one or more glyceric acid pathway enzymes and/or one or more ancillary proteins, and one or more endogenous nucleic acids encoding one or more glyceric acid pathway enzymes and/or one or more downstream product pathway enzymes, and/or one or more ancillary proteins.

In certain embodiments, endogenous nucleic acids of ancillary proteins are modified in situ (i.e., on chromosome in the recombinant host cell genome) to alter levels of expression, activity, or specificity. In some embodiments, heterologous nucleic acids are inserted into endogenous nucleic acids of ancillary proteins.

### 2.4.1 Ancillary Proteins for Redox Cofactor Recycling

One type of ancillary proteins is proteins that recycle the redox cofactors that are produced during glycolic acid pathway activity. Redox balance is fundamental to sustained metabolism and cellular growth in living organisms. Intracellular redox potential is determined by redox cofactors that facilitate the transfer of electrons from one molecule to another within a cell. Redox cofactors in yeast comprise the nicotinamide adenine dinucleotides, NAD and NADP, the flavin nucleotides, FAD and FMN, and iron sulfur clusters (Fe—S clusters).

Redox constraints play a role in end-product formation. Additional reducing power is provided to produce compounds whose degree of reduction is higher than that of the substrate. Conversely, producing compounds with a degree of reduction lower than that of the substrate will force the synthesis of byproducts with higher degrees of reduction to compensate for excess reducing power generated from substrate oxidation. Thus, redox neutrality is maintained to ensure high end-product yields. For example, the glycolic acid pathway results in a net formation of 3 mol of NAD(P)H for each mol of glycolic acid in the cytosol. Re-oxidation of NAD(P)H to NAD(P)⁺ is involved in maintaining the thermodynamic driving force necessary for efficient and rapid glycolic acid production. This means that other processes in the cell can operate to restore the redox imbalance caused. In yeast, overflow metabolism can occur to reoxidize surplus cytosolic NAD(P)H, leading to byproduct accumulation, thereby decreasing product titers, yields and/or productivities. Non-limiting examples of ancillary proteins that can be overexpressed to restore this redox balance comprise NADH dehydrogenase and NADH oxidase. The enzymes of the glycolic acid pathway function in the cytosol, thus creating a redox imbalance with an excess of NAD(P)H in the cytosol. In certain embodiments, the ancillary proteins are expressed in the cytosol of recombinant host cells. In certain embodiments, the ancillary proteins are associated with the mitochondrial or cell membrane of the recombinant host cells.

In some embodiments, recombinant host cells comprise one or more heterologous and/or endogenous nucleic acids encoding a NADH dehydrogenase ancillary protein. The yeast NADH dehydrogenase catalyzes the oxidation of NAD(P)H to NAD(P)⁺, providing cytosolic NAD(P)H to the mitochondrial respiratory chain. Any NAD(P)H dehydrogenase can be used in accordance with the present disclosure so long as it is capable of oxidizing NAD(P)H to NAD(P)⁺ in the cytosol, restoring redox balance in the correct cellular space. In many embodiments, the NADH dehydrogenase ancillary protein is expressed in the cytosol. In some embodiments, the NADH dehydrogenase has an active site that is accessible to the cytosol. In some embodiments, the NADH dehydrogenase is a mitochondrial external NADH dehydrogenase. In yeast, the mitochondrial external NADH dehydrogenase is an inner-membrane mitochondrial protein with its catalytic site facing the intermembrane space. Because the mitochondrial outer membrane is permeable to most small molecules, the mitochondrial intermembrane space is considered to have the same environment as the cytosol. Therefore, the mitochondrial external NADH dehydrogenase contributes to the oxidation of cytosolic NAD(P)H and is a suitable ancillary protein for glycolic acid production. In some embodiments, the mitochondrial external NADH dehydrogenase is the P. kudriavzevii Nde1 protein (abbv. PKNDE1; SEQ ID NO: 20). In some embodiments, recombinant host cells comprise one or more heterologous nucleic acids encoding one or more proteins suitable for use in accordance with methods of the present disclosure having NAD(P)H dehydrogenase activity, and further comprise an amino acid sequence with at least 40%, at least 50%, at least 60%, at least 70%, at least 80%, at least 90%, or at least 95% sequence identity with SEQ ID NO: 20.

In some embodiments, recombinant host cells comprise one or more heterologous and/or endogenous nucleic acids encoding a NADH oxidase ancillary protein. The NADH oxidase converts NAD(P)H to NAD(P)⁺ and can restore redox balance in the recombinant host cell cytosol while reducing molecular oxygen, resulting in the formation of one mol water per mol NADH. Any NAD(P)H oxidase can be used in accordance with the present disclosure so long as it is capable of oxidizing NAD(P)H to NAD(P)⁺ and reducing molecular oxygen to water in the cytosol, restoring redox balance in the correct cellular space. In some embodiments, the NADH oxidase is the water-forming NADH oxidase. In some embodiments, the water-forming NADH oxidase is the Lactococcus lactis NoxE protein (abbv. LINOXE; UniProt ID A2RIB7; SEQ ID: NO 21). In some embodiments, the NADH oxidase is derived from a bacterial source. In some embodiments, the NADH oxidase is derived from an archaeal source. In some embodiments, recombinant host cells comprise one or more heterologous nucleic acids encoding one or more proteins suitable for use in accordance with methods of the present disclosure have NAD (P) H oxidase activity and comprise an amino acid sequence with at least 40%, at least 50%, at least 60%, at least 70%, at least 80%, at least 90%, or at least 95% sequence identity with SEQ ID NO: 21.

### 2.4.2 Ancillary Proteins for Redox Cofactor Biogenesis

As explained in preceding paragraphs, redox balance is crucial for cell growth and sustained metabolism. Two out of the four glycolic acid pathway enzymes utilize redox cofactors that can be generated, in addition to being recycled, for robust metabolism and cell vitality. In some embodiments of the present disclosure, recombinant host cells comprise a hydroxypyruvate reductase that that utilizes NAD (P). In some embodiments, recombinant host cells comprise a glycoaldehyde dehydrogenase that utilizes NAD(P)⁺. Thus, biogenesis and homeostasis of these cofactors are crucial for efficient catalysis of these enzymatic reactions.

The NAD and NADP cofactors are involved in electron transfer and contribute to approximately 12% of all biochemical reactions in a cell (Osterman A., EcoSal Plus, 2009). NAD is assembled from L-aspartate, dihydroxyacetone phosphate (DHAP; glycerone), phosphoribosyl pyrophosphate (PRPP) and ATP. The NADP is assembled in the same manner and further phosphorylated. In some embodiments, recombinant host cells comprise heterologous and/or endogenous nucleic acids encoding one or more ancillary proteins that facilitate NAD and NADP cofactor assembly. In some embodiments, the ancillary proteins comprise one, more or all proteins suitable for use in accordance with methods of the present disclosure having NAD and/or NADP assembly capability, NAD and/or NADP transfer capability, NAD and/or NADP chaperone capability, or any combination thereof.

Similarly, Fe—S clusters facilitate various enzyme activities that require electron transfer. Because both iron and sulfur atoms are highly reactive and toxic to cells, Fe-S cluster assembly requires carefully coordinated synthetic pathways in living cells. The three known pathways are the Isc (iron sulfur cluster) system, the Suf (sulfur formation) system, and the Nif (nitrogen fixation) system. Each of these systems has a unique physiological role, yet several functional components are shared between them. First, a cysteine desulfurase enzyme liberates sulfur atoms from free cysteine. Then, a scaffold protein receives the liberated sulfur for Fe—S cluster assembly. Finally, the Fe—S cluster is transferred to a target apoprotein. In some embodiments of the present disclosure, recombinant host cells comprise heterologous and/or endogenous nucleic acids encoding one or more ancillary proteins that facilitate Fe-S cluster assembly. In some embodiments, the ancillary proteins comprise one, more or all proteins of the Isc system, the Suf system, the Nif system, or any combination thereof. In some embodiments, recombinant host cells comprise one or more heterologous nucleic acids encoding one or more proteins suitable for use in accordance with methods of the present disclosure having cysteine desulfurase activity, Fe—S cluster assembly capability, Fe-S cluster transfer capability, iron chaperone capability, or any combination thereof.

2.4.3 Ancillary Proteins for Glyceric Acid and/or Downstream Product Transport

Another class of ancillary proteins useful for increasing glyceric acid and/or downstream product yields, titers, and/or productivities are organic acid transporter proteins. In some embodiments, recombinant host cells comprise one or more heterologous and/or endogenous nucleic acids encoding one or more organic acid transporter proteins. In many embodiments, the organic acid transporter is derived from a fungal source. In some embodiments, the organic acid transporter is selected from the group comprising Saccharomyces cerevisiae PDR12 (abbv. ScPDR12; UniProt ID: Q02785; SEQ ID NO: 22), Saccharomyces cerevisiae WAR1 (abbv. ScWAR1; UniProt ID: Q03631; SEQ ID NO: 23), Schizosaccharomyces pombe MAE1 (abbv. SpMAE1; UniProt ID: P50537; SEQ ID NO: 27), and Kluyveromyces marxianus PDC12 (abbv. KmPDR12; UniProt ID: WOT9C6; SEQ ID NO: 24). In some embodiments, recombinant host cells comprise one or more heterologous nucleic acids encoding one or more proteins suitable for use in accordance with methods of the present disclosure have glyceric acid and/or downstream product transporter activity. In some embodiments, recombinant host cells comprise one or more heterologous nucleic acids encoding one or more proteins that comprise an amino acid sequence with at least 40%, at least 50%, at least 60%, at least 70%, at least 80%, at least 90%, or at least 95% sequence identity with ScPDR12, ScWAR1, SpMAE1, and/or KmPDR12.

### 2.5 Decreasing or Eliminating Expression of Byproduct Pathway Enzymes

In an additional aspect of this disclosure, nucleic acids encoding byproduct pathway enzymes are disrupted in recombinant host cells of the present disclosure to increase glyceric acid and/or downstream product yields, productivities, and/or titers; and/or to decrease byproduct titers and/or yields as compared to control cells, or host cells that express native/undisrupted levels of said byproduct pathway enzymes. Byproduct pathway enzymes comprise any protein (excluding glyceric acid pathway enzymes and/or downstream product pathway enzymes) of any structure or function that can decrease glyceric acid and/or downstream product yields, titers, and/or productivities when disrupted because they utilize intermediates or products of the glyceric acid pathway and/or the downstream product pathway.

Byproducts that accumulate during glyceric acid and/or downstream product production can lead to: (1) lower glyceric acid and/or downstream product acid titers, productivities, and/or yields; and/or (2) accumulation of byproducts in the fermentation broth that increase the difficulty of downstream purification processes. In some embodiments, recombinant host cells may comprise genetic disruptions that encompass alternations, deletions, knockouts, substitutions, promoter modifications, premature stop codons, or knock-downs that decrease byproduct accumulation. In some embodiments, recombinant host cells comprising a disruption of one or more genes encoding a byproduct pathway enzyme will have altered performance characteristics as compared to cells without said genetic disruption(s), such as decreased or eliminated byproduct pathway enzyme expression, decreased or eliminated byproduct accumulation, improved glyceric acid and/or downstream product pathway activity, altered metabolite flux through the glyceric acid and/or downstream product pathway, higher glyceric acid and/or downstream product titers, glyceric acid and/or downstream product productivities, glyceric acid and/or downstream product yields, and/or altered cellular fitness.

One reason to decrease byproduct formation is to increase glyceric acid and/or downstream product pathway activity, resulting in an increased amount of glyceric acid and/or downstream product produced. In many embodiments, recombinant host cells of the present disclosure comprising one or more genetic disruptions of one or more genes encoding a byproduct pathway enzyme produce an increased glyceric acid and/or downstream product titer as compared to host cells that do not comprise said genetic disruption(s). In some of these embodiments, the glyceric acid titer in the fermentation broth is increased by 0.5 g/l, 1 g/l, 2.5 g/1, 5 g/1, 7.5 g/1, 10 g/l, or more than 10 g/l. In some of these embodiments, the downstream product titer in the fermentation broth is increased by 0.5 g/l, 1 g/1, 2.5 g/l, 5 g/1, 7.5 g/1, 10 g/l, or more than 10 g/l.

In addition to increasing glyceric acid and/or downstream product titers, decreasing byproduct formation can also help increase glyceric acid and/or downstream product yields. Because yield is independent of the volume of the fermentation broth, which can change during the course of a fermentation, it is often advantageous to measure glyceric acid and/or downstream product yields. In many embodiments, recombinant host cells of the present disclosure comprising one or more genetic disruptions of one or more genes encoding byproduct pathway enzymes produce an increased glyceric acid and/or downstream product yield as compared to host cells that do not comprise said genetic disruption. In some of these embodiments, the glyceric acid and/or downstream product yield is increased by 0.5%, 1%, 2.5%, 5%, 7.5%, 10%, or more than 10% (g-glyceric acid/g-substrate, or g-downstream product/g-substrate). The substrate in this yield calculation is the fermentation substrate, which is typically glucose, but may also be other, non-glucose substrates (for example, sucrose or glycerol).

Increasing glyceric acid and/or downstream product production can decrease manufacturing costs, and it can also be useful to disrupt genes encoding byproduct pathway enzymes in order to decrease byproduct formation. Byproducts are typically unwanted chemicals, are disposed of as waste, and their disposal can involve elaborate processing steps and containment requirements. Therefore, decreasing byproduct formation can lower production costs. In many embodiments, recombinant host cells of the present disclosure comprising one or more genetic disruptions of one or more genes encoding a byproduct pathway enzyme produces a lower byproduct titer as compared to host cells that do not comprise said genetic disruption. In some of these embodiments, a recombinant host cell of the disclosure comprising genetic disruption of one or more byproduct pathway enzymes produces a byproduct titer that is 0.5 g/l, 1 g/1, 2.5 g/1, 5 g/l, 7.5 g/1, 10 g/l, or greater than 10 g/l less than host cells that do not comprise said genetic disruption.

In many embodiments, recombinant host cells of the present disclosure comprising one or more genetic disruptions of one or more genes encoding a byproduct pathway enzyme produces a lower byproduct yield as compared to host cells that do not comprise said genetic disruption(s). In some of these embodiments, recombinant host cells comprise genetic disruption of one or more genes encoding byproduct pathway enzymes produce a byproduct yield that is 0.5%, 1%, 2.5%, 5%, 7.5%, 10%, or greater than 10% (percentage of g-byproduct for every g-substrate that is consumed) less than host cells that do not comprise said genetic disruption. As with the glycolic acid yield calculation, the substrate used in the byproduct yield calculation is the carbon source provided to the fermentation; this is typically glucose, sucrose, or glycerol, but may be any carbon substrate.

Non-limiting examples of byproducts that arise due to consumption of a glyceric acid pathway or a downstream product pathway substrate, intermediate or product comprise 2-phosphoglycerate, 3-phosphoglycerate, glycerol 3-phosphate, pyruvate, hydroxypyruvate, tartronate semialdehyde, 3-phosphonooxypyruvate, glyceraldehyde, 1-deoxy-D-xylulose 5-phosphate, DHAP, methylglyoxal, fructose 6-phosphoric acid, inositol-3-monophosphate, and 6-phospho-glucono-1,5-lactone, acetaldehyde, carbon dioxide, acetic acid, and ethanol. In the event of a redox imbalance, an undesirable excess of reduced or oxidized cofactors may also accumulate; thus, the redox cofactors NADH, NAD⁺, NADPH and NADP⁺ can also be considered byproducts.

A non-limiting list of enzyme-catalyzed reactions that utilize the glycolic acid pathway substrate (i.e., 3-PG or 2-PG), a glycolic acid pathway intermediate, or glycolic acid itself, are found in Table 3. Decreasing or eliminating expression of one, some or all of the genes encoding the enzymes in Table 3 can increase glyceric and/or glycolic acid production and/or decrease byproduct production. In many cases, the product of the enzyme-catalyzed reactions provided in Table 3 can accumulate in the fermentation broth; in such cases, this indicates that expression of the native gene encoding the listed enzyme should be reduced or eliminated. For example, the occurrence of dihydroxyacetone (abbv. DHAP; also known as glycerone) in the fermentation broth indicates that expression of a native gene encoding glycerol dehydrogenase should be decreased or eliminated. In some cases, the product of the specific reaction listed in Table 2 is further converted, either spontaneously or through the action of other enzymes, into a byproduct that accumulates in the fermentation broth. For example, dihydroxyacetone is generally metabolized to glycerol, which is found to accumulate in the fermentation broth. In cases where byproduct accumulation is due to the activity of multiple enzymes, one or more of the genes encoding the one or more byproduct pathway enzymes can be deleted or disrupted to reduce byproduct formation.

In some embodiments of the present disclosure, recombinant host cells comprise microbial strains with decreased or eliminated expression of one, some or all of the genes encoding enzymes listed in Table 3. In some embodiments, recombinant host cells comprise microbial strains with decreased byproduct accumulation wherein the byproducts are formed through the activity of one, some or all of the enzymes listed in Table 3. In some embodiments, recombinant host cells comprise microbial strains with decreased expression of pyruvate-utilizing enzymes. In some embodiments, recombinant host cells comprise microbial strains with decreased expression of glycolic acid-utilizing enzymes. In some embodiments, recombinant host cells comprise microbial strains with inability to catabolize or breakdown glyceric acid and/or glycolic acid. In some embodiments, recombinant host cells comprise genetic modifications that reduce the ability of the host cells to catabolize the glyceric acid and/or glycolic acid pathway intermediates except via the glyceric acid and/or glycolic acid pathway. In some embodiments, recombinant host cells comprise genetic modifications that decrease the ability of the host cells to catabolize pyruvate except via the glyceric acid and/or glycolic acid pathway.

TABLE 3
Enzyme-catalyzed reactions that consume glyceric
acid pathway substrate, intermediate or product

| Substrate | EC # | Enzyme name | Reaction formula |
|---|---|---|---|
| 3-Phospho- | 5.4.2.12 | Phosphoglycerate | 3-Phosphoglycerate → |
| glycerate | | mutase | 2-Phosphoglycerate |
| 3-Phospho- | 1.1.1.95 | Phosphoglycerate | 3-Phosphoglycerate + |
| glycerate | | dehydrogenase | Oxidized |
| | | | cofactor → 3- |
| | | | Phosphonooxypyruvate + |
| | | | Reduced |
| | | | cofactor |
| 2-Phospho- | 4.2.1.11 | Enolase | 2-Phosphoglycerate → |
| glycerate | | | Phosphoenolpyruvate + |
| | | | H₂O |
| Glycerate | 2.7.1.31 | Glycerate 3- | Glycerate + ATP → 3- |
| | | kinase | Phosphoglycerate + ADP |
| Glycerate | 2.7.1.165 | Glycerate 2- | Glycerate + ATP → 2- |
| | | kinase | Phosphoglycerate + ADP |
| Glycerate | 1.1.1.60 | Tartronate | Glycerate + Oxidized |
| | | semialdehyde | cofactor → |
| | | reductase | Tartronate |
| | | | semialdehyde + |
| | | | Reduced cofactor |
| Hydroxy- | 2.6.1.45 | Serine- | Glyoxylate + |
| pyruvate | | glyoxylate | L-Serine → Glycine + |
| | | amino | Hydroxypyruvate |
| | | transferase | |
| Hydroxy- | 1.4.1.7 | Serine | NH₃ + |
| pyruvate | | dehydrogenase | Hydroxypyruvate + |
| | | | Reduced |
| | | | cofactor → Serine + |
| | | | H₂O + |
| | | | Oxidized cofactor |
| Hydroxy- | 1.1.1.2 | Alcohol | Hydroxyacetaldehyde + |
| acetaldehyde | | dehydrogenase | Reduced |
| | | | cofactor → |
| | | | Ethylene glycol + |
| | | | Oxidized cofactor |
| Glucose | 5.5.1.4 | Inositol 1- | Glucose 6- |
| 6-phosphate | | phosphate | phosphate → Inositol-3- |
| | | synthase | monophosphate |
| Glucose | 1.1.1.49 | Glucose-6- | Glucose 6- |
| 6-phosphate | | phosphate | phosphate + Oxidized |
| | | dehydrogenase | cofactor → 6- |
| | | | Phosphoglucono-1,5- |
| | | | lactone + Reduced |
| | | | cofactor |
| Fructose-1,6- | 3.1.3.11 | Fructose-1,6- | Fructose-1,6- |
| biphosphate | | biphosphate | bisphosphate + H₂O → |
| | | phosphatase | Fructose |
| | | | 6-phosphoric acid + |
| | | | Orthophosphate |
| DHAP | 1.1.1.8 | Glycerol-3- | DHAP + NAD(P)H → |
| | | phosphate | NAD(P)⁺ + |
| | | dehydrogenase | Glycerol 3-phosphate |
| DHAP | 3.1.3.X | Dihydroxyacetone | DHAP + H₂O → |
| | | phosphate | Dihydroxyacetone + |
| | | phosphatase | Phosphate |
| DHAP | 4.2.3.3 | Methylglyoxal | DHAP → |
| | | synthase | Orthophosphate + |
| | | | Methylglyoxal |

### 2.5.1 Decreasing or Eliminating Expression of Phosphoglycerate Mutase

In some embodiments, it is beneficial to decrease or eliminate carbon flux through glycolysis (downstream of 3-PG) by decreasing or eliminating expression of phosphoglycerate mutase (EC #5.4.2.12). Phosphoglycerate mutase catalyzes the conversion of one molecule of 3-PG to one molecule of 2-phosphoglycerate (2-PG) diverting 3-PG from the glyceric acid and/or downstream product pathway to glycolysis. In some cases, it is beneficial to eliminate expression of genes encoding phosphoglycerate mutase activity, and in some of these cases, it is necessary to grow the biomass on an alternative carbon source to glucose (for example, ethanol, acetate, pyruvate, or citrate). In other cases, it is beneficial to decrease expression of phosphoglycerate mutase such that a small amount of glucose is converted to pyruvate during the production phase of the fermentation.

Several amino acids in phosphoglycerate mutase homologs are highly conserved. Any protein that is a phosphoglycerate mutase homolog will comprise a substantial number of highly conserved amino acids. In P. kudriavzevii, the phosphoglycerate mutase is PkGPM1 (UniProt ID: A0A099P7A2; SEQ ID NO: 14). Decreasing or eliminating expression of one or more homologs of PkGPM1 is useful for increasing glycolic acid production and/or decreasing carbon flux through glycolysis. As described above, homologous proteins share substantial sequence identity with each other. Any protein that is homologous to the phosphoglycerate mutase of the present disclosure (UniProt ID: A0A099P7A2; SEQ ID NO: 14) will share substantial sequence identity.

In some embodiments, recombinant host cells comprise genetic disruptions in one or more phosphoglycerate mutase homologs. As defined above, genetic disruptions encompass nucleic acid deletions, knockouts, nucleic acid insertions, nucleic acid substitutions, nucleic acid mutations, premature stop codons and promoter modifications. In some embodiments, recombinant host cells of the present disclosure comprise a genetic disruption of a homologous phosphoglycerate mutase gene with at least 40%, at least 50%, at least 60%, at least 70%, at least 80%, at least 90%, at least 95%, or more than 95% homology when compared to PkGPM1 (UniProt ID: A0A099P7A2; SEQ ID NO: 14). In some of these embodiments, the recombinant host cell is a P. kudriavzevii strain. In some embodiments, recombinant host cells comprise one or more gene disruptions that produce altered, decreased or eliminated activity in one, two or all three, phosphoglycerate mutase proteins. In some of these other embodiments, the recombinant host cell is a P. kudriavzevii strain.

The phosphoglycerate mutase consensus sequence #3 (SEQ ID NO: 25) provides the sequence of amino acids in which each position identifies the amino acid (if a specified amino acid is identified) or a subset of amino acids (if a position is identified as variable) most likely to be found at a specific position in a phosphoglycerate mutase. Many amino acids in SEQ ID NO: 25 are highly conserved and phosphoglycerate mutases encoded by nucleic acids suitable for deletion or disruption in accordance with the methods of the present disclosure will comprise a substantial number, and sometimes all, of these highly conserved amino acids at positions aligning with the location of the indicated amino acids in SEQ ID NO: 25. In various embodiments, proteins encoded by nucleic acids suitable for disruption in accordance with the methods of the present disclosure have phosphoglycerate mutase activity and comprise an amino acid sequence with at least 40%, at least 50%, at least 60%, or at least 70% sequence identity with SEQ ID NO: 25. For example, the Scheffersomyces stipites phosphoglycerate mutase UniProt ID: A3LN94 is 81% identical to consensus sequence #3 (SEQ ID NO: 25), and is therefore encompassed by consensus sequence #3 (SEQ ID NO: 25).

In some embodiments, recombinant host cells comprise heterologous nucleic acids encoding glyceric acid and/or glycolic acid pathway enzymes, and further comprise one or more genetic disruptions in one, more, or all phosphoglycerate mutase homologs. Example 2 describes recombinant P. kudriavzevii host cells comprising genetic disruption of the native phosphoglycerate mutase resulting in glyceric acid production whereas the wild type host cell produced no glyceric acid. Furthermore, Example 5 describes recombinant P. kudriavzevii host cells comprising heterologous nucleic acids encoding glyceric acid pathway enzymes and also comprising genetic disruption of the native phosphoglycerate mutase. Recombinant host cells comprising both genetic modifications (i.e., expressing the glyceric acid pathway enzyme and disruption of the phosphoglycerate mutase) produced more glyceric acid than recombinant host cells comprising only genetic disruption of the phosphoglycerate mutase.

In said recombinant host cells, carbon flux through glycolysis to pyruvate and native byproducts, including ethanol and/or acetate, is reduced. In certain embodiments, pyruvate, ethanol, and/or acetate byproduct titers (i.e., g of byproduct/liter of fermentation volume) at the end of fermentation are 10 g/l or less, preferably 5 g/l or less, and most preferably 2.5 g/l or less. In certain embodiments, pyruvate, ethanol, and/or acetate byproduct yield (i.e., percentage of g of byproduct/g of substrate) at the end of fermentation is 10% or less, 5% or less, 2.5% or less, and preferably, 1% or less.

### 2.5.2 Decreasing or Eliminating Expression of Phosphoglycerate Dehydrogenase

Another enzyme that depletes the cellular pool of 3-PG is phosphoglycerate dehydrogenase (EC #1.1.1.95); it catalyzes the conversion of one molecule of 3-PG and one molecule of NAD(P)⁺ to one molecule of 3-phosphonooxypypruvate and one molecule of NAD(P)H. In some cases, it is beneficial to decrease or eliminate expression of genes encoding phosphoglycerate dehydrogenase to prevent 3-PG depletion.

Several amino acids in phosphoglycerate dehydrogenase homologs are highly conserved. Any protein that is a phosphoglycerate dehydrogenase homolog will comprise a substantial number of highly conserved amino acids. For example, in S. cerevisiae, there are at least two phosphoglycerate dehydrogenase homologs: ScSER3 protein (UniProt ID: P40054; SEQ ID NO: 15) and ScSER33 (UniProt ID: P40510). ScSER33 shares substantial sequence identity with ScSER3 and is thus a protein homolog of ScSER3. In some embodiments, recombinant host cells of the present disclosure comprise one or more genetic disruptions to one or more phosphoglycerate dehydrogenase homologs with at least 40%, at least 50%, at least 60%, at least 70%, at least 80%, at least 90%, at least 95%, or more than 95% homology when compared to ScSER3 (SEQ ID NO: 15).

The phosphoglycerate dehydrogenase consensus sequence #2 (SEQ ID NO: 17) provides the sequence of amino acids in which each position identifies the amino acid (if a specified amino acid is identified) or a subset of amino acids (if a position is identified as variable) most likely to be found at a specific position in a phosphoglycerate dehydrogenase. Many amino acids in SEQ ID NO: 17 are highly conserved and phosphoglycerate dehydrogenases suitable for use in accordance with the methods of the present disclosure will comprise a substantial number, and sometimes all, of these highly conserved amino acids at positions aligning with the location of the indicated amino acids in SEQ ID NO: 17. In various embodiments, proteins suitable for use in accordance with the methods of the present disclosure have phosphoglycerate dehydrogenase activity and comprise an amino acid sequence with at least 40%, at least 50%, at least 60%, or at least 70% sequence identity with SEQ ID NO: 17. For example, the Aspergillus clavatus D-3-phosphoglycerate dehydrogenase UniProt ID: A1C9P2 is 63% identical to consensus sequence #2 (SEQ ID NO: 17), and is therefore encompassed by consensus sequence #2 (SEQ ID NO: 17).

In some embodiments, recombinant host cells comprise heterologous nucleic acids encoding glyceric acid and/or glycolic acid pathway enzymes, and further comprise one or more genetic disruptions in one, more, or all phosphoglycerate dehydrogenase homologs.

3-phosphonoxypyruvate is an intermediate in the biosynthesis of serine and in certain embodiments, serine byproduct titer (i.e., g of byproduct/liter of fermentation volume) at the end of fermentation is 10 g/l or less, preferably 5 g/l or less, and most preferably 2.5 g/l or less. In certain embodiments, serine byproduct yield (i.e., percentage of g of byproduct/g of substrate) at the end of fermentation is 10% or less, 5% or less, 2.5% or less, and preferably, 1% or less.

### 2.5.3 Decreasing or Eliminating Expression of Enolase

In some embodiments, it is beneficial to decrease or eliminate carbon flux through glycolysis (downstream of 2-PG) by decreasing or eliminating expression of enolase (EC #4.2.1.11). Enolase catalyzes the conversion of one molecule of 2-PG to one molecule of phosphoenolpyruvate and on molecule of water, diverting 2-PG from the glyceric acid and/or downstream product enolase activity, and in some of these cases, it will be necessary to grow the biomass on an alternative carbon source to glucose (for example, ethanol, acetate, pyruvate, or citrate). In other cases, it is beneficial to decrease expression of enolase such that a small amount of glucose is converted to pyruvate during the production phase of the fermentation.

Several amino acids in enolase homologs are highly conserved. Any protein that is an enolase homolog will comprise a substantial number of highly conserved amino acids. For example, in S. cerevisiae, there are least two enolase homologs: ScENO1 protein (UniProt ID: P00924; SEQ ID NO: 28) and ScENO2 (UniProt ID: P00925, SEQ ID NO: 29). ScENO1 shares substantial sequence identity with ScENO2 and is thus a protein homolog of ScENO1. In some embodiments, recombinant host cells of the present disclosure comprise one or more genetic disruptions to one or more enolase homologs with at least 40%, at least 50%, at least 60%, at least 70%, at least 80%, at least 90%, at least 95%, or more than 95% homology when compared to ScENO1 (SEQ ID NO: 28) or ScENO2 (SEQ ID NO: 29).

The enolase consensus sequence #4 (SEQ ID NO: 26) provides the sequence of amino acids in which each position identifies the amino acid (if a specified amino acid is identified) or a subset of amino acids (if a position is identified as variable) most likely to be found at a specific position in an enolase. Many amino acids in SEQ ID NO: 26 are highly conserved and enolase suitable for use in accordance with the methods of the present disclosure will comprise a substantial number, and sometimes all, of these highly conserved amino acids at positions aligning with the location of the indicated amino acids in SEQ ID NO: 26. In various embodiments, proteins suitable for use in accordance with the methods of the present disclosure have enolase activity and comprise an amino acid sequence with at least 40%, at least 50%, at least 60%, or at least 70% sequence identity with SEQ ID NO: 26. For example, the Ajellomyces capsulatus enolase UniProt ID: CONABO is 83% identical to consensus sequence #4 (SEQ ID NO: 26), and is therefore encompassed by consensus sequence #4 (SEQ ID NO: 26).

In some embodiments, recombinant host cells comprise heterologous nucleic acids encoding glyceric acid and/or glycolic acid pathway enzymes, and further comprise one or more genetic disruptions in one, more, or all enolase homologs.

In said recombinant host cells, carbon flux through glycolysis to pyruvate and native byproducts, including ethanol and/or acetate, is reduced. In certain embodiments, pyruvate, acetate, and/or ethanol byproduct titer (i.e., g of byproduct/liter of fermentation volume) at the end of fermentation is 10 g/l or less, preferably 5 g/l or less, and most preferably 2.5 g/l or less. In certain embodiments, pyruvate, ethanol, and/or ethanol byproduct yield (i.e., percentage of g of byproduct/g of substrate) at the end of fermentation is 10% or less, 5% or less, 2.5% or less, and preferably, 1% or less.

### 2.5.4 Decreasing or Eliminating Expression of Glycerate 3-Kinase

In some embodiments, it is beneficial to decrease or eliminate expression of glycerate 3-kinase (EC #2.7.1.31) to prevent glycerate from being converted back to 3-PG, reversing the 3-PG phosphatase reaction, thus leaving downstream product pathway(s). In some cases, it is advantageous to decrease or eliminate expression of genes encoding enzymes with glycerate kinase.

Several amino acids in glycerate 3-kinase homologs are highly conserved. Any protein that is a glycerate 3-kinase homolog will comprise a substantial number of highly conserved amino acids. In Escherichia coli, there is at least one glycerate 3-kinase: the GlxK protein (abbv. EcGLXK; UniProt ID: P77364; SEQ ID NO: 16.) In some embodiments, recombinant host cells of the present disclosure comprise one or more genetic disruptions to one or more glycerate 3-kinase homologs with at least 40%, at least 50%, at least 60%, at least 70%, at least 80%, at least 90%, at least 95%, or more than 95% homology when compared to EcGLXK2.

In some embodiments, recombinant host cells comprise heterologous nucleic acids encoding glyceric acid and/or glycolic acid pathway enzymes, and further comprise one or more genetic disruptions in one, more, or all glycerate 3-kinase homologs.

In said recombinant host cells, carbon flux through glycolysis to pyruvate and native byproducts, including ethanol and/or acetate, is reduced. In certain embodiments, pyruvate, ethanol, and/or acetate byproduct titers (i.e., g of byproduct/liter of fermentation volume) at the end of fermentation are 10 g/l or less, preferably 5 g/l or less, and most preferably 2.5 g/l or less. In certain embodiments, pyruvate, ethanol, and/or acetate byproduct yield (i.e., percentage of g of byproduct/g of substrate) at the end of fermentation is 10% or less, 5% or less, 2.5% or less, and preferably, 1% or less. In certain embodiments, 3-PG byproduct titer (i.e., g of byproduct/liter of fermentation volume) at the end of fermentation is 10 g/l or less, preferably 5 g/l or less, and most preferably 2.5 g/l or less. In certain embodiments, 3-PG byproduct yield (i.e., percentage of g of byproduct/g of substrate) at the end of fermentation is 10% or less, 5% or less, 2.5% or less, and preferably, 1% or less.

### 2.5.5 Decreasing or Eliminating Expression of Glycerate 2-Kinase

Another enzyme that depletes the cellular pool of glycerate is glycerate 2-kinase. In some embodiments, it is beneficial to decrease or eliminate expression of glycerate 2-kinase (EC #2.7.1.165) to prevent glycerate from being converted to 2-phosphoglycerate (2-PG), thus leaving the glycolic acid pathway. In some cases, it is beneficial to decrease or eliminate expression of genes encoding enzymes with glycerate 2-kinase activity prevent glycerate depletion.

Several amino acids in glycerate 2-kinase homologs are highly conserved. Any protein that is a glycerate 2-kinase homolog will comprise a substantial number of highly conserved amino acids. For example, in Escherichia coli, there is at least one glycerate 2-kinase homologs: the GarK protein (abbv. EcGARK; UniProt ID: P23524; SEQ ID NO: 18). In some embodiments, recombinant host cells of the present disclosure comprise one or more genetic disruptions to one or more glycerate 2-kinase homologs with at least 40%, at least 50%, at least 60%, at least 70%, at least 80%, at least 90%, at least 95%, or more than 95% homology when compared to EcGARK.

In some embodiments, recombinant host cells comprise heterologous nucleic acids encoding glyceric acid and/or glycolic acid pathway enzymes, and further comprise one or more genetic disruptions in one, more, or all glycerate 2-kinase homologs.

In said recombinant host cells, carbon flux through glycolysis to pyruvate and native byproducts, including ethanol and/or acetate, is reduced. In certain embodiments, pyruvate, ethanol, and/or acetate byproduct titers (i.e., g of byproduct/liter of fermentation volume) at the end of fermentation are 10 g/l or less, preferably 5 g/l or less, and most preferably 2.5 g/l or less. In certain embodiments, pyruvate, ethanol, and/or acetate byproduct yield (i.e., percentage of g of byproduct/g of substrate) at the end of fermentation is 10% or less, 5% or less, 2.5% or less, and preferably, 1% or less. In certain embodiments, 2-PG byproduct titer (i.e., g of byproduct/liter of fermentation volume) at the end of fermentation is 10 g/l or less, preferably 5 g/l or less, and most preferably 2.5 g/l or less. In certain embodiments, 2-PG byproduct yield (i.e., percentage of g of byproduct/g of substrate) at the end of fermentation is 10% or less, 5% or less, 2.5% or less, and preferably, 1% or less.

### 2.5.6 Decreasing or Eliminating Expression of Glycerol-3-Phosphate Dehydrogenase

In addition to the possible byproducts derived from 3-PG and glycolic acid pathway intermediates and product, additional byproducts can arise from intermediates in glycolysis (Table 2). Glycerol is a common byproduct that occurs under conditions of excess NADH. NAD-dependent glycerol-3-phosphate dehydrogenase (EC #1.1.1.8) catalyzes the conversion of one molecule of dihydroxyacetone phosphate (DHAP; glycerone phosphate) and one molecule of NAD(P)H to one molecule of glycerol 3-phosphate and one molecule of NAD(P)⁺, leading to the formation of the undesired byproduct glycerol. In P. kudriavzevii, NAD-dependent glycerol-3-phosphate dehydrogenase activity is the Gpd1 protein (abbv. PkGPD1; SEQ ID NO: 19).

Although this glycerol-3-phosphate dehydrogenase-catalyzed reaction is reversible, glycerol 3-phosphate production is favored during glyceric and glycolic acid production due to NADH accumulation in the cytosol. Decreasing or eliminating the expression of PkGPD1 or its homologs is useful for decreasing glycerol byproduct accumulation. In some embodiments of the present disclosure, recombinant host cells comprise one or more genetic disruptions in one or more nucleic acids encoding a glycerol-3-phosphate dehydrogenase that gives rise to decreased, altered or eliminated expression and/or protein activity. In embodiments where the recombinant host cell is a P. kudriavzevii strain, the glycerol-3-phosphate dehydrogenase is PkGPD1.

Several amino acids in glycerol-3-phosphate dehydrogenase are highly conserved. Any protein that is homologous to PkGPDI will comprise amino acids corresponding to a substantial number of highly conserved amino acids in PkGPD1. In some embodiments, recombinant host cells of the present disclosure comprise one or more genetic disruptions in one or more PkGPD1 homologs with at least 40%, at least 50%, at least 60%, at least 70%, at least 80%, at least 90%, at least 95%, or more than 95% homology when compared to PkGPD1.

In some embodiments, recombinant host cells comprise heterologous nucleic acids encoding glyceric acid pathway enzymes, and further comprise one or more genetic disruptions in one, more, or all PkGPD1 homologs. Glycerol-3-phosphate is an intermediate in the byproduct pathway resulting in glycerol. In certain embodiments, glycerol 3-phosphate byproduct titer at the end of fermentation is 10 g/l or less, preferably at a titer of 5 g/l or less, and most preferably at a titer of 2.5 g/l or less. In certain embodiments, glycerol 3-phosphate byproduct yield at the end of fermentation is 10% or less, 5% or less, 2.5% or less, and preferably, 1% or less. In certain embodiments, glycerol byproduct titer at the end of fermentation is 10 g/l or less, preferably at a titer of 5 g/l or less, and most preferably at a titer of 2.5 g/l or less. In certain embodiments, glycerol byproduct yield at the end of fermentation is 10% or less, 5% or less, 2.5% or less, and preferably, 1% or less.

### 2.6 Genetic Engineering

Expression of glyceric acid pathway enzymes is achieved by transforming host cells with exogenous nucleic acids encoding glyceric acid pathway enzyme(s), producing recombinant host cells of the present disclosure. The same is true for expression of downstream product pathway enzymes and ancillary proteins. Any method can be used to introduce exogenous nucleic acids into a host cell to produce a recombinant host cell of the present disclosure. Many such methods are known to practitioners in the art. Some examples comprise electroporation, chemical transformation, and conjugation. Some examples comprise electroporation, chemical transformation, and conjugation. After exogenous nucleic acids enter the host cell, nucleic acids may integrate in to the cell genome via homologous recombination.

Recombinant host cells of the present disclosure may comprise one or more exogenous nucleic acid molecules/elements, as well as single or multiple copies of a particular exogenous nucleic acid molecule/element as described herein. These molecules/elements comprise transcriptional promoters, transcriptional terminators, protein coding regions, open reading frames, regulatory sites, flanking sequences for homologous recombination, and intergenic sequences.

Exogenous nucleic acids can be maintained by recombinant host cells in various ways. In some embodiments, exogenous nucleic acids are integrated into the host cell genome. In other embodiments, exogenous nucleic acids are maintained in an episomal state that can be propagated, either stably or transiently, to daughter cells. Exogenous nucleic acids may comprise selectable markers to ensure propagation. In some embodiments, the exogenous nucleic acids are maintained in recombinant host cells with selectable markers. In some embodiments, the selectable markers are removed and exogenous nucleic acids are maintained in a recombinant host cell strain without selection. In some embodiments, removal of selectable markers is advantageous for downstream processing and purification of the fermentation product.

In some embodiments, endogenous nucleic acids (i.e., genomic or chromosomal elements of a host cell), are genetically disrupted to alter, mutate, modify, modulate, disrupt, enhance, remove, or inactivate a gene product. In some embodiments, genetic disruptions alter expression or activity of proteins native to a host cell. In some embodiments, genetic disruptions circumvent unwanted byproduct formation or byproduct accumulation. Genetic disruptions occur according to the principle of homologous recombination via methods well known in the art. Disrupted endogenous nucleic acids can comprise open reading frames as well as genetic material that is not translated into protein. In some embodiments, one or more marker genes replace deleted genes by homologous recombination. In some of these embodiments, the one or more marker genes are later removed from the chromosome using techniques known to practitioners in the art.

Section 3: Methods of Producing Glyceric Acid, Glycerate Salts, and/or Downstream Products With Recombinant Host Cells

Methods are provided herein for producing glyceric acid, glycerate salts, and/or downstream products from recombinant host cells of the present disclosure. In certain embodiments, the methods comprise the steps of: (1) culturing recombinant host cells as provided by the present disclosure in a fermentation broth containing at least one carbon source and one nitrogen source under conditions such that glycerate is produced; and (2) recovering the glycerate, glyceric acid or glycerate salt from the fermentation broth. In some embodiments, the glyceric acid is first converted to a glycerate salt before the glycerate salt is recovered from the fermentation broth. In some embodiments, the glyceric acid or glycerate salt is first converted to a downstream product before the downstream product is recovered from the fermentation broth. In some embodiments, the glyceric acid or glycerate salt is converted to a downstream product by recombinant host cells of the present disclosure. In some embodiments, the downstream products comprise glycolic acid and glycolate salt.

3.1 Fermentative Production of Glyceric Acid, Glycerate Salts, and/or Downstream Products by Recombinant Host Cells

Any of the recombinant host cells of the present disclosure can be cultured to produce and/or secret glycerate (i.e., glycerate salt and glyceric acid). In some embodiments, the recombinant host cells of the present disclosure can be cultured to also produce one or more downstream products, such as glycolate salt and glycolic acid. As disclosed herein, the glycerate or downstream product can then be esterified and distilled to generate a purified ester.

Materials and methods for the maintenance and growth of microbes, as well as fermentation conditions, are well known to practitioners of ordinary skill in the art. It is understood that consideration can be given to appropriate culture medium, pH, temperature, revival of frozen stocks, growth of seed cultures and seed trains, and requirements for aerobic, microaerobic, or anaerobic conditions, depending on the specific requirements of the host cells, the fermentation, and process flows.

The methods of producing glycerate and/or one or more downstream products provided herein may be performed in a suitable fermentation broth in a suitable bioreactor such as a fermentation vessel, including but not limited to a culture plate, a flask, or a fermenter. Further, the methods can be performed at any scale of fermentation known in the art to support microbial production of small-molecules on an industrial scale. Any suitable fermenter may be used including a stirred tank fermenter, an airlift fermenter, a bubble column fermenter, a fixed bed bioreactor, or any combination thereof.

In some embodiments of the present disclosure, the fermentation broth is any fermentation broth in which a recombinant host cell capable of producing glycerate and/or a downstream product according to the present disclosure, and can subsist (i.e., maintain growth, viability, and/or catabolize glucose or other carbon source). In some embodiments, the fermentation broth is an aqueous medium comprising assimilable carbon, nitrogen, and phosphate sources. Such a medium can also comprise appropriate salts, minerals, metals, and other nutrients. In some embodiments, the carbon source and cell nutrients are provided to the fermentation broth incrementally or continuously, and maintained at essentially the minimum level required for efficient assimilation by growing cells. Examples of cell growth procedures comprise batch fermentation, fed-batch fermentation with batch separation, fed-batch fermentation with continuous separation, and continuous fermentation with continuous separation. These procedures are well known to practitioners of ordinary skill in the art.

In some embodiments of the present disclosure, the handling and culturing of recombinant host cells to produce glycerate and/or downstream product may be divided up into phases, such as growth phase, production phase, and/or recovery phase. The following paragraphs provide examples of features or purposes that may relate to these different phases. These features or purposes may vary based on the recombinant host cells used, the desired glycerate and/or downstream product yield, titer, and/or productivity, or other factors. While it may be beneficial in some embodiments for the glyceric acid pathway enzymes and/or downstream product pathway enzymes, ancillary proteins and/or endogenous host cell proteins to be constitutively expressed, in other embodiments, it may be preferable to selectively express or repress any or all of the aforementioned proteins.

During growth phase, recombinant host cells may be cultured to focus on growing cell biomass by utilizing the carbon source provided. In some embodiments, expression of glyceric acid pathway enzymes, and/or downstream product pathway enzymes, and/or ancillary proteins are repressed or uninduced. In some embodiments, no appreciable amount of glycerate, downstream product, or any of their pathway intermediates are made. In some embodiments, proteins that contribute to cell growth and/or cellular processes may be selectively expressed.

During production phase, however, recombinant host cells may be cultured to stop producing cell biomass and to focus on glycerate and/or downstream product biosynthesis by utilizing the carbon source provided. In some embodiments, glyceric acid pathway enzymes, downstream product pathway enzymes, and/or ancillary proteins, may be selectively expressed during production to generate high product titers, yields and productivities. The production phase is synonymous with fermentation, fermentation run or fermentation phase.

In some embodiments, the growth and production phases take place at the same time. In other embodiments, the growth and production phases are separate. While in some embodiments, product is made exclusively during production phase, in other embodiments some product is made during growth phase before production phase begins.

The recovery phase marks the end of the production phase, during which cellular biomass is separated from fermentation broth and glycerate or downstream product is purified from fermentation broth. In some fermentation methods, for example, fill-draw and continuous fermentations, there may be multiple recovery phases where fermentation broth containing biomass and glycolic acid are removed from the fermentation system. The draws of fermentation broth may be processed independently or may be stored, pooled, and processed together. In other fermentation methods, for example, batch and fed-batch fermentations, there may only be a single recovery phase.

Fermentation procedures are particularly useful for the biosynthetic production of commercial glycerate and/or downstream product. Fermentation procedures can be scaled up for manufacturing glycerate and/or downstream product and examples of fermentation procedures comprise, for example, fed-batch fermentation and batch product separation; fed-batch fermentation and continuous product separation; batch fermentation and batch product separation; and continuous fermentation and continuous product separation.

### 3.1.1 Carbon Source

The carbon source provided to the fermentation can be any carbon source that can be fermented by recombinant host cells. Suitable carbon sources comprise, but are not limited to, monosaccharides, disaccharides, polysaccharides, glycerol, acetate, ethanol, methanol, methane, or one or more combinations thereof. Examples of monosaccharides suitable for use in accordance to the methods of the present disclosure comprise, but are not limited to, dextrose (glucose), fructose, galactose, xylose, arabinose, and any combination thereof. Examples of disaccharides suitable for use in accordance to the methods of the present disclosure comprise, but are not limited to, sucrose, lactose, maltose, trehalose, cellobiose, and any combination thereof. Examples of polysaccharides suitable for use in accordance to the methods of the present disclosure comprise, but are not limited to, starch, glycogen, cellulose, and combinations thereof. In some embodiments, the carbon source is dextrose. In other embodiments, the carbon source is sucrose. In some embodiments, mixtures of some or all the aforementioned carbon sources can be used in fermentation.

3.1.2 pH

The pH of the fermentation broth can be controlled by the addition of acid or base to the culture medium. Preferably, fermentation pH is controlled at the beginning of fermentation and then allowed to drop as glycolic acid accumulates in the broth, minimizing the amount of base added to the fermentation (thereby improving process economics) as well as minimizing the amount of salt formed. Specifically, the pH during fermentation is maintained in the range of 2-8, and more preferably, in the range of 4-8. At the end of fermentation, the final pH is in the range of 2-5. Non-limiting examples of suitable acids used to control fermentation pH comprise acetic acid, hydrochloric acid, and sulfuric acid. Non-limiting examples of suitable bases used to control fermentation pH comprise sodium bicarbonate (NaHCO₃), sodium hydroxide (NaOH), potassium bicarbonate (KHCO₃), potassium hydroxide (KOH), calcium hydroxide (Ca(OH)₂), calcium carbonate (CaCO₃), ammonia, ammonium hydroxide, and diammonium phosphate. In some embodiments, a concentrated acid or concentrated base is used to limit dilution of the fermentation broth.

Base cations and glycerate anions react to form ionic compounds in fermentation broths. Base cations and downstream product anions also react to form ionic compounds in fermentation broths. For example, base Na⁺ cations and glycerate anions react to form sodium glycerate; similarly, base Na⁺ cations and glycolate anions react to form sodium glycolate. In some embodiments, the ionic compounds formed by base cations and glycerate or glycolate anions are soluble in fermentation broth. In other embodiments, the ionic compounds formed by base cations and glycerate or glycolate anions are insoluble salts and may crystallize in the fermentation broth.

### 3.1.3 Temperature

The temperature of the fermentation broth can be any temperature suitable for growth of the recombinant host cells and/or production of glycerate and/or downstream product. Preferably, during glycerate and/or downstream product production, the fermentation broth is maintained within a temperature range of from about 20° C. to about 45° C., and more preferably in the range of from about 25° C. to about 42° C.

### 3.1.4 Oxygen/Aeration

Generally speaking, microbial production of glycerate and/or downstream product from glucose results in the formation of NAD(P)H, redox cofactors that are to be converted back to NAD(P)⁺ in order to maintain catabolism of glucose. Under aerobic conditions, microbes will commonly use molecular oxygen as an electron acceptor to reoxidize these cofactors. If the fermentation is not appropriately oxygenated, glycerate and/or downstream product production will decrease. During cultivation, aeration and agitation conditions are selected to produce an oxygen transfer rate (OTR; rate of dissolution of dissolved oxygen in a fermentation medium) that results in glycerate and/or downstream product formation. In various embodiments, fermentation conditions are selected to produce an OTR of greater than 10 mmol/l/hr. In some embodiment, fermentation conditions are selected to produce an OTR of greater than 20 mmol/l/hr, greater than 30 mmol/l/hr, greater than 40 mmol/l/hr, greater than 50 mmol/l/hr, greater than 75 mmol/l/hr, greater than 100 mmol/l/hr, greater than 125 mmol/l/hr, greater than 150 mmol/l/hr, greater than 175 mmol/l/hr, or greater than 200 mmol/l/hr. OTR, as used herein, refers to the volumetric rate at which oxygen is consumed during the fermentation. Inlet and outlet oxygen concentrations can be measured by exhaust gas analysis, for example by mass spectrometers. OTR can be calculated using the Direct Method described in Bioreaction Engineering Principles 3^{rd} Edition, 2011, Spring Science+Business Media, p. 449. The recombinant host cells of the present disclosure are able to produce glyceric acid and/or downstream product(s) under a wide range of oxygen concentrations.

### 3.1.5 Yields and Titers

A high yield of glycerate and/or downstream product from the provided carbon source(s) is desirable to decrease the production cost. As used herein, yield is calculated as the percentage of the mass of carbon source catabolized by recombinant host cells of the present disclosure and used to produce glycerate and/or downstream product. In some cases, only a fraction of the carbon source provided to a fermentation is catabolized by the cells, and the remainder is found unconsumed in the fermentation broth or is consumed by contaminating microbes in the fermentation. Thus, fermentation ought to be both substantially pure of contaminating microbes and that the concentration of unconsumed carbon source at the completion of the fermentation is measured. For example, if 100 grams of glucose is fed into the fermentation, and at the end of the fermentation 25 grams of glycerate is produced and there remains 10 grams of glucose, the glycerate yield is 27.7% (i.e., percentage of 25 grams glycerate produced per 90 grams glucose consumed). In certain embodiments of the methods provided herein, the final yield of glycerate on the carbon source is at least 10%, at least 20%, at least 30%, at least 35%, at least 40%, at least 50%, at least 60%, at least 70%, at least 80%, or greater than 80%. In certain embodiments, the recombinant host cells provided herein are capable of producing at least 70%, at least 75%, or greater than 80% by weight of carbon source to glycerate. When a glycerate salt is found in the fermentation broth, the glyceric acid yield can be determined by calculating the mols of glycerate salt present and adjusting for the molecular weight difference between the glycerate salt and glyceric acid.

In addition to yield, the titer (or concentration), of glycerate and/or downstream product produced in the fermentation, is another metric for production. Assuming all other metrics are equal, a higher titer is preferred to a lower titer. Generally speaking, titer is provided as grams of product (for example, glycerate or downstream product) per liter of fermentation broth (i.e., g/l). In some embodiments, the glyceric acid or glycerate salt titer is at least 1 g/l, at least 5 g/l, at least 10 g/l, at least 15 g/l, at least 20 g/l, at least 25 g/l, at least 30 g/l, at least 40 g/l, at least 50 g/l, at least 60 g/l, at least 70 g/l, at least 80 g/l, at least 90 g/l, at least 100 g/l, or greater than 100 g/l at some point during the fermentation, and preferably at the conclusion of the fermentation. As with yield calculations, a glyceric acid titer can be calculated from the glycerate salt titer by adjusting for molecular weight differences between the glycerate salt and glyceric acid.

Further, productivity, or the rate of product (i.e., glycerate or downstream product) formation, is useful in decreasing production cost, and, assuming all other metrics are equal a higher productivity is preferred over a lower productivity. Generally speaking, productivity is provided as grams product produced per liter of fermentation broth per hour (i.e., g/l/hr). In some embodiments, glyceric acid and/or downstream product productivity is at least 0.1 g/l/hr, at least 0.25 g/l/hr, at least 0.5 g/l/hr, at least 0.75 g/l/hr, at least 1.0 g/l/hr, at least 1.25 g/l/hr, at least 1.25g/l/hr, at least 1.5 g/l/hr, or greater than 1.5 g/l/hr over some time period during the fermentation.

HPLC is an appropriate method to determine the amount of glycerate or downstream product produced, the amount of any byproducts produced (for example, organic acids and alcohols), the amount of any pathway metabolite or intermediate produced, and the amount of unconsumed glucose left in the fermentation broth. Aliquots of fermentation broth can be isolated for analysis at any time during fermentation, as well as at the end of fermentation. Briefly, molecules in the fermentation broth are first separated by liquid chromatography (LC); then, specific liquid fractions are selected for analysis using an appropriate method of detection (for example, UV-VIS, refractive index, and/or photodiode array detectors). In some embodiments of the present disclosure, a salt (for example, glycerate or downstream product) is the fermentative product present in the fermentation broth. The salt is acidified before or during HPLC analysis to produce glyceric acid or corresponding organic acid of a downstream product. Hence, the acid concentration calculated by HPLC analysis can be used to calculate the salt titer in the fermentation broth by adjusting for difference in molecular weight between the two compounds.

Gas chromatography-mass spectrometry (GC-MS) is also an appropriate method to determine the amount of target product and byproducts, particularly if they are volatile. Samples of fermentation can be isolated any time during and after fermentation and volatile compounds in the headspace can be extracted for analysis. Non-volatile compounds in the fermentation medium (for example, organic acids) can also be analyzed by GC-MS after derivatization (i.e., chemical alteration) for detection by GC-MS. Non-volatile compounds can also be extracted from fermentation medium by sufficiently increasing the temperature of the fermentation medium, causing non-volatile compounds to transition into gas phase for detection by GC-MS. Molecules are carried by an inert gas carries as they move through a column for separation and then arrive at a detector.

### Section 4: Purification of Fermentation Products Glyceric Acid, Glycerates, Glycolic Acid and Glycolates

The present disclosure describes the methods for purifying fermentation product synthesized by recombinant cells of the present disclosure, wherein the fermentation product comprises glyceric acid, glycerates, glycolic acid, and glycolates. The methods comprise separating soluble fermentation product from fermentation broth, cells, cell debris and soluble impurities, and isolating the soluble fermentation product. In some examples, the methods may also comprise converting fermentation product from soluble form to insoluble, crystalline form, and isolating the crystalline fermentation product.

At the end of fermentation, the fermentation broth contains fermentation product, in soluble and/or insoluble forms, together with biomass and soluble impurities that comprise salts, proteins, unconverted sugars, and other impurities including color bodies. Biomass and soluble impurities are removed via a series of purification steps. In certain embodiments of the present disclosure, purification steps may comprise centrifugation, microfiltration, ultrafiltration, nanofiltration, diafiltration, ion exchange, crystallization, and any combination thereof. In some of these embodiments, ion exchange resins and nanofiltration membranes are used as polishing steps to remove trace amounts of soluble impurities, unconverted sugars and color bodies.

### 4.1 Removal of Cells and Cell Debris

In some embodiments, the method of purifying fermentation product (i.e., glyceric acid, glycerates, glycolic acid, and glycolates) comprises a step of separating a liquid fraction containing fermentation product from a solid fraction that contains cells and cell debris. For this separation, any amount of fermentation broth can be processed, including the entirety of the fermentation broth. The amount of fermentation broth processed can depend on the type of fermentation method used, such as batch or continuous fermentation methods. In various embodiments, removal of cells and cell debris can be accomplished, for example, via centrifugation using specific g-forces and residence times, and/or filtration using molecular weight cutoffs that are suitable for efficiently separating the liquid fraction containing fermentation product from the solid fraction that contains cells and cell debris. In some embodiments, removal of cells and cell debris is repeated at least once at one or in more than one step in the methods provided herein.

In some embodiments, centrifugation is used to provide a liquid fraction comprising fermentation product that is substantially free of cells. Many types of centrifuges useful for the removal of cells and solids from fermentation broth are known to those skilled in the art, including disc-stack and decanter centrifuges. Centrifuges are well suited for separating solids with a particle size of between 0.5 μm to 500 μm and density greater than that of the liquid phase (ca. 1.0 g/mL). Yeast cells, as a non-limiting example of a fermentation product-producing microbe, typically have a particle size between 4-6 um and a density of around 1.1 g/mL; therefore, centrifugation is well suited for removing yeast cells from fermentation broth.

In some embodiments, a disc-stack centrifuge is used to provide a liquid fraction comprising fermentation product that substantially free of cells. A disc stack centrifuge separates solids, which are discharged intermittently during operation, from liquids, typically in a continuous process. A disc-stack centrifuge is well suited for separating soft, non-abrasive solids, including cells. In some embodiments, a decanter centrifuge is used to provide a liquid fraction comprising fermentation product that is substantially free of cells. A decanter centrifuge can typically process larger amounts of solids and is often preferred over a disc-stack centrifuge for processing fermentation broth when the cell mass and other solids exceeds about 3% w/w.

Other methods can be used in addition to, or alone, with the above centrifugation processes. For example, microfiltration is also an effective means to remove cells from fermentation broth. Microfiltration includes filtering the fermentation broth through a membrane having pore sizes from about 0.5 μm to about 5 μm. In some embodiments, microfiltration is used to provide a liquid fraction comprising fermentation product that is substantially free of cells.

In some embodiments, cells removed by centrifugation and/or microfiltration are recycled back into the fermentation. Recycling cells back into the fermentation can increase fermentation product yield since less carbon source (for example, glucose) is to be used to generate new cells. Additionally, recycling cells back into the fermentation can also increase fermentation product productivity since the concentration of cells producing glycerate and/or downstream product in the fermenter can be increased.

While suitable for removing cells, centrifugation and microfiltration are not generally not effective at removing cells debris, proteins, DNA and other smaller molecular weight compounds from the fermentation broth. Ultrafiltration is a process similar to microfiltration, but the membrane has pore sizes ranging from about 0.005 μm to 0.1 μm. This pore size equates to a molecular weight cut-off (the size of macromolecule that will be ca. 90% retained by the membrane) from about 1,000 Daltons to about 200,000 Daltons. The ultrafiltration permeate will contain low-molecular weight compounds, including fermentation product and various other soluble salts while the ultrafiltration retentate will contain the majority of residual cell debris, DNA, and proteins. In some embodiments, ultrafiltration is used to provide a liquid fraction comprising glycerate salts that is substantially free of cell debris and proteins.

### 4.2 Nanofiltration and Ion Exchange Polishing of Clarified Fermentation Broth Containing Fermentation Product

In some embodiments, nanofiltration is used to separate out certain contaminating salts, sugars, color forming bodies, and other organic compounds present in clarified fermentation broth containing fermentation product (i.e., glyceric acid, glycerates, glycolic acid, and glycolates). In nanofiltration, the clarified fermentation broth (i.e., the fermentation broth resulting from the combination of centrifugation, microfiltration, and/or ultrafiltration steps described above) is filtered through a membrane having pore sizes ranging from 0.0005 μm to 0.005 μm, equating to a molecular weight cut-off of about 100 Daltons to about 2,000 Daltons. Nanofiltration can be useful for removing divalent and multivalent ions, maltose and other disaccharides (for example, sucrose), polysaccharides, and other complex molecules with a molecular weight larger than fermentation product (for example, sodium glycerate 128.059 g/mol, potassium glycerate 144.167 g/mol or calcium glycerate 286.246 g/mol). Non-limiting examples of nanofiltration materials comprise ceramic membranes, metal membranes, polymer membranes, and composite membranes.

In some embodiments, ion exchange is used to remove specific contaminating salts present in clarified fermentation broth containing fermentation product. Ion exchange elements can take the form of resin beads as well as membranes. Frequently, the resins are cast in the form of porous beads. The resins can be cross-linked polymers having active groups in the form of electrically charged sites. At these sites, ions of opposite charge are attracted but may be replaced by other ions depending on their relative concentrations and affinities for the sites. Ion exchangers can be cationic or anionic. Factors that determine the efficiency of a given ion exchange resin include the favorability for a given ion, and the number of active sites available.

A combination of nanofiltration and ion exchange steps can be combined to produce a purified solution of fermentation product from clarified fermentation broth.

### 4.3 Crystallization of Fermentation Product

Fermentation product (i.e., glyceric acid, glycerates, glycolic acid, and glycolates) purified as described thus far are crystallized to further remove water and any remaining trace, water-soluble impurities. The solution of purified fermentation product as produced by the aforementioned steps is then fed to the fermentation product crystallization step. In some embodiments of the present disclosure, the majority of one or more glycerate salts is recovered in an insoluble, crystallized form with a minor fraction of glycerate salt remaining in the mother liquor. In other embodiments, the majority of the glyceric acid is recovered in an insoluble, crystallized form with a minor fraction of glyceric acid remaining in the mother liquor.

In some embodiments of the present disclosure, the temperature of the mother liquor is changed to facilitate fermentation product crystallization. In some embodiments, the mother liquor is cooled to a temperature below 20° C. to decrease fermentation product solubility. In some these embodiments, the mother liquor is heated to evaporate excess water. In some of these embodiments, evaporative crystallization is preferred as it offers a high yield of fermentation product and prevents the formation of stable gels, which may occur if temperature is reduced below the gelling point of concentrated fermentation product solutions. In some of these embodiments, fermentation product crystallization is achieved by combining various heating and cooling steps. In some of these embodiments, supersaturation is achieved by evaporative crystallization wherein the solute is more concentrated in a bulk solvent that is normally possible under given conditions of temperature and pressure; increased supersaturation of fermentation product in the mother liquor causes the fermentation product to crystallize. Non-limiting examples of crystallizers comprise forced circulation crystallizers, turbulence/draft tube and baffle crystallizers, induced circulation crystallizers and Oslo-type crystallizers.

In some embodiments of the present disclosure, the aforementioned heating step, cooling step and change in pH are combined in various ways to crystallize fermentation product, and modified as needed, as apparent to practitioners skilled in the art.

In some embodiments, insoluble fermentation product crystals (for example, calcium glycerate or calcium glycolate) is formed during the fermentation and the resulting product crystals are isolated. Fermentation product crystals can be isolated from the mother liquor by any technique apparent to those of skill in the art. In some embodiments of the present disclosure, fermentation product crystals are isolated based on size, weight, density, or combinations thereof. Fermentation product crystal isolation based on size can be accomplished, for example, via filtration, using a filter with a specific particle size cutoff. Fermentation product crystal isolation based on weight or density can be accomplished, for example, via gravitational settling or centrifugation, using, for example, a settler, decanter centrifuge, disc-stack centrifuge, basket centrifuge, or hydrocyclone wherein suitable g-forces and settling or centrifugation times can be determined using methods known in the art. In some embodiments in which the fermentation product is calcium glycerate, the calcium glycerate crystals are isolated from the mother liquor via settling for from 30 minutes to 2 hours at a g-force of 1. In other embodiments in which the fermentation product is calcium glycerate, the calcium glycerate crystals are isolated from the fermentation broth via centrifugation for 20 seconds to 60 seconds at a g-force of from 275 x-g to 1,000 x-g.

Following isolation from the mother liquor, fermentation product crystals are wet with residual mother liquor that coats the crystals. Thus, it is useful to wash the fermentation product crystals with water to remove these trace impurities that may be in the mother liquor. When washing fermentation product crystals, it is useful to minimize the dissolution of isolated crystals in the wash water; for this reason, cold wash (around 4° C.) water is generally used. Additionally, it is useful to minimize the amount of wash water used to minimize crystal dissolution. In many embodiments, less than 10% w/w wash water is used to wash the fermentation product crystals.

In some embodiments, the methods further comprise the step of removing impurities from fermentation product crystals. Impurities may react with fermentation product crystals and reduce final yields, or contribute to fermentation product crystals of lesser purity that limits industrial utility. Non-limiting examples of impurities comprise acetic acid, succinic acid, malic acid, ethanol, glycerol, pyruvic acid, citric acid, and propionic acid. In some embodiments, removal of such impurities is accomplished by dissolving the isolated fermentation product crystals into an aqueous solution and recrystallizing the fermentation product. A non-limiting example of dissolving and recrystallizing fermentation product crystals can comprise dissolving the fermentation product in water and evaporating the resulting aqueous solution (as mentioned above), and finally re-isolating the fermentation product crystals by filtration and/or centrifugation. None, one, or more than one cycle of fermentation product recrystallization may be used so long as the resulting fermentation product are of suitable quality. In some embodiments, no fermentation product recrystallizations are performed. In other embodiments, one fermentation product recrystallization is performed. In still further embodiments, more than one fermentation product recrystallization is performed.

In some embodiments of the present disclosure, fermentation product crystals are dewatered using a combination of screening and drying methods apparent to practitioners skilled in the art. In some of these embodiments, crystal dewatering steps comprise centrifugation, belt drying, filtration, application of vacuum, or a combination thereof. In some of these embodiments, vacuum is applied at 20 mm of Hg below atmospheric pressure. Suitable devices for crystal dewatering may include a Horizontal Vacuum Belt Filter (HVBF) or a Rotary Drum

Vacuum Filter (RDVF). Fermentation product crystal isolation based on size can be accomplished, for example, via filtration, using, for example, a filter press, candlestick filter, or other industrially used filtration system with appropriate molecular weight cutoff. Fermentation product crystal isolation based on weight or density can be accomplished, for example, via gravitational settling or centrifugation, using, for example, a settler, decanter centrifuge, disc-stack centrifuge, basket centrifuge, or hydrocyclone, wherein suitable g-forces and settling or centrifugation times can be determined using methods known in the art.

In some embodiments of the present disclosure, fermentation products are crystallized in the fermentation broth prior to removal of cells, cell debris, contaminating salts and various soluble impurities. In many of these embodiments, the fermentation product crystals are separated from fermentation broth, cells, cell debris, contaminating salts and various soluble impurities by sedimentation, centrifugation, ultrafiltration, nanofiltration, ion exchange, or any combination thereof.

### Section 5: Esterification of Glycerate Salts and Recovery of Esterification Products

### 5.1 Esterification of Glycerate Salt for Glycerate Ester Production

This disclosure provides methods for esterification of glycerate using supercritical carbon dioxide (sCO2) and alcohol to produce a glycerate ester.

The method for esterification of glycerate comprises incubating a reaction mixture for a sufficient period of time to allow for formation of one or more esterification products, wherein the reaction mixture comprises: (1) a glycerate salt; (2) sCO₂; (3) a catalyst; and (4) an alcohol co-solvent. In some embodiments, the esterification method is repeated one or more times. In some embodiments, the esterification method further comprises a step that separates the glycerate ester product from leftover reaction components, reaction intermediates, and/or byproducts. In some embodiments, the esterification method is performed using a one-pot synthesis strategy.

In some embodiments, the glycerate salt is purified from recombinant host cells. In some embodiments, the glycerate salt is dissolved, dried, dewatered, and/or concentrated before esterification. In many embodiments, the glycerate salt is sodium glycerate. In other embodiments, the glycerate salt is calcium glycerate.

This disclosure provides methods for esterification of glycerate using an alcohol and esterification catalyst (see, for example, Section 5 of the present disclosure). In some embodiments, the esterification catalyst is selected from the group consisting of one or more mineral acids, organic acids, and solid acid catalyst. Examples of mineral acid catalysts that can be used in accordance with the methods of this disclosure comprise, but are not limited to, sulfuric acid, hydrochloric acid, nitric acid, phosphoric acid, boric acid, hydrofluoric acid, or hydrobromic acid. In some embodiments, the mineral acid catalyst is sulfuric acid (H₂SO₄). Examples of organic acid catalysts that can be used in accordance with the methods of this disclosure comprise, but are not limited to, formic acid and acetic acid. In some embodiments, the organic acid catalyst is acetic acid. An example of a solid acid catalyst useful accordance with the methods of this disclosure is a solid acid catalyst such as Amberlyst-15. In some embodiments, the solid acid catalyst is an acidic ion exchange resin.

In some embodiments, the co-solvent is an esterification alcohol. In some embodiments, the co-solvent is selected from the group comprising methanol, 1-propanol, 2-propanol, 1-butanol, isobutanol, 1-hexanol, 3-pentanol, and ethanol (EtOH). In some embodiments, the co-solvent(s) is recovered from the downstream distillation step and reused in the esterification step.

The reaction can be carried out in a distillation column, a jacketed glass reactor with a condenser, a Parr stirred reactor system, or other appropriate reaction vessel as deemed appropriate by skilled practitioners in the art. During the glycerate salt dissolution and esterification processes, the reaction mixture is often mixed.

In some embodiments of the present disclosure, the esterification conditions comprise a temperature within the range of 150° C. to 300° C., and a reaction pressure within the range of 1 atm to 10 atm. In some embodiments, the esterification conditions comprise a temperature that does not exceed the boiling point of the esterification alcohol. In some embodiments, the esterification conditions comprise a temperature greater than 25° C.

In some embodiments, the esterification conditions comprise a pressure within the range of 500 psi to 2,000 psi, depending on the temperature.

### 5.2 Distillation of Glycerate Esters

In various embodiments of the present disclosure, the glycerate ester is purified via distillation from esterification reaction mixtures (filtered or unfiltered), which are both described above. The reaction mixtures are distilled according to methods known to practitioners skilled in the art to separate unconverted esterification alcohol and partially converted species.

In some embodiments, distillation temperature is maintained within the range of 50° C. to 300° C., depending on the pressure. In some embodiments, distillation temperature is maintained within the range of 50° C. to 100° C. Distillation temperature refers to the temperature measured at the bottom of the distillation vessel.

In some embodiments, the distillation pressure is maintained within the range of 0 Torr to 100 Torr, depending on the temperature. In some embodiments, distillation pressure is maintained within the range of 2 kPa to 6 kPa.

Purified glycerate esters can be analyzed by a variety of methods known to practitioners skilled in the art, including HPLC (high-performance liquid chromatography) and GC-MS (gas chromatography mass spectrometry). Chromatography methods are useful for reporting the percentage product purity as a percentage of total measured organic compounds. In some embodiments, glycerate ester purity as a percentage of total organic compounds is greater than 95%. In some embodiments, glycerate ester purity as a percentage of total organic compounds is greater than 98%. In some embodiments, glycerate ester purity as a percentage of total organic compounds is greater than 99%. In some embodiments, glycerate ester purity as a percentage of total organic compounds is greater than 99.5%. In some embodiments, glycerate ester purity as a percentage of total organic compounds is greater than 99.8%.

In addition to chromatographic measurement of purity, a useful measure of product quality is water content and Karl Fischer titration can be used to determine the water content of purified glycerate ester. In some embodiments, the amount of amount of water present in the purified glycerate ester solution is less than 0.1%. In some embodiments, the amount of amount of water present in the purified glycerate ester solution is less than 0.05%.

Another useful measure of product purity is color. The presence or absence of color in purified glycerate ester solution can be measured at the end of distillation, and over a period of time when the product is in storage. Color measurements can be made using a colorimeter such as a HunterLab colorimeter. For near white solutions, the presence or absence of color can be reported according to the Yellowness Index (YI) with calculations according to the ASTM E313 standard. A change in YI over time is a measure of color formation over time. In some embodiments of the present disclosure, YI of purified glycerate ester at the end of distillation is low. In some embodiments, YI of purified glycerate ester remains substantially the same over time. In some embodiments, YI of purified glycerate ester does not change significantly over time. In some embodiments, YI of purified glycerate ester reflects desirable visual and/or optical/photonic properties.

### Section 6: Deoxydehydration of Glycerate Ester and Recovery of Deoxydehydration Products

The disclosure provides methods for deoxydehydration of glycerate esters to generate acrylate ester products. In a single step, catalytic deoxydehydration removes two hydroxyl groups from vicinal diols, providing alkene functionality with concomitant release of one molecule water as well as oxidation of an alcohol. Glycerates, and in particular glycerate esters, are attractive substrates for deoxydehydration since terminal diols and cis-vicinal diols are more reactive than internal diols and trans-diols. When applied toward a glycerate ester, deoxydehydration provides the cognate acrylate ester, sought after chemicals of great industrial importance. For example, methyl glycerate, ethyl glycerate, butyl glycerate, and 2-ethylhexyl glycerate are transformed into methyl acrylate, ethyl acrylate, butyl acrylate, and 2-ethylhexyl acrylate, respectively, by deoxydehydration. Thus, the present disclosure provides methods to convert glyceric acid, and in particular fermentation-derived glyceric acid, into acrylate esters.

The general method for deoxydehydration of glycerate esters comprises incubating a reaction mixture for a sufficient period of time to allow for formation of one or more deoxydehydration products, wherein the reaction mixture comprises: (1) a glycerate ester reactant; (2) a transition metal-based catalyst; (3) a reducing agent; (4) a solvent; (5) optionally, an acid; and (6) optionally, a gas. In many cases the reducing agent and the solvent are the same. In many embodiments, the glycerate ester reactant is selected from the non-limiting group comprising methyl glycerate, ethyl glycerate, butyl glycerate, and 2-ethylhexyl glycerate. In some embodiments, the glycerate ester is methyl glycerate. In some embodiments, the glycerate ester is ethyl glycerate. In some embodiments, the glycerate ester is butyl glycerate. In yet still further embodiments, the glycerate ester is 2-ethylhexyl glycerate. One or more glycerate esters can be included in the same deoxydehydration reaction mixture to generate a corresponding mixture of acrylate ester products. Post deoxydehydration, the different acrylate esters can then be separated, using, for example, fractional distillation, to generate individual, purified acrylate esters.

The catalysts used in the deoxydehydration reaction are transition metal-based catalysts. In many embodiments, the transition metal-based catalyst is selected from the group comprising molybdenum compounds, rhenium compounds, ruthenium compounds, and vanadium compounds. In many of these embodiments, the transition metal-based catalyst is an oxidized molybdenum, oxidized rhenium, oxidized ruthenium, and/or oxidized vanadium compound. The deoxydehydration catalyst can be homogenous or heterogeneous. In many cases, it is advantageous for the deoxydehydration catalyst to be heterogeneous to ease the separation of the catalyst from the reaction mixture.

In some embodiments, the deoxydehydration catalyst is a rhenium compound. In some of these embodiments, the rhenium compound is an oxidized rhenium compound selected from the non-limiting group comprising methyltrioxorhenium (MTO; CH₃ReO₃), HReO₄, KreO₄, Re₂(CO)₁₀, BrRe(CO)₅, and NH₄ReO₄. In particular embodiments, the deoxydehydration catalyst is MTO. Since MTO is a crystalline solid with a simple ligand free structure, it is a particularly advantageous rhenium catalyst. In particular embodiments, the deoxydehydration catalyst is NH₄ReO₄.

In some embodiments, the deoxydehydration catalyst is a molybdenum compound. As compared to rhenium, molybdenum is more abundant, and as such it both less expensive and has lower volatility in price. In some of these embodiments, the molybdenum compound is an oxidized molybdenum compound selected from the non-limiting group comprising (NH₄)₆Mo₇O₂₄·4H₂O (AHM), MoO₂Cl₂, MoO₂(CH₃)₂, MoO₂Cl₂, MoO₂Br₂, or (Bu₄N)₂Mo₆O₁₉. In specific embodiments, the deoxydehydration catalyst is AHM, a particularly cheap and commercially available catalyst.

In some embodiments, the deoxydehydration catalyst is a vanadium compound. In some of these embodiments, the vanadium compound is an oxidized vanadium compound selected from the non-limiting group comprising NH₄VO₃ (ammonium metavanadate), (nBu₄N)[VO₂(N-(2-benzylidene) thiophene-2-carbohydrazide)], and [Bu₄N][VO₂(pyridine-2,6-dicarboxylate)].

In particular embodiments, the catalyst is a ruthenium compound. In some of these embodiments, the ruthenium compound is (Cp*Ru(CO)₂)₂.

Catalyst cost is a factor in development of a low-cost synthetic deoxydehydration process. Generally speaking, it is desirable to run the reaction with low catalyst loading (i.e., the molar percentage of catalyst to reactant). In many embodiments, the catalyst loading is less than 10% (percent mol-catalyst per mol-reactant). In some embodiments, catalyst loading is less than 5%. In yet still further embodiments, catalyst loading is less than 2.5%.

A reducing agent is required in the reaction mixture that donates electrons to the glycerate ester. Any reducing agent is suitable for use in accordance with the methods of this disclosure so long as the glycerate ester can be reduced to the corresponding acrylate ester. Often times the reducing agent is also used as the solvent, and in these cases, it is advantageous for the glycerate ester to be reasonably soluble in the reducing agent. Primary and secondary alcohols make, generally speaking, good reductants and solvents for transformation of glycerate esters to acrylate esters.

In some embodiments, the reducing agent is selected from the group comprising PPh₃, H₂, Na₂SO₃, benzene, toluene, 5-nonanol, 3-octanol, 2-octanol, 1-butanol, 3-pentanol, 2-methyl-1-butanol, isopropanol, and 2-ethylhexanol. In some embodiments, the reducing agent is H₂. In many embodiments, the reducing agent is an alcohol. Small alcohols are generally ineffective reductants and four carbon or higher chain length alcohols provide higher product yields as compared to alcohols less than three carbons. For this reason, n-butanol and 2-ethylhexanol are particularly attractive reducing agents; additionally, the two alcohols are relatively inexpensive, are good solvents in which glycerate esters are soluble, and the corresponding acrylate esters, butyl acrylate and 2-ethylhexyl acrylate, are articles of commerce. In some embodiments, the reductant is n-butanol. In other embodiments, the reductant is 2-ethylhexanol.

When the reductant included in the deoxydehydration reaction mixture is a primary alcohol it is often times advantageous for the alkyl group of the glycerate ester to be the same as the primary alcohol used as the reductant. For example, when the reductant is 1-butanol the reactant is butyl glycerate. This can reduce the formation of unwanted acrylate ester byproducts generated when the two alcohol groups are different. In some embodiments when the glycerate ester is butyl glycerate, the reductant is 1-butanol and the product is butyl acrylate. In some embodiments where the glycerate ester is 2-ethylhexyl glycerate, the reductant is 2-ethylhexyanol and the product is 2-ethylhexyl acrylate.

The deoxydehydration reaction can be performed at any temperature so long as the reactant is converted to its cognate reduced product at appreciable yields. Maintaining the deoxydehydration reaction temperature at above ambient temperature, however, is useful for increasing the reaction rate and often times the product yield. The temperature of the reaction mixture may be held at an elevated temperature for some or all of the reaction period. In some instances, and in particular when the reactant is heat labile, reaction yields can be increased by running the reaction at a lower temperature for a period of time and then raising the reaction temperature to complete the reaction (i.e., increase the percentage conversion of reactant to product). In some embodiments, the temperature of the reaction mixture is above 25° C. In some embodiments, the temperature of the reaction mixture ranges from 150° C. to 300° C.

In some embodiments, the solvent is selected from the group comprising benzene, chlorobenzene, ethanol, methanol, butanol, isopropyl alcohol, 2-ethylhexanol, and 3-pentanol.

In some embodiments, the gas is selected from the group comprising nitrogen, argon, or hydrogen. In some embodiments, the deoxydehydration reaction is carried out in air. In some embodiments, the deoxydehydration reaction is carried out in nitrogen.

In some embodiments, the reaction time ranges from 1 hour to 24 hours.

In some embodiments, the deoxydehydration reaction is carried out at atmospheric pressure. In some embodiments, the deoxydehydration reaction is carried out with the pressure range of 50 psi to 300 psi.

In some embodiments, the deoxydehydration method is repeated one or more times. In some embodiments, the deoxydehydration method further comprises a step that separates the product from leftover reaction components, reaction intermediates, and/or byproducts. In some embodiments, the deoxydehydration method is performed using a one-pot synthesis strategy.

### Section 7: Poly(Glycerate Carbonate) Compounds and Methods

In one aspect, provided herein is a poly (glycerate carbonate) compound comprising repeat units of formula (I) or a salt thereof:

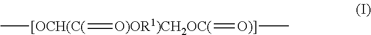

wherein each R¹ is independently hydrogen, optionally substituted C1-C5 alkyl, and optionally substituted aryl.

In one embodiment, the compound provided comprises repeat units of formula (IA): —[OCH(C(═O)OCH₃)CH₂OC(═O)]— (IA). In another embodiment, the compound provided comprises repeat units of formula (IB): —[OCH(C(═O)OC₂H₅)CH₂OC(═O)]— (IB). In another embodiment, the compound provided comprises repeat units of formula (IC): —[OCH(C(═O)OC₃H₇)CH₂OC(═O)]—(IC). In another embodiment, the compound provided comprises repeat units of formula (ID): —[OCH(C(═O)OCH₉)CH₂OC(═O)]— (ID).

The disclosure provides methods for conversion of glyceric acid, glycerate esters, and glycerate epoxides to generate poly (glycerate carbonate) compounds. In one route, glyceric acid is esterified with one or more alcohols to generate the corresponding glycerate ester(s) which is subsequently reacted with carbon dioxide to generate the poly (glycerate carbonate) compound. In a second, alternative route, the vicinal diol of the glycerate ester(s) is converted to an epoxide, and the resulting glycidate epoxide is reacted with carbon dioxide to generate the poly(glycerate carbonate) compound.

The general method for esterification of glyceric acid comprises incubating a reaction mixture for a sufficient period of time to allow for formation of one or more esterification products, wherein the reaction mixture comprises: (1) glyceric acid; (2) a catalyst; and, (3) an alcohol reactant.

In many embodiments, the alcohol reactant is selected from the non-limiting group comprising methanol, ethanol, 1-propanol, 1-butanol, and benzyl alcohol. In some embodiments, the alcohol reactant is methanol and the glycerate ester is methyl glycerate. In some embodiments, the alcohol reactant is ethanol and the glycerate ester is ethyl glycerate. In some embodiments, the alcohol reactant is 1-propanol and the glycerate ester is propyl glycerate. In some embodiments, the alcohol reactant is 1-butanol and the glycerate ester is butyl glycerate. In yet still further embodiments, the alcohol reactant is 2-ethylhexanol and the glycerate ester is 2-ethylhexyl glycerate. One or more alcohol reactants can be included in the same esterification reaction mixture to generate a corresponding mixture of glycerate ester products. Post esterification, the different glycerate esters can then be separated, using, for example, fractional distillation, to generate individual, purified glycerate esters.

In one embodiment, a poly(glycerate carbonate) compound provided herein comprises carbon atoms all of which are non-petrochemical based carbons or having ¹⁴C amounts substantially higher than zero such as about 0.5 or 1 parts per trillion or more. In one embodiment, a poly(glycerate carbonate) compound provided herein comprises carbon atoms substantially all of which are non-petrochemical based carbons or having ¹⁴C amounts substantially higher than zero such as about 0.5 or 1 parts per trillion or more.

In one embodiment, the poly (glycerate carbonate) compounds provided herein comprises an alkyl glycerate moiety which comprises a carbon-backbone having all carbon atoms that are non-petrochemical based carbons or having ¹⁴C amounts substantially higher than zero such as about 0.5 or 1 parts per trillion or more. In another embodiment, the poly (glycerate carbonate) compounds provided herein comprise an alkyl glycerate moiety which comprises a carbon-backbone having substantially all carbon atoms that are non-petrochemical based carbons or having ¹⁴C amounts substantially higher than zero such as about 0.5 or 1 parts per trillion or more

In one embodiment, the poly (glycerate carbonate) compounds provided herein comprises a ¹⁴C amount substantially higher than zero, such as about 0.5, or 1 parts per trillion or more.

In some embodiments, the method comprises an alkyl glycerate moiety which comprises a carbon-backbone having all or substantially all carbon atoms that are non-petrochemical based carbons or having ¹⁴C amounts substantially higher than zero such as about 0.5 or 1 parts per trillion or more.

In another aspect, provided herein is a poly(glycerate carbonate) compound provided by the methods provided herein.

In another aspect, provided herein is an article or manufacture comprising the poly (glycerate carbonate) compound provided herein. In some embodiment, the article of manufacture is superabsorbent polymer.

In some embodiments, the poly(glycerate carbonate) provided herein can be extruded to form fibers or films. Such techniques are well known. The fibers can be twisted to form yarns which can be woven into fabrics which are useful in the manufacture of wearing apparel and for numerous other purposes. The films can be employed for the wrapping of various packaged products and for use as a dielectric in the manufacture of electrical condensers, electric motors, transformers, etc.

In some embodiments the poly(glycerate carbonate) provided herein can be converted into a hydrogel. The hydrogel properties can be tailored by controlling degree of cross linking and functional side groups and employed in applications such as hygiene, drug delivery, tissue engineering, food additives, wound dressings and biomedical applications.

In another aspect, the poly(glycerate carbonate) compounds provided herein are degradable through hydrolytic or biological action to provide functional or stimuli-responsive degradation at end-of-life and avoid accumulation of harmful materials in the environment.

In another aspect, the poly(glycerate carbonate) compounds provided herein are bioabsorbable to provide functional or stimuli-responsive degradation in-vivo after performing the desired purpose such as drug release or providing a scaffold for in-growth of tissue with.

### Section 8:

### EXAMPLES

### Parent Strain Used in the Examples

The parent strain in Example 1 was a P. kudriavzevii strain auxotrophic for histidine and uracil due to genetic disruptions in URA2 and HIS3 (i.e., the strain cannot grow in media without histidine and uracil supplementation). Histidine auxotrophy in the parent strain enables selection of new, engineered strains that carry a HIS3 marker, enabling histidine prototrophy and indicating desired nucleic acid modification. Likewise, uracil auxotrophy in the parent strain enables selection of new, engineered strains that carry a URA2 marker, enabling uracil prototrophy and indicating desired nucleic acid modification. Thus, cells that were successfully modified with exogenous nucleic acids to comprise desired genetic modifications can grow in media without histidine and/or uracil supplementation, dependent on the selection marker included in the exogenous nucleic acid. Following confirmation of correct strain engineering, the selection marker(s) were removed by, for example, homologous recombination and marker loopout. Removing the marker enables subsequent rounds of strain engineering using the same selection markers.

### Media Used in the Examples

Complete supplement mixture (CSM) medium. CSM medium comprised Adenine 10 mg/L; L-Arginine HCl 50 mg/L; L-Aspartic Acid 80 mg/L; L-Histidine HCl 20 mg/L; L-Isoleucine 50 mg/L; L-Leucine 100 mg/L; L-Lysine HCl 50 mg/L; L-Methionine 20 mg/L; L-Phenylalanine 50 mg/L; L-Threonine 100 mg/L; L-Tryptophan 50 mg/L; L-Tyrosine 50 mg/L; Uracil 20 mg/L; L-Valine 140 mg/L. The YNB used in the CSM comprised Ammonium sulfate 5.0 g/L, Biotin 2.0 μg/L, Calcium pantothenate 400 μg/L, Folic acid 2.0 μg/L, Inositol 2.0 mg/L, Nicotinic acid 0-400 μg/L, p-Aminobenzoic acid 200 μg/L, Pyridoxine HC1 400 μg/L, Riboflavin 200 μg/L, Thiamine HCl 400 μg/L, Boric acid 500 μg/L, Copper sulfate 40 μg/L, Potassium iodide 100 μg/L, Ferric chloride 200 μg/L, Manganese sulfate 400 μg/L, Sodium molybdate 200 μg/L, Zinc sulfate 400μg/L, Potassium phosphate monobasic 1.0 g/L, Magnesium sulfate 0.5 g/L, Sodium chloride 0.1 g/L, and Calcium chloride 0.1 g/L.

Complete supplement mixture minus histidine (CSM-His) medium. CSM-His medium is identical to CSM medium with the exception that histidine was not included in the medium. Engineered strains auxotrophic for histidine are unable to grow on CSM-His medium while engineered strains containing exogenous nucleic acids comprising a histidine selectable marker (for example, HIS3) are capable of growth in CSM-His medium.

Complete supplement mixture minus uracil (CSM-Ura) medium. CSM-Ura medium is identical to CSM medium with the exception that uracil was not included in the medium. Engineered strains auxotrophic for uracil are unable to grow on CSM-Ura medium while engineered strains containing exogenous nucleic acids comprising a uracil selectable marker (for example, URA2) are capable of growth in CSM-Ura medium.

BM02 medium. BM02 medium is Glucose 125 g/l, K₂SO₄ 0.816 g/l, Na₂SO₄ 0.1236, MgSO₄-7H₂O 0.304 g/l, Urea 4.3 g/l, Myo-inositol 2 mg/l, Thiamin HC1 0.4 mg/l, Pyridoxal HCl 0.4 mg/l, Niacin 0.4 mg/l, Ca-Pantothenate 0.4 mg/l, Biotin μg/1, Folic acid 2 μg/1, PABA 200 μg/l, Riboflavin 200 μg/l, Boric acid 0.25 mg/l, Copper sulfate pentahydrate 393 μg/1, Iron sulfate 11.0 mg/l, Manganese chloride 1.6 mg/l, Sodium molybdate 100 μg/1, Zinc sulfate 4 mg/l, and EDTA 11 mg/l.

BM02-P medium. BM02-P medium is BM02 medium with 1 g/l potassium phosphate.

YPE medium. YPE medium is Bacto peptone 20 g/l, Yeast extract 10g/l, and Ethanol 2% (v/v).

### Example 1

### Construction of the Recombinant P. kudriavzevii Strain LPK151290 With Eliminated Expression of Phosphoglycerate Mutase

This example describes the construction of the P. kudriavzevii strain LPK151290 that comprises genetic disruption in both native copies of phosphoglycerate mutase, resulting in eliminated expression of phosphoglycerate mutase. The endogenous Gpm1 gene that encodes for phosphoglycerate mutase in P. kudriavzevii (PkGPM1; SEQ ID NO: 14) was disrupted to prevent the conversion of 3-PG to 2-PG in the glycolytic pathway. The deliberate, consequential buildup of 3-PG provided ample 3-PG substrate for the glyceric acid pathway, which was constructed as described in Example 3 (see below).

The parent P. kudriavzevii strain used in this example was LPK15775-it was diploid and had two copies of Gpm1, providing a GPM plus phenotype (i.e., native phenotype with respect to Gpm1 expression). The parent LPK15775 strain was also auxotrophic for uracil and histidine. Gpm1 in LPK15775 was disrupted by deletion of both gene copies to produce the GPM minus strain LPK151290.

Both copies of Gpm1 in LPK15775 were disrupted by insertion of a HIS3 selectable marker via homologous recombination. The HIS3 selectable marker, amplified by PCR, was provided to the parent LPK15775 strain to complement the histidine auxotrophic deficiency. The HIS3 selectable marker comprised unique upstream and downstream homologous regions for homologous recombination at the P. kudriavzevii Gpm1 locus, a transcriptional promoter, a HIS3 coding region, and a transcriptional terminator. The transcriptional promoter 5′ of HIS3 was the P. kudriavzevii TEF1 promoter (pPKTEF1) and the transcriptional terminator 3′ of HIS3 was the S. cerevisiae TDH3 terminator (tScTDH3).

The PCR product of the HIS3 selectable marker was purified and provided as exogenous nucleic acids to P. kudriavzevii. Transformation was carried out in a single step and gene deletion was achieved by homologous recombination. Transformants were selected on CSM-His medium and successful deletion of both copies of the gene encoding PkGPM1 was confirmed by genetic sequencing of this locus and the flanking regions.

This example produced the GPM minus, LPK151290 strain that cannot convert 3-PG to 2-PG via PkGPM1. Thus, all engineered P. kudriavzevii strains derived from this parental strain had the GPM minus phenotype, resulting in disrupted carbon flux through glycolysis downstream of 3-PG.

### Example 2

### Recombinant P. kudriavzevii Strain LPK151290 Produces Low Amounts of Glyceric Acid

This example describes the culturing and analysis of the GPM minus strain, LPK151290 (from Example 1), for basal level glyceric acid production before glyceric acid pathway strains were constructed (see Example 3 below). Recall that the parent strain of LPK151290 is LPK15775 (see Example 1). The parent LPK15775 strain expressed native levels of PkGPM1 (i.e., GPM plus) and was also cultured and analyzed for comparison.

In this example, LPK151290 (GPM minus) and LPK15775 (GPM plus) colonies were used to inoculate replicate tubes of 2 mL to 4 mL of YPE medium with 1% glycerol and incubated at 30° C. with shaking at 250 rpm for 20 hours. These replicate tubes of pre-cultures were each pelleted and resuspended in 0.5 mL of BM02-P media with 5% glucose, 1% ethanol and 50 μg/mL uracil, and placed into 96-well plates. The 96-well plate cultures were then incubated at 30° C. with 80% humidity and shaking at 250 rpm. After 48 hours, 1× volume of 12 M HCl was added to 10× volume of culture for each culture sample. The HCl-culture sample mixtures were spin-filtered and frozen for storage. Samples were analyzed by HPLC within 48 hours of harvest.

For HPLC analysis, frozen samples were thawed analyzed by HPLC using a Bio-Rad Aminex 87H column (300×7.8 mm) and a Bio-Rad Fermentation Monitoring column (#1250115; 150×7.8 mm) installed in series, with an isocratic elution rate of 0.8 ml/min with water at pH 1.95 (with sulfuric acid) at 30° C. Refractive index and UV 210 nm measurements were acquired for 35 minutes.

While the LPK15775 GPM plus strain did not produce detectable amounts of glyceric acid, the LPK151290 GPM minus strain produced a yield of 6% (i.e., percentage of the amount of glyceric acid produced per amount of glucose consumed). This result demonstrated that disruption of GPM activity alone was sufficient to produce detectable, albeit low amounts of glyceric acid in a P. kudriavzevii strain background, and more generally in microbes. Thus, all engineered P. kudriavzevii strains without heterologous nucleic acids that encode the glyceric acid pathway of the present disclosure can produce appreciable amounts of glyceric acid. Incorporation of heterologous nucleic acids that encode the glyceric acid pathway should increase glyceric acid yields (Example 4).

### Example 3

### Analysis of Strains LPK15775 (GPM Plus) and LPK151290 (GPM Minus) for the Formation of Byproducts Pyruvate, Ethanol, and Acetate

Example 3 describes the culturing and analysis of LPK15775 (GPM plus; parent P. kudriavzevii strain) and LPK151290 (GPM minus; from Example 1) recombinant host cells for in vivo production of byproducts pyruvate, ethanol and acetate. Native P. kudriavzevii cells are capable of producing downstream metabolites pyruvate, ethanol and acetate. The presence of these metabolites indicates that 3-PG is siphoned from the glyceric acid pathway of the present disclosure, thereby causing decreased fermentation yield of glyceric acid and/or downstream product.

In this example, LPK15775 and LPK151290 are cultured and analyzed by HPLC as described above in Example 2. While the GPM plus LPK15775 produce low amounts of pyruvate, ethanol and acetate, the GPM minus LPK151290 produce relatively lower amounts of pyruvate, ethanol and acetate. This example demonstrates, in accordance with the present disclosure, the successful decrease of byproducts ethanol and acetate from fermentation by genetic disruption of PkGPM in recombinant P. kudriavzevii for glyceric acid and/or downstream product synthesis.

### Example 4

### Construction of Recombinant P. kudriavzevii Strains Expressing Glyceric Acid Pathway Enzymes With Decreased or Eliminated Expression of Phosphoglycerate Mutase

Example 4 describes the construction of recombinant host cells of the present disclosure that each comprise heterologous nucleic acids encoding a glyceric acid pathway of the present disclosure. The parent P. kudriavzevii strain LPK15775 used in this example was auxotrophic for uracil and histidine. Heterologous nucleic acids encoding 3-PG phosphatases of the glyceric acid pathway were integrated into the PkGPM1 locus. Thus, each strain described in this example not only comprised a glyceric acid pathway, but also comprised a GPM minus phenotype due to eliminated expression of phosphoglycerate mutase.

The heterologous nucleic acids used in this example encoded 3-PG phosphatases of the glyceric acid pathway that were derived from EcGPH; SEQ ID NO: 1, HsGPH; SEQ ID NO: 3, ScPHO13; SEQ ID NO: 9, PKPHO13; SEQ ID NO: 10, PKORF64; SEQ ID NO: 11, PKORF423; SEQ ID NO: 12, or ScYKR070W; SEQ ID NO: 13. Heterologous nucleic acids encoding HsGPH were codon-optimized for yeast and were synthesized and provided by Twist Bioscience. Heterologous nucleic acids encoding ScPHO13 and ScYKR070W were amplified form Saccharomyces cerevisiae genomic DNA. Heterologous nucleic acids encoding PKPHO13 and PKORF64, and PKORF423 were amplified from Pichia kudriavzevii genomic DNA. Heterologous nucleic acids encoding EcGPH was amplified form Escherichia coli genomic DNA. The transcriptional promoters cloned in front (5′) of each gene were constitutive and derived from P. kudriavzevii. The promoters for EcGPH, HsGPH, ScPHO13, PKPHO13, PKORF64, PKORF423, and ScYKR070W were the P. kudriavzevii TDHI promoter (pPKTDH1). The transcriptional terminators cloned behind (3′) of each gene were derived from S. cerevisiae. The terminators for EcGPH, HsGPH, ScPHO13, PKPHO13, PKORF64, PKORF423, and ScYKR070W were the Saccharomyces cerevisiae PYC2 terminator (tScPYC2). Additionally, a HIS3 marker was included in the heterologous expression cassette to complement the histidine auxotrophic deficiency in the background strain. This HIS3 marker comprised a transcriptional promoter, a HIS3 coding region, and a transcriptional terminator. The transcriptional promoter 5′ of HIS3 was the P. kudriavzevii TEFI promoter (pPKTEF1) and the transcriptional terminator 3′ of HIS3 was the S. cerevisiae TDH3 terminator (tScTDH3). All genetic elements were amplified from various templates with upstream and downstream homologous regions to neighboring genetic elements to drive correct assembly of the full-length pathway. The 5′ and 3′ ends of the expression cassette comprised regions homologous to the genomic sequences upstream and downstream of the P. kudriavzevii GPM1 locus, thereby facilitating integration of the heterologous nucleic acids encoding the glyceric acid pathway enzymes at the GPM1 locus in the P. kudriavzevii genome. Consequently, one or both copies of the PkGPM1 gene were deleted from the host genome; thus, genomic integration of the glyceric acid pathway simultaneously decreased or eliminated expression of PkGPM1.

All PCR products were purified and provided as exogenous nucleic acids to P. kudriavzevii. Transformation was carried out in a single step. Transformants were selected on CSM-His medium. Successful integration of all nucleic acids encoding glyceric acid pathway enzymes were confirmed by genetic sequencing of this locus and the flanking regions.

This example produced GPM minus strains which also comprised a glyceric acid pathway of the present disclosure with the following strain designations: LPK151910 (with EcGPH; SEQ ID NO: 1), LPK151909 (with HsGPH; SEQ ID NO: 3), LPK151954 (with ScPHO13; SEQ ID NO: 9), LPK152365 (with PkPHO13; SEQ ID NO: 10), LPK152367 (with PKORF64; SEQ ID NO: 11), LPK152366 (with PKORF423; SEQ ID NO:12), and LPK151912 (with ScYKR070W; SEQ ID NO: 13).

### Example 5

### Recombinant P. kudriavzevii Strains LPK151910, LPK151909, LPK151954, LPK152365, LPK152367, LPK152366, and LPK151912 Produced Increased Amounts of Glyceric Acid

Example 5 describes the culturing and analysis of recombinant host cells LPK151910, LPK151909, LPK151954, LPK152365, LPK152367, LPK152366, and LPK151912 from Example 4 (GPM minus, with genomic insertion of a glyceric acid pathway). All seven recombinant strains were cultured and analyzed by HPLC according to methods described above in Example 2.

In this example, all recombinant strains with a glyceric acid pathway produced a glyceric acid yield of 10-20% as compared to the lower, 6% product yield achieved when culturing the sister LPK151290 GPM minus strain that lacked a heterologous glyceric acid pathway. This example demonstrates, in accordance with the present disclosure, the expression of heterologous nucleic acids encoding a glyceric acid pathway in recombinant P. kudriavzevii increased glyceric acid yields as compared to a host cell lacking the heterologous glyceric acid pathway but otherwise genetically identical.

It should be noted that there are alternative ways of implementing the embodiments disclosed herein. Accordingly, the present embodiments are to be considered as illustrative and not restrictive; various modifications can be made without departing from the spirit of this disclosure. Furthermore, the claims are not to be limited to the details given herein, and are entitled their full scope and equivalents thereof.

What is claimed is:

1. A recombinant host cell, comprising:
a glyceric acid biosynthetic pathway, comprising heterologous nucleic acids encoding a 3-phosphoglycerate phosphatase and a 2-phosphoglycerate phosphatase;
wherein the expression of the heterologous nucleic acids produces glyceric acid.

2. The recombinant host cell of claim 1, wherein the recombinant host cell is a yeast cell.

3. The recombinant host cell of claim 2, wherein the yeast cell is of the Issatchenkia orientalis/Pichia fermentans clade.

4. The recombinant host cell of claim 3, wherein the yeast cell belongs to the genus Pichia, Issatchenkia, or Candida.

5. The recombinant host cell of claim 4, wherein the yeast cell is Pichia kudriavzevii.

6. The recombinant host cell of claim 2, wherein the yeast cell of the Saccharomyces clade.

7. The recombinant host cell of claim 6, wherein the yeast cell is Saccharomyces cerevisiae.

8. The recombinant host cell of claim 1, wherein the recombinant host cell is a prokaryotic cell.

9. The recombinant host cell of claim 8, wherein the prokaryotic cell belongs to the genus Escherichia, Corynebacterium, Bacillus, or Lactococcus.

10. The recombinant host cell of claim 9, wherein the prokaryotic cell is Escherichia coli, Corynebacterium glutamicum, Bacillus subtilis, or Lactococcus lactis.

11. The recombinant host cell of any one of claims 1-10, wherein the 3-phosphoglycerate phosphatase has an amino acid sequence selected from SEQ ID NO: 9 and an amino acid sequence having at least 90% identity with SEQ ID NO: 9.

12. The recombinant host cell of any one of claims 1-10, wherein the 3-phosphoglycerate phosphatase has an amino acid sequence selected from SEQ ID NO: 7 and an amino acid sequence having at least 90% amino acid identity with SEQ ID NO: 7.

13. The recombinant host cell of any one of claims 1-12, wherein the 2-phosphoglycerate phosphatase has an amino acid sequence selected from SEQ ID NO: 1 and an amino acid sequence having at least 90% amino acid identity with SEQ ID NO: 1.

14. The recombinant host cell of any one of claims 1-12, wherein the 2-phosphoglycerate phosphatase has an amino acid sequence selected from SEQ ID NO: 7 and an amino acid sequence having at least 90% amino acid identity with SEQ ID NO: 7.

15. The recombinant host cell of any one of claims 1-14, further comprising:
a heterologous nucleic acid encoding a mitochondrial external NADH dehydrogenase;
a nucleic acid encoding a water-forming NADH oxidase;
a heterologous nucleic acid encoding a glyceric acid transporter; or combinations of the foregoing.

16. The recombinant host cell of claim 15, wherein the mitochondrial external NADH dehydrogenase has an amino acid sequence selected from SEQ ID NO: 20 and an amino acid sequence having at least 90% identity with SEQ ID NO: 20.

17. The recombinant host cell of claim 15 or claim 16, wherein the water-forming NADH dehydrogenase has an amino acid sequence selected from SEQ ID NO: 21 and an amino acid sequence having at least 90% identity with SEQ ID NO: 21.

18. The recombinant host cell of any one of claim 15-17, wherein the glyceric acid transporter has an amino acid sequence selected from SEQ ID NO: 22, SEQ ID NO: 23, SEQ ID NO: 24, SEQ ID NO: 27, a protein having 90% sequence identity to SEQ ID NO: 22, a protein having 90% sequence identity to SEQ ID NO: 23, a protein having 90% sequence identity to SEQ ID NO: 24, and a protein having 90% sequence identity to SEQ ID NO: 27.

19. The recombinant host cell of any one of claims 1-18, further comprising:
a genetic disruption of an endogenous gene encoding a protein selected from phosphoglycerate mutase, phosphoglycerate dehydrogenase, enolase, glycerate 3-kinase, glycerate 2-kinase, glycerol-3-phosphate dehydrogenase, and combinations thereof.

20. The recombinant host cell of claim 19, wherein the phosphoglycerate mutase has an amino acid sequence selected from SEQ ID NO: 14 and an amino acid sequence having at least 90% identity with SEQ ID NO: 14.

21. The recombinant host cell of claim 19, wherein the phosphoglycerate mutase has an amino acid sequence selected from SEQ ID NO: 25 and an amino acid sequence having at least 90% identity with SEQ ID NO: 25.

22. The recombinant host cell of any one of claims 19-21, wherein the enolase has an amino acid sequence selected from SEQ ID NO: 26 and an amino acid sequence having at least 90% identity with SEQ ID NO: 26.

23. The recombinant host cell of any one of claims 19-21, wherein the enolase has an amino acid sequence selected from SEQ ID NO: 28 and an amino acid sequence having at least 90% identity with SEQ ID NO: 28.

24. The recombinant host cell of any one of claims 19-21, wherein the enolase has an amino acid sequence selected from SEQ ID NO: 29 and an amino acid sequence having at least 90% identity with SEQ ID NO: 29.

25. The recombinant host cell of any one of claims 19-24, wherein the glycerate 3-kinase has an amino acid sequence selected from SEQ ID NO: 16 and an amino acid sequence having at least 90% sequence identity with SEQ ID NO: 16.

26. The recombinant host cell of any one of claims 19-25, wherein the glycerate 2-kinase has an amino acid sequence selected from SEQ ID NO: 18 and an amino acid sequence having at least 90% sequence identity with SEQ ID NO: 18.

27. The recombinant host cell of any one of claims 19-26, wherein the recombinant host cell produces less than 5 g/l of ethanol, acetate, pyruvate, or combinations thereof.

28. The recombinant host cell of any one of claims 19-27, wherein the phosphoglycerate dehydrogenase has an amino acid sequence selected from SEQ ID NO: 15 and an amino acid sequence having at least 90% identity with SEQ ID NO: 15.

29. The recombinant host cell of any one of claims 19-27, wherein the phosphoglycerate dehydrogenase has an amino acid sequence selected from SEQ ID NO: 17 and an amino acid sequence having at least 90% identity with SEQ ID NO: 17.

30. The recombinant host cell of claim 28 or claim 29, wherein the recombinant host cell produces less than 5 g/l of serine.

31. The recombinant host cell of any one of claims 19-30, wherein the glycerol-3-phosphate dehydrogenase has an amino acid sequence selected from SEQ ID NO: 19 and an amino acid sequence having at least 90% sequence identity with SEQ ID NO: 19.

32. The recombinant host cell of claim 28 or claim 29, wherein the recombinant host cell produces less than 5 g/l of glycerol.

33. A method of producing glyceric acid, comprising:
culturing the recombinant host cell of claim 1 under conditions suitable to produce glyceric acid.

34. The method of claim 33, wherein the culturing is performed at an oxygen transfer rate greater than 10 mmol/l/hr.

35. The method of claim 33 or claim 34, wherein the culturing is performed at a temperature of about 25° C.-45° C.

36. The method of any one of claims 33-35, wherein the final pH of the fermentation broth is less than about pH 5.

37. The method of any one of claims 33-36, wherein the culturing produces at least 50 g/l glyceric acid.

38. The method of any one of claims 33-36, further comprising:
providing at least 100 g/l glucose to the recombinant host cell; and
producing at least 25% glyceric acid yield.

39. A method for producing glycerate esters, comprising:
recovering glyceric acid and glycerate salts from a cell-containing fermentation broth;
reacting the glyceric acid and glycerate salts with an alcohol in the presence of sulfuric acid to produce a glycerate ester; and
isolating the glycerate ester by distillation.

40. A method for producing acrylate esters, comprising:
reacting a glycerate ester with a reducing agent in the presence of a transition metal catalyst to produce an acrylate ester; and
isolating the acrylate ester;
wherein the transition metal catalyst is selected from vanadium compounds, molybdenum compounds, and rhenium compounds.

41. The method of claim 40, wherein the glycerate ester is selected from the group consisting of methyl glycerate, ethyl glycerate, butyl glycerate, and 2-ethylhexyl glycerate.

42. The method of claim 40 or claim 41, wherein the transition metal catalyst is methyltrioxorhenium or (NH₄)₆Mo₇O₂₄·4H₂O.

43. The method of any one of claims 40-42, wherein the reducing agent is selected from H₂, Na₂SO₃, benzene, toluene, 5-nonanol, 3-octanol, 2-octanol, 1-butanol, 3-pentanol, 2-methyl-1-butanol, isopropanol, and 2-ethylhexanol.

44. The method of any one of claims 40-43, wherein the acrylate ester is butyl acrylate or 2-ethylhexyl acrylate.

45. A poly(glycerate carbonate) compound comprising repeat units of formula (I):
—[OCH(C(═O)OR¹)CH₂OC(═O)]—
or a salt thereof; wherein
each R¹ is independently selected from hydrogen, optionally substituted C₁-C₅ alkyl, and optionally substituted aryl.

46. The compound of claim 45, which comprises about 25%. about 50%, about 75%, or about 90% non-petrochemical based components.
